SEQUENCE LISTING (65 sequences)

SEQ ID NO: 1 - protein, 707 aa, Homosapien
MLGSLVLRRKALAPRLLLRLLRSPTLRGHGGASGRNVTTGSLGEPQWLRVATGGRPGTSPALFSGRGAATGGRQGGRFDTKCLAAATWGRLPGPEETLPGQDSWNGVPSRAGLGMCALAAALVVHCYSKSPSNKDAALLEAARANNMQEVSRLLSEGADVNAKHRLGWTALMVAAINRNNSVVQVLLAAGADPNLGDDFSSVYKTAKEQGIHSLEDGGQDGASRHITNQWTSALEFRRWLGLPAGVLITREDDFNNRLNNRASFKGCTALHYAVLADDYRTVKELLDGGANPLQRNEMGHTPLDYAREGEVMKLLRTSEAKYQEKQRKREAEERRRFPLEQRLKEHIIGQESAIATVGAAIRRKENGWYDEEHPLVFLFLGSSGIGKTELAKQTAKYMHKDAKKGFIRLDMSEFQERHEVAKFIGSPPGYVGHEEGGQLTKKLKQCPNAVVLFDEVDKAHPDVLTIMLQLFDEGRLTDGKGKTIDCKDAIFIMTSNVASDEIAQHALQLRQEALEMSRNRIAENLGDVQISDKITISKNFKENVIRPILKAHFRRDEFLGRINEIVYFLPFCHSELIQLVNKELNFWAKRAKQRHNITLLWDREVADVLVDGYNVHYGARSIKHEVERRVVNQLAAAYEQDLLPGGCTLRITVEDSDKQLLKSPELPSPQAEKRLPKLRLEIIDKDSKTRRLDIRAPLHPEKVCNTI

SEQ ID NO: 2 - protein, 960 aa, Homosapien
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 3 - protein, 279 aa, Homospaien
MSLFDLFRGFFGFPGPRSHRDPFFGGMTRDEDDDEEEEEEGGSWGRGNPRFHSPQHPPEEFGFGFSFSPGGGIRFHDNFGFDDLVRDFNSIFSDMGAWTLPSHPPELPGPESETPGERLREGQTLRDSMLKYPDSHQPRIFGGVLESDARSESPQPAPDWGSQRPFHRFDDVWPMDPHPRTREDNDLDSQVSQEGLGPVLQPQPKSYFKSISVTKITKPDGIVEERRTVVDSEGRTETTVTRHEADSSPRGDPESPRPPALDDAFSILDLFLGRWFRSR

SEQ ID NO: 4 - DNA, 20 bp, Artificial
  Synthetic Sequence
gtttggccgtagcgatccgg

SEQ ID NO: 5 - DNA, 25 bp, Artificial
  Synthetic Sequence
caccggtttggccgtagcgatccgg

SEQ ID NO: 6 - DNA, 25 bp, Artificial
  Synthetic Sequence
aaacccggatcgctacggccaaacc

SEQ ID NO: 7 - DNA, 20 bp, Artificial
  Synthetic Sequence
gtacgatgaagaacaccctc

SEQ ID NO: 8 - DNA, 25 bp, Artificial
  Synthetic Sequence
caccggtacgatgaagaacaccctc

SEQ ID NO: 9 - DNA, 25 bp, Artificial
  Synthetic Sequence
aaacgagggtgttcttcatcgtacc

SEQ ID NO: 10 - DNA, 20 bp, Artificial
  Synthetic Sequence
agcgagtgtctcaagcgcat

SEQ ID NO: 11 - DNA, 25 bp, Artificial
  Synthetic Sequence
caccgagcgagtgtctcaagcgcat

SEQ ID NO: 12 - DNA, 25 bp, Artificial
  Synthetic Sequence
aaacatgcgcttgagacactcgctc

SEQ ID NO: 13 - DNA, 20 bp, Artificial
  Synthetic Sequence
agtagaaaagggcgacaacc

SEQ ID NO: 14 - DNA, 25 bp, Artificial
  Synthetic Sequence
caccgagtagaaaagggcgacaacc

SEQ ID NO: 15 - DNA, 25 bp, Artificial
  Synthetic Sequence
aaacggttgtcgcccttttctactc

SEQ ID NO: 16 - DNA, 20 bp, Artificial
  Synthetic Sequence
tttgctttccttctcagagc

SEQ ID NO: 17 - DNA, 25 bp, Artificial
  Synthetic Sequence
caccgtttgctttccttctcagagc

SEQ ID NO: 18 - DNA, 25 bp, Artificial
  Synthetic Sequence
aaacgctctgagaaggaaagcaaac

SEQ ID NO: 19 - DNA, 20 bp, Artificial
  Synthetic Sequence
acgctcaaccgagccccgcg

SEQ ID NO: 20 - DNA, 25 bp, Artificial
  Synthetic Sequence
caccgacgctcaaccgagccccgcg

SEQ ID NO: 21 - DNA, 25 bp, Artificial
  Synthetic Sequence
aaaccgcggggctcggttgagcgtc

SEQ ID NO: 22 - DNA, 20 bp, Artificial
  Synthetic Sequence
ccattgttcaatatcgtccg

SEQ ID NO: 23 - DNA, 25 bp, Artificial
  Synthetic Sequence
caccgccattgttcaatatcgtccg

SEQ ID NO: 24 - DNA, 25 bp, Artificial
  Synthetic Sequence
aaaccggacgatattgaacaatggc

SEQ ID NO: 25 - DNA, 20 bp, Artificial
  Synthetic Sequence
gagcgctgctcagatagcga

SEQ ID NO: 26 - DNA, 24 bp, Artificial
  Synthetic Sequence
caccgagcgctgctcagatagcga

SEQ ID NO: 27 - DNA, 24 bp, Artificial
  Synthetic Sequence
aaactcgctatctgagcagcgctc

SEQ ID NO: 28 - DNA, 20 bp, Artificial
  Synthetic Sequence
ctgcgtcttcacgtactcgg

SEQ ID NO: 29 - DNA, 25 bp, Artificial
  Synthetic Sequence
caccgctgcgtcttcacgtactcgg

SEQ ID NO: 30 - DNA, 25 bp, Artificial
  Synthetic Sequence
aaacccgagtacgtgaagacgcagc

SEQ ID NO: 31 - DNA, 20 bp, Artificial
  Synthetic Sequence
gaactcgaacattccaaacc

SEQ ID NO: 32 - DNA, 25 bp, Artificial
  Synthetic Sequence
caccggaactcgaacattccaaacc

SEQ ID NO: 33 - DNA, 25 bp, Artificial
  Synthetic Sequence
aaacggtttggaatgttcgagttcc

SEQ ID NO: 34 - DNA, 20 bp, Artificial
  Synthetic Sequence
gagagccacatatggcaaag

SEQ ID NO: 35 - DNA, 24 bp, Artificial
  Synthetic Sequence
caccgagagccacatatggcaaag

SEQ ID NO: 36 - DNA, 24 bp, Artificial
  Synthetic Sequence
aaacctttgccatatgtggctctc

SEQ ID NO: 37 - DNA, 20 bp, Artificial
  Synthetic Sequence
gggtcgggatgtctctggcg

SEQ ID NO: 38 - DNA, 24 bp, Artificial
  Synthetic Sequence
caccgggtcgggatgtctctggcg

SEQ ID NO: 39 - DNA, 24 bp, Artificial
  Synthetic Sequence
aaaccgccagagacatcccgaccc

SEQ ID NO: 40 - DNA, 20 bp, Artificial
  Synthetic Sequence
atccgggagatgatactgaa

SEQ ID NO: 41 - DNA, 25 bp, Artificial
  Synthetic Sequence
caccgatccgggagatgatactgaa

SEQ ID NO: 42 - DNA, 25 bp, Artificial
  Synthetic Sequence
aaacttcagtatcatctcccggatc

SEQ ID NO: 43 - DNA, 20 bp, Artificial
  Synthetic Sequence
gtaagccattacctgtgacc

SEQ ID NO: 44 - DNA, 24 bp, Artificial
  Synthetic Sequence
caccgtaagccattacctgtgacc

SEQ ID NO: 45 - DNA, 24 bp, Artificial
  Synthetic Sequence
aaacggtcacaggtaatggcttac

SEQ ID NO: 46 - DNA, 20 bp, Artificial
  Synthetic Sequence
aacggctccaccacgctcgg

SEQ ID NO: 47 - DNA, 25 bp, Artificial
  Synthetic Sequence
caccgaacggctccaccacgctcgg

SEQ ID NO: 48 - DNA, 25 bp, Artificial
  Synthetic Sequence
aaacccgagcgtggtggagccgttc

SEQ ID NO: 49 - DNA, 110 bp, Artificial
  Synthetic Sequence
catgcgagaaaagccttgtttgggctcacctgctaggttgcaggtttgggtcttcgagaagacctcaccggtttcatccaggatcgagcagttttagagctagaaatagc

SEQ ID NO: 50 - DNA, 20 bp, Artificial
  Synthetic Sequence
tgacctggaaaccatgccag

SEQ ID NO: 51 - DNA, 20 bp, Artificial
  Synthetic Sequence
aatgatctggagtggaggac

SEQ ID NO: 52 - DNA, 20 bp, Artificial
  Synthetic Sequence
cagaaccagcagaggtcaca

SEQ ID NO: 53 - DNA, 20 bp, Artificial
  Synthetic Sequence
agctgtgccactttcctttc

SEQ ID NO: 54 - DNA, 18 bp, Artificial
  Synthetic Sequence
gcgcaagaacgctcaacc

SEQ ID NO: 55 - DNA, 21 bp, Artificial
  Synthetic Sequence
tcaggttcctgagcagaagag

SEQ ID NO: 56 - DNA, 21 bp, Artificial
  Synthetic Sequence
gacctcaacgcacagtacgag

SEQ ID NO: 57 - DNA, 22 bp, Artificial
  Synthetic Sequence
aggagtcccatgatgagattgt

SEQ ID NO: 58 - DNA, 20 bp, Artificial
  Synthetic Sequence
agcgagcaacaggttggtga

SEQ ID NO: 59 - DNA, 21 bp, Artificial
  Synthetic Sequence
cacttccttctcgaagtgcca

SEQ ID NO: 60 - DNA, 20 bp, Artificial
  Synthetic Sequence
gatcattgctcctcctgagc

SEQ ID NO: 61 - DNA, 20 bp, Artificial
  Synthetic Sequence
acatctgctggaaggtggac

SEQ ID NO: 62 - DNA, 97 bp, Artificial
  Synthetic Sequence
tgctgttgacagtgagcgacaggtgcagatagttctgcaatagtgaagccacagatgtattgcagaactatctgcacctggtgcctactgcctcgga

SEQ ID NO: 63 - DNA, 97 bp, Artificial
  Synthetic Sequence
tgctgttgacagtgagcgaacggatcaatgagatcgtctatagtgaagccacagatgtatagacgatctcattgatccgtctgcctactgcctcgga

SEQ ID NO: 64 - DNA, 97 bp, Artificial; identical to SEQ ID NO: 63
  Synthetic Sequence

SEQ ID NO: 65 - DNA, 97 bp, Artificial
  Synthetic Sequence
tgctgttgacagtgagcgatccagataagtgacaagatcatagtgaagccacagatgtatgatcttgtcacttatctggactgcctactgcctcgga

(19) United States
(12) Patent Application Publication
(10) Pub. No.: US 2022/0323376 A1
(43) Pub. Date: Oct. 13, 2022

(54) METHODS AND COMPOSITIONS FOR SENSITIZING CANCER CELLS TO DRUG-INDUCED APOPTOSIS

(71) Applicant: New York University, New York, NY (US)

(72) Inventors: Iannis AIFANTIS, Brooklyn, NY (US); Xufeng CHEN, New York, NY (US); Christina GLYTSOU, New York, NY (US)

(21) Appl. No.: 17/608,103

(22) PCT Filed: May 1, 2020
(86) PCT No.: PCT/US2020/030975

§ 371 Date: Nov. 1, 2021

Related U.S. Application Data
(60) Provisional application No. 62/841,714, filed on May 1, 2019.

(51) Int. Cl. A61K 31/11; A61K 31/27; A61K 31/155; A61K 31/415; A61K 45/06; A61K 31/496; A61P 35/02; A61K 31/706

(52) U.S. Cl. CPC A61K 31/11; A61K 31/27; A61K 31/155; A61K 31/415; A61K 45/06; A61K 31/496; A61P 35/02; A61K 31/706

(57) ABSTRACT

Disclosed herein are methods of increasing the sensitivity of cancer cells to cell death by an apoptosis-inducing drug. Also disclosed herein are methods of treating cancer in a subject and combination therapeutics.

This application claims the priority benefit of U.S. Provisional Patent Application Ser. No. 62/841,714, filed May 1, 2019, which is hereby incorporated by reference in its entirety.

### FIELD

The present application is directed to methods of increasing the sensitivity of cancer cells to cell death by apoptosis-inducing drugs, methods of treating cancer in a subject, and combination therapeutics.

### BACKGROUND OF THE INVENTION

Acute Myeloid Leukemia (AML) is a hematopoietic neoplasm characterized by the proliferation and accumulation of aberrant immature myeloid progenitor cells. AML is associated with poor clinical outcome and high mortality, with an overall five-year survival rate of less than 15-30%. For AML patients, standard therapies often fail to achieve the complete remission, disease relapse is often fatal and salvage and bone marrow transplantation are only applicable to a few select AML patients, highlighting the need for novel targeted treatments. A number of such emerging treatments have targeted essential “hallmarks” of cancer, including the regulation of cancer cell survival by members of the BCL-2 protein family. Among these members, B-cell lymphoma 2 (BCL-2) is found upregulated in AML cells (Campos et al., “High Expression of bcl-2 Protein in Acute Myeloid Leukemia Cells is Associated with Poor Response to Chemotherapy,” Blood 81:3091-6 (1993)) and, specifically, in leukemic stem cells (LSC) (Lagadinou et al., “BCL-2 Inhibition Targets Oxidative Phosphorylation and Selectively Eradicates Quiescent Human Leukemia Stem Cells,” Cell Stem Cell 12:329-41 (2013)). BCL-2 overexpression is a poor-risk factor in AML and is associated with poor response to standard cytotoxic therapy (Campos et al., “High Expression of bcl-2 Protein in Acute Myeloid Leukemia Cells is Associated with Poor Response to Chemotherapy,” Blood 81:3091-6 (1993); and Tzifi et al., “The Role of BCL2 Family of Apoptosis Regulator Proteins in Acute and Chronic Leukemias,” Adv. Hematol. 2012:524308 (2012)).

Mechanistically, BCL-2 is the founding member of the BCL-2 protein family, and prevents programmed cell death by binding to proapoptotic members of the same family (Chao et al, “BCL-2 Family: Regulators of Cell Death,” Annu. Rev. Immunol. 16:395-419 (1998)). Following a death signal, the proapoptotic factors are released from BCL-2 and induce the oligomerization of BAX and BAK on the outer mitochondrial membrane (OMM) (Antignani et al., “How do Bax and Bak Lead to Permeabilization of the Outer Mitochondrial Membrane?,” Curr. Opin. Cell Biol. 18:685-9 (2006)). This is a crucial step for the release of cytochrome c from mitochondria to the cytosol where, together with dATP, APAF1 and caspase 9, it forms the apoptosome, a complex that subsequently activates the executioner caspases and thus apoptosis (Zou et al., “An APAF-1 Cytochrome c Multimeric Complex is a Functional Apoptosome that Activates Procaspase-9,” J. Biol. Chem. 274:11549-56 (1999); and Li et al., “Cytochrome c and dATP-Dependent Formation of Apaf-1/caspase-9 Complex Initiates an Apoptotic Protease Cascade,” Cell 91:479-89 (1997)).

In cancer cells, enhanced BCL-2 expression supports cell survival, as it leads to the suppression of mitochondrial-mediated apoptosis. Inhibiting BCL-2 via a selective BH3 mimetic such as Venetoclax has proven to be an efficient strategy to promote caspase-dependent cell death in AML (Pan et al., “Selective BCL-2 Inhibition by ABT-199 Causes on-Target Cell Death in Acute Myeloid Leukemia,” Cancer Discov. 4:362-75 (2014)). Venetoclax is an orally bioavailable drug that is approved for Chronic Lymphocytic Leukemia (CLL) and other hematological malignancies. Venetoclax recently received FDA approval for the treatment of newly-diagnosed elderly AML patients in combination with hypomethylating agents (DiNardo et al., “Safety and Preliminary Efficacy of Venetoclax with Decitabine or Azacitidine in Elderly Patients with Previously Untreated Acute Myeloid Leukaemia: a Non-Randomised, Open-Label, Phase 1b Study,” Lancet Oncol. 19:216-28 (2018)) (azacitidine or decitabine) as proposed (Bogenberger et al., “Ex Vivo Activity of BCL-2 Family Inhibitors ABT-199 and ABT-737 Combined with 5-azacytidine in Myeloid Malignancies,” Leuk. Lymphoma 56:226-9 (2015); and Bogenberger et al., “BCL-2 Family Proteins as 5-Azacytidine-Sensitizing Targets and Determinants of Response in Myeloid Malignancies,” Leukemia 28:1657-65 (2014)) or with low dose cytarabine (LDAC). However, approximately 30% of patients do not respond upfront and many AML patients still develop resistance while on treatment (Konopleva et al., “Efficacy and Biological Correlates of Response in a Phase II Study of Venetoclax Monotherapy in Patients with Acute Myelogenous Leukemia,” Cancer Discov. 6:1106-17 (2016)). This highlights the need for a greater mechanistic understanding of Venetoclax resistance, both in combination and as monotherapy. To that end, multiple combinational therapies for Venetoclax have been proposed, including CDK (CDK9) and MCL-1 inhibition as have been suggested (Ramsey et al., “A Novel MCL1 Inhibitor Combined with Venetoclax Rescues Venetoclax-Resistant Acute Myelogenous Leukemia,” Cancer Discov. 8:1566-81 (2018); Tahir et al., “Potential Mechanisms of Resistance to Venetoclax and Strategies to Circumvent it,” BMC Cancer 17:399 (2017); and Pan et al., “Synthetic Lethality of Combined Bcl-2 Inhibition and p53 Activation in AML: Mechanisms and Superior Antileukemic Efficacy,” Cancer Cell 32:748-60 e6 (2017)), with several of these combinations having entered clinical trials (Bogenberger et al., “Combined Venetoclax and Alvocidib in Acute Myeloid Leukemia,” Oncotarget. 8:107206-22 (2017)).

The present application is directed to overcoming these and other deficiencies in the art.

### SUMMARY OF THE INVENTION

A first aspect of the present application is directed to a method of increasing the sensitivity of cancer cells to cell death by an apoptosis-inducing drug, said method comprising administering to cancer cells an agent that modulates mitochondrial structure.

Another aspect of the present application is directed to a method of treating cancer in a subject, comprising selecting a subject having cancer; and administering to said subject an agent that modulates mitochondrial structure.

Another aspect of the present application is directed to a combination therapeutic comprising an agent that modulates mitochondrial structure and an apoptosis inducing drug.

Another aspect of the present application is directed to a method of treating cancer in a subject, comprising selecting a subject having cancer and administering to said subject a combination therapeutic comprising a BH3 protein mimetic and an agent that blocks autophagy.

Another aspect of the present application is a combination therapeutic comprising an agent that blocks autophagy and a BH3 protein mimetic.

As disclosed herein, The BCL-2 family plays important roles in cancers, including acute myeloid leukemia (AML). Venetoclax, a selective BCL-2 inhibitor, has received FDA approval for the treatment of AML and other cancers. However, drug resistance ensues after prolonged treatment, highlighting the need for a greater understanding of the underlying mechanisms. Using a genome-wide CRISPR/Cas9 screen in human AML cell line MOLM-13, genes whose inactivation sensitizes AML blasts to Venetoclax were identified. Genes involved in mitochondrial organization and function were significantly depleted throughout the screen, including the mitochondrial chaperonin CLPB, as well as OPA1, and HAX1. Notably, genes involved in mitochondrial biological processes also participate in the acquisition of Venetoclax resistance, highlighting the importance of mitochondrial structure and biological processes in BCL-2 inhibitor drug resistance. It was demonstrated that CLPB is upregulated in human AML, it is further induced upon acquisition of Venetoclax resistance and its ablation sensitizes AML to Venetoclax treatment. Furthermore, targeting CLPB synergizes with Venetoclax and Venetoclax/Azacitidine combination in a p53-independent manner. Mechanistically, CLPB maintains the mitochondrial cristae structure via its interaction with the cristae-shaping protein OPA1. Loss of CLPB leads to structural and functional defects of mitochondria, hence sensitizing cancer cells to apoptosis. Overall, these data indicate that targeting mitochondrial architecture provides an approach to circumvent BCL-2 inhibitor resistance.

### BRIEF DESCRIPTION OF THE DRAWINGS

FIGS. 1A-1G depict a genome-wide CRISPR screen that identifies genes controlling mitochondrial physiology as synthetic lethal with Venetoclax treatment in AML. FIG. 1A shows a schematic outline of the viability-based, genome wide CRISPR/Cas9 loss-of-function screen. FIG. 1B shows a volcano plot showing both positively and negatively selected genes in the CRISPR screen at day 8 post drug treatment. A number of positively and negatively selected genes and known regulators of Venetoclax resistance (both positively and negatively selected) are shown. FIGS. 1C-1D show frequency histograms of the delta CRISPR score of the negative control guides (top), and selected genes at day 8 (FIG. 1C) and day 16 (FIG. 1D) post drug treatment. FIG. 1E shows validation of selected genes in the CRISPR screen using a competition-based survival assay in MOLM-13. The normalized enrichment scores were calculated as shown in FIG. 2C. Data represent mean±SEM (n=4 for each sgRNA). FIG. 1F shows a Venn diagram of the negatively selected genes (“sensitizers”) in the CRISPR screen at day 8 (Log fold change <−1) and day 16 (Log fold change <−3) post drug treatment. FIG. 1G shows a STRING protein-protein interaction network of the 353 common negatively selected genes as defined in (FIG. 1F). The minimum required interaction score was set to 0.5, and the disconnected dots were removed. k-means clustering was applied with the number of clusters set to 6.

FIGS. 2A-2J depict a genome-wide loss-of-function CRISPR screen in AML combined with Venetoclax treatment. FIG. 2A shows a cell growth analysis of sgRNA library transduced MOLM-13 cells upon Venetoclax or DMSO treatment during the screen. FIG. 2B shows a volcano plot showing both positively and negatively selected genes in the CRISPR screen at day 16 post drug treatment. A number of positively and negatively selected genes and known regulators of Venetoclax resistance (both positively and negatively selected) are shown. FIG. 2C shows a schematic outline of the validation of the CRISPR/Cas9 loss-of-function screen. Formulas for calculating the normalized enrichment score are highlighted in the box. FIG. 2D shows the validation of the positively selected genes in the CRISPR screen in KASUMI-1 using a competition-based survival assay. The normalized enrichment scores were calculated as shown in (FIG. 2C). Data represent mean±SD (n=3 for each sgRNA). FIGS. 2E-2F shows IC₅₀ curves of Venetoclax in KASUMI-1 (FIG. 2E) and MOLM-13 (FIG. 2F) cell lines transduced with the indicated sgRNAs. Transduced AML cells were selected with puromycin and then treated with Venetoclax for 48 hours. Viable cells were measured by Cell-TiterGlo. Data represent mean±SD (n=3 for each group). FIG. 2G shows a Gene Ontology analysis of the positively selected genes in the screen (Log fold change >3). Of 21 input genes, 20 genes were mapped. FIG. 2H shows a venn diagram of the positively selected genes in the CRISPR screen at day 8 (Log fold change >0.5) and day 16 (Log fold change >2) post drug treatment. FIG. 2I shows a STRING protein-protein interaction network of the common positively selected genes defined in (FIG. 2H). The minimum required interaction score was set to 0.5, and the disconnected dots were removed. k-means clustering was applied with the number of clusters set to 6. FIG. 2J shows a Gene Ontology analysis of the negatively selected genes in the screen (Log fold change <−3). Of 1121 input genes, 1105 genes were mapped. Data with statistical significance are as indicated, *p<0.05, **p<0.01, ***p<0.001.

FIGS. 3A-3L depict the Mitochondrial response upon Venetoclax treatment and after acquisition of drug resistance. FIG. 3A shows representative electron micrographs of THP-1 treated with 4 μM Venetoclax or DMSO for 16 hrs. Scale bars represent 5 μm (upper panel) and 0.5 μm (lower panel). FIG. 3B shows the quantification of the maximal cristae width in 60 randomly selected mitochondria from 15 cells in experiments as in (A) (n=200 cristae per condition). Data represent mean±SEM of 3 independent experiments. FIG. 3C shows the quantification of the percentage of mitochondria with abnormal ultrastructure in experiments as in (FIG. 3A) (n=100 mitochondria per condition). Data represent mean±SEM of 3 independent experiments. FIG. 3D shows the quantification of the membrane potential loss after staining with TMRM in THP-1 cells treated with 4 μM Venetoclax or DMSO for 16 hrs. Data represent mean±SD (n=3). FIGS. 3E-3F show THP-1 treated with 4 μM Venetoclax for 16 hrs and equal amounts (30 μg) of cell lysates were separated by SDS-PAGE and immunoblotted using the indicated antibodies (FIG. 3E). L-OPA1, long forms of OPA1; S-OPA1, short forms of OPA1. The bar plot (FIG. 3F) shows the quantitative densitometric analysis of the ratio of long OPA1 forms to the total OPA1. Data represent mean±SEM of 3 independent experiments. FIG. 3G shows IC₅₀ curves of Venetoclax in parental or Venetoclax-resistant (VR) AML cell lines. Data represent mean±SD (n=3 for each cell line). FIG. 3H shows representative electron micrographs of mitochondria from parental (Par.) or Venetoclax-resistant (VR) MOLM-13. Scale bar represents 0.5 μm. FIG. 3I shows quantification of the maximal cristae width in 60 randomly selected mitochondria from 15 cells in experiment as in (H) (n=282 cristae per condition). Data represent mean±SEM. FIG. 3J shows western blot analysis in cell lysates from parental (Par.) or Venetoclax-resistant (VR) AML cell lines. FIG. 3K shows a Gene Ontology analysis of differentially expressed genes involved in mitochondrial processes in Venetoclax-resistant cells (MOLM-13 VR1) compared to the parental cell line (Log fold change >1 or <−1, FDR<0.05). FIG. 3L shows a scatter plot presenting the delta CRISPR score (FIG. 1B) plotted against the Log fold change from MOLM-13 VR RNA-seq (FIG. 5A). Shading corresponds to the common-log (p-value) which was generated using the geometric mean of the CRISPR screen p-value and the RNA-Seq FDR. Selected genes are highlighted. Data with statistical significance are as indicated, *p<0.05, ***p<0.001.

FIGS. 4A-4L depict the response of mitochondria upon acute Venetoclax treatment and after acquisition of drug resistance. FIG. 4A shows a scheme illustrating the mitochondrial cristae structure. Cristae lumen is highlighted. Arrow denotes the parameter of maximal Cristae Lumen Width (CLW, arrow) quantified in this study. FIG. 4B shows representative electron micrographs of MOLM-13 treated with 17 nM Venetoclax or DMSO for 16 hrs. Scale bars represent 0.5 μm. FIG. 4C shows quantification of the maximal cristae width (left panel; n=300 cristae per condition) and the percentage of mitochondria with abnormal ultrastructure (right panel) from 60 randomly selected mitochondria in experiments as in (FIG. 4B). Data represent mean±SEM of 4 independent experiments. FIG. 4D shows a representative histogram of the membrane potential analysis using TMRM in MOLM-13 cells treated with 35 nM Venetoclax or DMSO for 16 hrs. FIG. 4E shows THP-1 treated with 4 μM Venetoclax for 16 hrs and equal amounts (25 μg) of cell lysates were separated by SDS-PAGE and immunoblotted using the indicated antibodies. FIG. 4F shows cells treated with the indicated concentrations of Venetoclax or DMSO for 16 hrs and cell death was determined by flow cytometry using Annexin V. Data represent mean of Annexin V positive cell percentage±SD (n=3). FIG. 4G shows quantification of the number of cristae per mitochondrion in parental (Par.) or Venetoclax-resistant (VR) MOLM-13 cell lines (n=50 mitochondria per condition). Data represent mean±SD. FIG. 4H shows representative electron micrographs of mitochondria from parental (Par.) or Venetoclax-resistant (VR) MV4-11 cells. Scale bars represent 0.5 μm. FIG. 4I shows quantification of the maximal cristae width from 50 randomly selected mitochondria (left; n=200 cristae per condition) and the number of cristae per mitochondrion (right) in 50 mitochondria in experiments as in (FIG. 4H). Data represent mean±SEM. FIG. 4J shows quantification of the relative OPA1 band intensity from three independent experiments as in FIG. 3J. Data represent mean±SEM. FIG. 4K shows equal amounts (25 μg) of cell lysates separated by SDS-PAGE and immunoblotted using the indicated antibodies. FIG. 4L shows western blotting against BCL-2, MCL-1, BCL-XL and GRP75 in cell lysates (25 μg) of parental (Par.) or Venetoclax-resistant (VR) AML cell lines. Data with statistical significance are as indicated, *p<0.05, **p<0.01, ***p<0.001.

FIGS. 5A-5D depict transcriptional mitochondrial adaptation for acquisition of Venetoclax resistance. FIG. 5A shows a heatmap showing the differentially expressed genes in Venetoclax-resistant cells (MOLM-13 VR1) compared to the parental cell line (Log fold change >1 or <−1, FDR <0.05). Genes encoding mitochondrial proteins are highlighted on the left. FIG. 5B shows a heatmap showing the differentially expressed genes in Venetoclax-resistant cells (MV4-11 VR2) compared to the parental cell line (Log fold change >0.75 or <−0.75, FDR <0.05). Genes encoding mitochondrial proteins are highlighted on the left. FIG. 5C shows a venn diagram of the differentially expressed genes in MOLM-13 VR1 compared to MOLM-13 Par shown in (FIG. 5A) and in MV4-11 VR2 compared to MV4-11 Par shown in (FIG. 5B). FIG. 5D shows a Gene Ontology analysis of differentially expressed genes involved in mitochondrial processes in Venetoclax-resistant cells (MV4-11 VR1) compared to the parental cell line as shown in (FIG. 5B).

FIGS. 6A-6L show that targeting the mitochondrial protein CLPB synergizes with Venetoclax in AML. FIG. 6A shows a venn diagram of the 353 common negatively selected genes (“sensitizers”) throughout the screen, the 2221 core essential genes defined by Hart et al., “High-Resolution CRISPR Screens Reveal Fitness Genes and Genotype-Specific Cancer Liabilities,” Cell 163:1515-26 (2015), which is hereby incorporated by reference in its entirety, as well as 2220 genes which are upregulated in AML patients compared to healthy CD34⁺ HSPCs. Among these, 18 genes (listed in the table) were found non-essential and upregulated in AML patients. FIG. 6B shows a violin plot of CLPB mRNA expression level (FPKM) from RNA-sequencing in TCGA AML patients (200 patients) and normal human CD34⁺ HSPCs (6 healthy donors). FIG. 6C shows CLPB mRNA expression levels across diverse cancers from TCGA (log 2 FPKM). Sorted by median expression level. FIGS. 6D-6E show western blotting (FIG. 6D) and quantitative densitometric analysis (FIG. 6E) of CLPB protein levels of whole cell lysates from parental (Par.) and Venetoclax-resistant (VR) AML cell lines. FIGS. 6F-6G show IC₅₀ curves of Venetoclax in parental AML cells (FIG. 6F) and Venetoclax-resistant (VR) AML cells (FIG. 6G) transduced with CLPB sgRNAs or negative control (sgRosa). Transduced AML cells were selected with puromycin and then treated with Venetoclax for 48 hours. Viable cells were measured by Cell-TiterGlo. Data represent mean±SD (n=3 for each group). FIG. 6H shows bioluminescent images of mice transplanted with MOLM-13 cells transduced with sgRosa or sgCLPB #1. Mice were administrated with vehicle or Venetoclax (Ven) from day 6 to day 20 post transplantation as described in FIG. 9E. The same mice are depicted at each time-point (n=4 mice per group). FIG. 6I shows quantification of bioluminescence emitted from the whole body of each mouse described in (FIG. 6H) at the indicated time points. FIG. 6J shows flow cytometry analysis of GFP⁺ sgRNA-expressing leukemia cells in peripheral blood of MOLM-13 leukemia recipient mice described in (FIG. 6H) at the indicated time points. FIG. 6K shows Kaplan-Meier survival curves of the MOLM-13 leukemia recipient mice described in (FIG. 6H). The p-values were determined using Log rank Mantel-Cox test. FIG. 6L shows western blotting in whole cell lysates from sorted GFP⁺ sgRosa-expressing leukemia cells in the bone marrow of MOLM-13 leukemia recipient mice treated with vehicle or Venetoclax (Ven), as described in (FIG. 6H). Animals were sacrificed when they showed signs of late stage leukemia. Data with statistical significance are as indicated, *p<0.05, **p<0.01, ***p<0.001, N.S., not significant.

FIGS. 7A-7K show that CLPB is highly expressed in AML cell lines and its protein levels correlate to the cellular sensitivity to Venetoclax. FIG. 7A shows CLPB mRNA expression levels across diverse cancer cell lines from Cancer Cell Line Encyclopedia (CCLE, Broad Institute). FIG. 7B shows CLPB dependency across diverse cancer cell lines from the Achilles shRNA knockdown project. Data were obtained from CCLE (Broad Institute). FIG. 7C shows CLPB mRNA expression levels in Venetoclax-resistant MOLM-13 (MOLM-13 VR) relative to the parental clones (MOLM-13 Par). Expression data were generated by RNA-sequencing as shown in FIG. 5A and were normalized to parental clones (n=2). FIG. 71D shows MOLM-13 treated with Venetoclax (IC₅₀) for 10 days and the alive cells were isolated using Histopaque. Lysates from the alive cells were separated by SDS-PAGE and immunoblotted using the indicated antibodies. FIG. 7E shows quantification of the relative CLPB band intensity from three independent experiments as in (FIG. 7D). Data represent mean±SEM. FIG. 7F shows IC₅₀ curves of Venetoclax in multiple AML cell lines. Cells were treated with Venetoclax for 48 hrs. Viable cells were measured by Cell-TiterGlo. Data represent mean±SD (n=3). FIG. 7G shows equal amounts (30 μg) of protein from AML cell lines separated by SDS-PAGE and immunoblotted using the indicated antibodies. FIG. 7H shows quantitative densitometric analysis of CLPB levels from experiment (FIG. 7G). FIGS. 7I-7J show IC₅₀ curves of Cytarabine (left), Idarubicin (middle) and JQ-1 (right) in MOLM-13 (FIG. 7I) and MV4-11 (FIG. 7) cell lines transduced with sgCLPBs (#1 and #2) or sgRosa. Transduced AML cells were selected with puromycin and then treated with the indicated drugs for 48 hrs. Viable cells were measured by Cell-TiterGlo. Data represent mean±SD (n=3 for each group). FIG. 7K shows MOLM-13 treated with Idarubicin (10 nM) for 2 days and the alive cells were isolated using Histopaque. Lysates from the alive cells were separated by SDS-PAGE and immunoblotted using the indicated antibodies. Data with statistical significance are as indicated, *p<0.05, **p<0.01.

FIGS. 8A-8E depict the dose dependent effect of CLPB in sensitizing AML cells to BCL-2 inhibition. FIG. 8A shows western blotting against CLPB and ACTIN in cell lysates of MOLM-13 infected with sgRNAs targeting CLPB or negative control (sgRosa). Rep: replica. FIG. 8B shows relative expression level of CLPB mRNA in MOLM-13 transduced with CLPB-targeting shRNAs or negative control (shRenilla). Expression data were generated by real time PCR and were normalized to the shRenilla control (n=3). FIG. 8C shows competition-based viability assays of MOLM-13 transduced with CLPB-targeting shRNAs or negative control (shRenilla). Plotted are GFP⁺ percentages measured during 14 days in culture and normalized to Day 4. GFP⁺ percentages were then normalized to shRenilla control at each time point (n=6). FIG. 8D shows the validation of the synergistic effect of CLPB knockdown and Venetoclax treatment using a competition-based survival assay in MOLM-13. The normalized enrichment scores were calculated as shown in FIG. 2C (n=6). FIG. 8E shows competition-based viability assays in non-AML cell lines (melanoma: SK-MEL-239, B-ALL: Nalm6, T-ALL: CuTLL1, CML: K562) transduced with sgRNAs targeting CLPB or negative control (sgRosa). Plotted are GFP⁺ percentages measured during 20 days in culture and normalized to Day 4 (n=6). Data with statistical significance are as indicated, ***p<0.001, N.S.: not significant.

FIGS. 9A-9J show that targeting CLPB overcomes Venetoclax resistance in AML. FIG. 9A shows IC₅₀ curves of Venetoclax in THP-1 cells transduced with CLPB sgRNAs or negative control (sgRosa). Transduced AML cells were selected with puromycin and then treated with Venetoclax for 48 hours. Viable cells were measured by Cell-TiterGlo. Data represent mean±SD (n=3 for each group). FIG. 9B shows validation of the synergistic effect of CLPB depletion with Venetoclax treatment using a competition-based survival assay in MOLM-13 (left), MV4-11 (middle) and THP-1 (right) AML cell lines. Plotted are GFP⁺ cell percentages measured during 6 days in culture and normalized to Day 0 of drug treatment. Negative control (sgRosa) and two independent sgRNAs targeting CLPB are shown in the graphs. Data represent mean±SD (n=12 for MOLM-13, n=9 for MV4-11, n=12 for THP-1). FIG. 9C shows the synergistic effect of CLPB depletion and BCL-2 inhibition in Venetoclax-resistant (VR) MOLM-13 and MV4-11 cells (two clones for each cell line). Competition-based survival assay was carried out as described in FIG. 2C. Data represent mean±SD (n=6 for MOLM-13-VR1, n=3 for MOLM-13-VR2, MV4-11-VR1 and MV4-11-VR2). FIG. 9D shows IC₅₀ curves of Venetoclax in parental and Venetoclax-resistant (VR) MOLM-13 (left) and MV4-11 (right) AML cells transduced with CLPB sgRNAs or negative control (sgRosa) as described in FIG. 6G. Data represent mean±SD (n=3 for each group). FIGS. 9E-9F show schematic outlines of the mouse models for in vivo validating the synergistic effects of CLPB ablation and Venetoclax treatment using parental (FIG. 9E) and Venetoclax-resistant (VR; FIG. 9F) MOLM-13 xenografts. FIG. 9G shows bioluminescent images of mice transplanted with Venetoclax-resistant (VR) MOLM-13 cells transduced with sgRosa or sgCLPB #1. Mice were administrated with vehicle or Venetoclax from day 6 to day 20 post transplantation as described in (FIG. 9F). The same mice are depicted at each time-point (n=4 mice per group). FIG. 9H shows quantification of bioluminescence emitted from the whole body of each mouse described in (FIG. 9G) at the indicated time points. FIG. 9I shows flow cytometry analysis of GFP⁺ sgRNA-expressing leukemia cells in peripheral blood of MOLM-13 VR leukemia recipient mice described in (FIG. 9G) at the indicated time points. FIG. 9J shows Kaplan-Meier survival curves of the MOLM-13 VR leukemia recipient mice described in (FIG. 9G). The p-values were determined using Log rank Mantel-Cox test. Data with statistical significance are as indicated, *p<0.05, **p<0.01, ***p<0.001.

FIGS. 10A-10K depict the subcellular localization of CLPB and its function in regulating apoptosis. FIG. 10A shows endogenous CLPB co-localizes with the mitochondrial marker TOM20. Representative confocal images of THP-1 and HeLa cells stained for TOM20 and CLPB. Scale bars represent 10 μm. FIG. 10B shows a Gene Ontology analysis of genes co-expressed with CLPB in TCGA AML patients (Pearson's Correlation >0.4). Of 222 input genes, 221 genes were mapped. FIG. 10C shows representative dot plots showing the expression of two genes (OPA3 and ATAD3A) involved in mitochondrial organization control, positively correlated to CLPB expression in AML. FIG. 10D shows representative electron micrographs (left) and quantification of the maximal cristae width (right; n=300 cristae per condition) of MOLM-13 cells transduced with CLPB sgRNAs or control (sgRosa) and treated with 17 nM Venetoclax or DMSO for 16 hrs. Scale bars represent 500 nm. Data represent mean±SEM of two independent experiments. FIG. 10E shows MOLM-13 and MV4-11 infected with the indicated constructs and treated with 35 nM and 70 nM Venetoclax, respectively, for 16 hrs. Membrane potential was measured by staining with TMRM and analyzed using flow cytometry. Data represent mean±SD (n=3). FIGS. 10F-10G depict BH3 profiling. Kinetics plots of mitochondrial membrane potential measured by JC-1 dye upon addition of BIM (FIG. 10F) and BID (FIG. 10G) peptides in THP-1 transduced cells with CLPB sgRNAs or control (sgRosa). Alamethicin and CCCP served as positive controls, while PUMA 2A as a negative control. Data was normalized to 1% DMSO controls. FIG. 10H shows MOLM-13 and THP-1 infected with the indicated constructs and treated with 35 nM and 4 μM Venetoclax, respectively, for 16 hrs. Cell lysates (30 μg) were separated by SDS-PAGE and immunoblotted using the indicated antibodies. FIG. 10I shows silver staining of endogenous CLPB immunoprecipitation (IP) in THP-1 cells. Asterisk denotes the predicted size of CLPB. FIG. 10J shows whole THP-1 lysates immunoprecipitated with anti-CLPB coupled to magnetic Protein-G beads. Bound proteins were separated by SDS-PAGE and immunoblotted against CLPB and HAX1. Input was diluted 1:10. FIG. 10K shows liver mitochondrial lysates immunoprecipitated with anti-CLPB coupled to magnetic Protein-G beads. Co-precipitated proteins were separated by SDS-PAGE and immunoblotted against CLPB and HAX1. Input was diluted 1:10. Data with statistical significance are as indicated, **p<0.01, ***p<0.001.

FIGS. 11A-11J show that CLPB loss induces mitochondrial ultrastructure defects sensitizing AML cells to mitochondria-mediated cell death FIG. 11A shows representative electron micrographs of THP-1 transduced with sgRNAs targeting CLPB or control (sgRosa) and treated with 4 μM Venetoclax or DMSO for 16 hrs. Scale bars represent 2 μm (upper panel) and 500 nm (lower panel). FIG. 11B shows quantification of the percentage of mitochondria with abnormal ultrastructure in experiments as in (A) (n=100 mitochondria per condition; upper panel). Quantification of the maximal cristae width in 60 randomly selected mitochondria from 15 cells in experiments as in (A) (n=200 cristae per condition; lower panel). FIG. 11C shows equal amounts (30 μg) of protein from THP-1 infected with sgRNAs targeting CLPB or control (sgRosa) were separated by SDS-PAGE and immunoblotted using the indicated antibodies. FIG. 11D shows quantitative densitometric analysis of the ratio of OPA1 long (L-OPA1) versus short (S-OPA1) forms in experiments as in (C). Data represent mean±SEM of 8 independent experiments. FIG. 11E shows representative electron micrographs and morphometric analysis of BAX/BAK double-knockout MOLM-13 cells transduced with sgRNAs targeting CLPB or control (sgRosa). Scale bars represent 500 nm. Morphometric analysis was performed in 60 randomly selected mitochondria (n=200 cristae per condition) in two independent experiments. FIG. 11F shows quantification of the membrane potential loss after staining with TMRM in THP-1 infected with sgRNAs targeting CLPB or control (sgRosa) and treated with 4 μM Venetoclax or DMSO for 16 hours. Data represent mean±SD (n=3 for each group). FIG. 11G shows BH3 profiling: Mitochondrial depolarization measured by JC-1 in permeabilized THP-1 transduced with sgRosa or sgCLPB upon stimulation with BIM and BID peptides. Depolarization (%) was calculated based on the area under the curve for each condition and normalized to CCCP positive control and 1% DMSO as negative control as previously described (Ryan et al., “BH3 Profiling in Whole Cells by Fluorimeter or FACS,” Methods 61:156-64 (2013), which is hereby incorporated by reference in its entirety). FIG. 11H shows isolated mitochondria from THP-1 transduced as indicated treated with recombinant cBID for 30 min and centrifuged at 12.000×g for 10 min. Pellet and supernatant (SN) of each sample were separated with SDS-PAGE and immunoblotted for cytochrome c (cyt c). % Cyt c release is calculated as the percentage of the supernatant to the total (pellet and supernatant) cytochrome c band intensity. FIG. 11I shows AML cells transduced with sgRNAs targeting CLPB or control (Rosa), treated with Venetoclax or DMSO for 16 hrs and cell death was determined by flow cytometry using Annexin V. Data represent mean±SD (n=3). FIG. 11J shows liver mitochondrial lysates immunoprecipitated with anti-CLPB coupled to magnetic Protein-G beads. Co-precipitated proteins were separated by SDS-PAGE and immunoblotted against CLPB and OPA1. Input was diluted 1:10. Data with statistical significance are as indicated, *p<0.05, **p<0.01, ***p<0.001, N.S., not significant.

FIGS. 12A-12H show that CLPB ablation leads to cell growth suppression in AML in vitro and in vivo. FIG. 12A shows oxygen consumption rate (OCR), respiration (bar plot) and extracellular acidification rate (ECAR) of wild-type and CLPB-knockout MOLM-13 determined by Seahorse Extracellular Flux Analysis. Data represent mean±SEM (n=5). FIG. 12B shows representative flow cytometry plots showing EdU cell cycle analysis of wild-type and CLPB knockout MV4-11 (left panel). Bar charts depict the mean percentage of cell populations±SD (n=3) in MV4-11 or THP-1 AML cells (right panel). FIG. 12C shows cell growth analysis of AML cells transduced with sgRNAs targeting CLPB or control (sgRosa) (mean±SD, n=3). FIG. 12D shows a schematic outline of the mouse model of CLPB dependency in AML. FIG. 12E shows bioluminescent images of mice transplanted with MOLM-13 cells transduced with sgRosa (n=3) or sgCLPB #1 (n=6). Representative images of two mice per sgRNA construct are shown. The same mice are depicted at each time-point. FIG. 12F shows quantification of bioluminescence emitted from the whole body of each mouse transduced with sgRosa or sgCLPB #1 construct at the indicated time points. FIG. 12G shows flow cytometry analysis of GFP⁺ sgRNA-expressing leukemia cells in peripheral blood of MOLM-13 leukemia recipient mice at indicated time points. FIG. 12H shows Kaplan-Meier survival curves of recipient mice transduced with sgRosa and sgCLPB #1 are plotted. The p-values were determined using Log rank Mantel-Cox test. Data with statistical significance are as indicated, *p<0.05, **p<0.01, ***p<0.001, N.S., not significant.

FIGS. 13A-13H show that CLPB deficiency amplifies proapoptotic signals by inducing mitochondrial stress response. FIG. 13A shows a heatmap showing the differentially expressed genes in MOLM-13 transduced with sgRNAs targeting CLPB or control (sgRosa) at day 6 and day 8 post transduction (Log fold change >1 or <−1, False Discovery Rate <0.05 in all samples). Common genes are shown in the heatmap. FIG. 13B shows a KEGG pathway enrichment analysis of the differentially expressed genes in MOLM-13 transduced with sgRNAs targeting CLPB or control (sgRosa) at day 6 post transduction. FIG. 13C shows an Ingenuity Pathway Analysis of the differentially expressed genes (Log fold change >1 or <−1, False Discovery Rate <0.05 in all samples) in MOLM-13 transduced with two independent sgRNAs targeting CLPB (left panel, sgCLPB #1; right panel, sgCLPB #2) or control (sgRosa) at day 6 post transduction revealing activation of the ATF4 upstream pathway. FIG. 13D shows a qPCR analysis of ATF4 transcripts in MOLM-13 transduced with sgRNAs targeting CLPB or control (sgRosa) (mean±SEM, n=3). FIG. 13E shows enrichment score plots from Gene-set enrichment analysis (GSEA) using the mitochondrial stress expression signature defined by Quiros et al., “Multi-Omics Analysis Identifies ATF4 as a Key Regulator of the Mitochondrial Stress Response in Mammals,” J. Cell Biol. 216:2027-45 (2017), which is hereby incorporated by reference in its entirety, and the RNA-Seq data of MOLM-13 transduced with sgRNAs targeting CLPB or control (sgRosa) at day 6 post transduction. FDR, false discovery rate; NES, normalized enrichment score. FIG. 13F shows a qPCR analysis of the relative expression levels of NOXA (PMAIP1) mRNA, PUMA (BBC3) mRNA and HRK mRNA in MOLM-13 transduced with CLPB-targeting sgRNAs or control (sgRosa) at day 6 post transduction (mean±SD, n=3). FIG. 13G shows a heatmap showing the significantly differentially detected metabolites in MOLM-13 transduced with two independent sgRNAs targeting CLPB or control (sgRosa) at day 6 or day 8 post transduction (p<0.05). Average intensity of each metabolite was shown. FIG. 13H shows a metabolome pathway enrichment analysis of top altered metabolites in (G). Data with statistical significance are as indicated, *p<0.05, **p<0.01.

FIGS. 14A-14H show that CLPB deficiency leads to mitochondrial oxidative stress. FIG. 14A shows enrichment score plots from Gene-set enrichment analysis (GSEA) using the mitochondrial stress expression signature defined by Quirós et al and the RNA-Seq data of MOLM-13 transduced with CLPB-sgRNAs at day 8 post transduction. FDR, false discovery rate; NES, normalized enrichment score. FIG. 14B shows MOLM-13 infected with sgRNAs targeting CLPB or control (Rosa) stained with MitoSOX and analyzed by flow cytometry. Graphs show MitoSOX-mean fluorescence intensity (MFI) upon antimycin A treatment relative to the basal MFI. FIG. 14C shows hierarchical clustering of 16 metabolites of FIG. 13G and 62 differentially expressed genes in CLPB sgRNAs transduced MOLM-13 of FIG. 13A. Each row represents an individual metabolite and each column represents an individual gene. Genes were ordered by centered correlation and complete linkage according to their correlation coefficient. Shading key indicates positive or negative correlation values, respectively. The scale represents the Spearman correlation values from 1 to −1. FIG. 14D shows qPCR analysis of ATF4 transcripts in MV4-11 transduced with sgRNAs targeting CLPB or control (sgRosa) (mean±SEM, n=3). FIGS. 14E-14F show qPCR analysis of CHOP transcripts in MOLM-13 (FIG. 14E) and MV4-11 (FIG. 14F) transduced with sgRNAs targeting CLPB or control (sgRosa) (mean±SEM, n=3). FIG. 14G shows qPCR analysis of NOXA (left) and PUMA (right) transcripts in MV4-11 transduced with sgRNAs targeting CLPB or control (sgRosa) (mean±SEM, n=3). FIG. 14H shows BH3 profiling: Mitochondrial depolarization measured by JC-1 in permeabilized THP-1 transduced with sgRosa or sgCLPB upon stimulation with PUMA, BMF-γ and MS1 peptides. % Depolarization was calculated based on the area under the curve for each condition and normalized to CCCP positive control and 1% DMSO as negative control. Data with statistical significance are as indicated, *p<0.05, **p<0.01, ***p<0.001.

FIGS. 15A-15E show that p53-deficient MOLM-13 exhibit increased Venetoclax IC₅₀. FIG. 15A shows western blotting against p53 and ACTIN in lysates from MOLM-13 cells infected with sgRNAs targeting TP53 or negative control (Rosa). Three different clones of p53-knockout (KO) were blotted. sgRNAs targeting TP53 (B10 and B11) were used for further experiments. FIG. 15B shows IC₅₀ curves of Venetoclax in KASUMI-1 cells transduced with CLPB sgRNAs or sgRosa. Transduced AML cells were selected with puromycin and then treated with Venetoclax for 48 hours. Viable cells were measured by Cell-TiterGlo. Data represent mean±SD (n=3 for each group). FIG. 15C shows validation of the synergistic effect of CLPB depletion and BCL-2 inhibition in KASUMI-1 cells using competition-based survival assay as described in FIG. 2C. Data represent mean±SD (n=3). FIG. 15D shows western blotting against NOXA and GAPDH in cell lysates (30 μg) of p53-wild type and p53-knockout (sgTP53) MOLM-13 cells transduced with sgCLPB #1 or sgRosa. FIG. 15E shows qPCR analysis of ATF4, NOXA, PUMA and HRK transcripts in p53-wild type and p53-knockout (sgTP53) MOLM-13 cell lines transduced with sgCLPB #1 or sgRosa (mean±SEM, n=3). Data with statistical significance are as indicated, *p<0.05, **p<0.01, ***p<0.001, N.S.: not significant.

FIGS. 16A-16D demonstrate that CLPB targeting overcomes p53-mediated Venetoclax resistance and sensitizes AML cells to combined Venetoclax and Azacitidine treatment. FIG. 16A shows the synergistic effect of CLPB depletion and BCL-2 inhibition in TP53-knockout (KO) MOLM-13 cell lines (two clones, B10 and B11) generated as shown in FIGS. 15A-15E. Plotted are GFP⁺ percentages measured during 4 days in culture and normalized to Day 0 of drug treatment. Negative control (sgRosa) and two independent sgRNAs targeting CLPB are shown in the graphs. Data represent mean±SD (n=4). FIG. 16B shows IC₅₀ curves of Venetoclax in p53 wild-type (WT) and p53-deficient (KO) (two clones, B10 and B11) MOLM-13 cells transduced with sgCLPB #1 or sgRosa. Transduced AML cells were selected with puromycin and then treated with Venetoclax for 48 hours. Viable cells were measured by Cell-TiterGlo. Data represent mean±SD (n=6 for each group). FIG. 16C. shows validation of the synergistic effect of CLPB depletion and Venetoclax+Azacitidine combined treatment using a competition-based survival assay in MOLM-13 (left) and MV4-11 (right) cells. Plotted are GFP⁺ percentages measured during 6 days (for MOLM-13) or 4 days (for MV4-11) in culture and normalized to Day 0 of drug treatment. Negative control (sgRosa) and two independent sgRNAs targeting CLPB are shown in the graphs. Data represent mean±SD (n=6 for MOLM-13 and n=3 for MV4-11). FIG. 16D shows a schematic outline of this study. Targeting the mitochondrial CLPB sensitizes AML cells to Venetoclax treatment by 1) promoting apoptotic cristae remodeling and 2) inducing mitochondrial stress response which will amplify the programmed cell death pathway. Data with statistical significance are as indicated, ***p<0.001.

FIGS. 17A-17G show that targeting the mitochondrial chaperonin, CLPB, sensitizes human AML to BH3 mimetics. FIG. 17A shows the molecular structures of the CLPB inhibitors, compound 3 (left) and compound 6 (right). FIG. 17B shows the IC₅₀ curves of compound 6 (C6, left) and compound 3 (C3, right), in human AML cell lines, MOLM-13, MV4-11, THP-1 and OCI-AML3, and human non-AML cell line, HEK-293T. IC₅₀ values of each condition are shown in the box below the curves. FIG. 17C shows the normalized survival of human AML cell lines, MOLM-13 (left), MV4-11 (middle) and THP-1 (right), under the treatment of Venetoclax with the indicated amount of compound 3. FIG. 17D shows the ICs curves of Venetoclax in human AML cell lines, MOLM-13 (left), MV4-11 (middle) and THP-1 (right), in combination with the indicated amount of compound 3. FIG. 17E shows the IC₅₀ curves of Venetoclax (left) and Venetoclax plus compound 3 (used at the IC₅₀ of each cell line, right) in human AML cell lines, MOLM-13, MV4-11, THP-1 and OCI-AML3. IC₅₀ values of each condition are shown in the box of the right of the curves. FIG. 17F shows the IC₅₀ curves of Venetoclax (left) and compound 3 (right) in parental AML cell lines, MOLM-13-P and MV4-11-P, and Venetoclax resistant AML cell lines, MOLM-13-VR and MV4-11-VR. IC50 values of each condition are shown in the box of the right of the curves. FIG. 17G shows MOLM-13 (left) and KASUMI-1 (right) cells transduced with sgRNAs targeting CLPB (sgCLPB #1 and sgCLPB #2) or Rosa control (sgRosa) were treated with the indicated concentrations of AMG176 or DMSO for 22 hrs and cell death was determined by flow cytometry using Annexin V. Data represent mean of Annexin V positive cell percentage±SD.

FIGS. 18A-18J show that BH3-mimetics resistance in AML is coupled to OPA1 overexpression, while OPA1 targeting re-sensitizes resistant cells to the drugs. FIG. 18A shows the representative electron micrographs of mitochondria from parental (Par.) or Venetoclax-resistant (VR) MOLM-13 cells. FIGS. 18B-18C show the morphometric analyses in experiment as in A (n=282 cristae per condition; mean±SEM). FIG. 18D shows the western-blot analysis in cell lysates from Par. or VR AML cell lines. FIG. 18E shows the western-blot analysis in cell lysates from Par. or Venetoclax+Azacitidine-resistant (VAR) MOLM-13 cell lines. FIG. 18F shows the western-blot analysis in cell lysates from Par. or Venetoclax+Azacitidine-resistant (VAR) Kasumi-1 cell lines. FIG. 18G shows the western blotting in lysates from sorted GFP+ MOLM-13 cells in the bone marrow of leukemia recipient mice treated with vehicle or Venetoclax (Ven). Animals were sacrificed when they showed signs of late stage leukemia. FIG. 18H shows the Cell death analysis of THP-1 cells transduced with the indicated sgRNAs and treated with 4 μM venetoclax or DMSO for 16 hrs. FIG. 18I shows the competition-based survival assay in MOLM-13 VAR cells. Data represent mean±SD (n=3 for each sgRNA). FIG. 18J shows the IC₅₀ curves of Venetoclax+Azacitidine in parental or VAR MOLM-13 cell lines transduced with sgRNAs targeting OPA1. Transduced cells were selected with puromycin and then treated with Venetoclax for 48 hrs. Viable cells were measured by Cell-TiterGlo. Data represent mean±SD (n=3 for each group).

FIGS. 19A-19L show that MFN2-mediated mitophagy is a potential mechanism of cellular resistance to BH3 mimetics in human AML. FIG. 19A shows MFN2 dependency across diverse cancer cell lines from the Cancer Dependency Map Project. The higher the scaled Bayesian factor is, the more essential the gene is. Data were obtained from depmap portal (Broad Institute). FIG. 19B shows MFN2 protein normalized spectral in human AML cell lines compared to non-AML hematologic malignancies. Data were obtained from the Nusinow et al., “Quantitative Proteomics of Cancer Cell Line Encyclopedia,” Cell 180(2):387-402.e16 (2020), which is hereby incorporated by reference in its entirety. FIG. 19C shows the western-blot analysis in cell lysates from healthy human cord blood CD34+ cells and human AML cell lines. FIG. 19D shows the quantitative densitometric analysis of MFN2 levels from experiment C. FIG. 19E show the correlation score of MFN2 mRNA expression vs AML patients' cell response to drug (IC₅₀). Data were obtained from BeatAML. Shading shows correlation. FIG. 19F shows the IC₅₀ curves of AMG176 in parental or AMG176-resistant (MR) human AML cell lines. MOLM-13 (left), KASUMI-1 (right). FIG. 19G shows the western-blot analysis in cell lysates from Parental (Par.) or AMG176-resistant (MR). FIG. 19H shows the quantitative densitometric analysis of MFN2 levels relative to ACTIN from experiment G. FIGS. 19I-19J show the western-blot analysis in lysates from AML cells treated with 100 μM Chloroquine and 1 μM/1 μM Antimycin A (AA)+Oligomycin (Oligo) for 16 hrs. Bar graph depicts the quantification of the ratio of LC3B-II/LC3B-I band intensity. FIG. 19K shows the mean mtDNA copy number measured by qPCR, relative to healthy human cord blood CD34+ cells. Data represent mean±SEM (n=3 for each group). FIG. 19L shows the median fluorescence intensity of MitoTracker Green. Data represent mean±SD (n=3 for each group). Data with statistical significance are as indicated, *p<0.05, **p<0.01.

FIGS. 20A-20L show that inhibition of autophagy synergizes with BH3 mimetic drugs in human AML. FIG. 20A shows the IC₅₀ curves of Venetoclax+Azacitidine (V+A) in mouse AML cell line, RN2, in combination with the indicated amount of autophagy inhibitor Chloroquine (CQ). FIG. 20B shows the IC₅₀ curves of Chloroquine (CQ) in mouse AML cell line, RN2, in combination with the indicated amount of Venetoclax+Azacitidine (V+A). FIG. 20C shows the synergistic effect of Venetoclax+Azacitidine (V+A) in combination with Chloroquine (CQ) in mouse AML cell line, RN2. ZIP model was used to calculate the delta-score of synergisms of the combination treatments. FIGS. 20D-20E show the synergistic effects of Venetoclax+Azacitidine (V+A) in combination with autophagy inhibitor, DC661 (D) and Chloroquine (E) in human AML cell lines, MOLM-13, MV4-11 and KASUMI-1. ZIP model was used to calculate the delta-score of synergisms of the combination treatments. FIGS. 20F-20G show the synergistic effects of Venetoclax+Azacitidine (V+A) in combination with DC661 in Venetoclax+Azacitidine-resistant AML cell lines, MOLM-13-VAR2 (F) and MV4-11-VAR1 (G). ZIP model was used to calculate the delta-score of synergisms of the combination treatments. FIG. 20H shows the IC₅₀ curves of AMG176 in human AMG176-resistant MOLM-13 cells (MR; two clones) in combination with DMSO or Chloroquine (40 μM). FIG. 20I shows the IC₅₀ curves of AMG176 in human AMG176-resistant KASUMI-1 cells (MR; two clones) in combination with DMSO or Chloroquine (40 μM). FIG. 20J shows parental MOLM-13 and AMG176-resistant MOLM-13 cells (two clones) were treated with DMSO or 40 μM Chloroquine (CQ) or 300 nM AMG176 or the combination for 16 hrs and cell death was determined by flow cytometry using Annexin V. Data represent mean of Annexin V positive cell percentage±SEM. FIG. 20K shows parental MOLM-13 and AMG176-resistant MOLM-13 cells (two clones) were treated with DMSO or 40 μM Chloroquine (CQ) or 6 nM Venetoclax or the combination for 16 hrs and cell death was determined by flow cytometry using Annexin V. Data represent mean of Annexin V positive cell percentage f SD. FIG. 20L shows parental MOLM-13 and AMG176-resistant MOLM-13 cells (two clones) were treated with DMSO or 40 μM Chloroquine (CQ) or 3 nM Venetoclax plus 30 nM AMG176 or the combination for 16 hrs and cell death was determined by flow cytometry using Annexin V. Data represent mean of Annexin V positive cell percentage±SD.

FIGS. 21A-21B show that blocking mitophagy synergistically enhances the killing efficacy of BH3 mimetic drugs in human AML. FIG. 21A shows the western blotting against DRP1 when phosphorylated at Ser616 [pDRP1 (Ser616)], the mitochondrial marker TOM20 and the cytosolic TUBULIN in lysates from Parental (Par.) and AMG176-resistant (MR) human AML cell lines. FIG. 21B shows the IC₅₀ curves of AMG176 in human AML cell lines (MOLM-13 on the left; KASUMI-1 on the right) in combination with the indicated amount of the DRP1 and mitophagy inhibitor Mitochondrial division inhibitor-1 (Mdivi1).

### DETAILED DESCRIPTION OF THE INVENTION

The present application is directed to the treatment of cancers and/or cancer cells. In particular, the present application describes the treatment of cancers and/or cancer cells resistant to apoptosis inducing drugs.

Accordingly, a first aspect of the present application is directed to a method of increasing the sensitivity of cancer cells to cell death by an apoptosis-inducing drug, said method comprising administering to cancer cells an agent that modulates mitochondrial structure.

In embodiments of the various aspects of the present application, the cancer is leukemia and the cancer cells are leukemic cells. In some embodiments, the leukemic cells are chronic lymphocytic leukemia (CLL) cells, T-cell prolymphocytic leukemia (T-PLL) cells, acute myeloid leukemia (AML) cells, myelodysplastic syndromes (MDS) cells, chronic myelomonocytic leukemia (CMML) cells, early T cell progenitor leukemia (ETP-ALL) cells, or combinations thereof. In some embodiments, the leukemic cells are leukemic stem cells. In some embodiments, the leukemic cells are p53 deficient leukemic cells. In some embodiments, the leukemic cells are mutant TP53 cells. In some embodiments, the leukemic cells are p53 proficient leukemic cells.

In embodiments of the various aspects of the present application, the cancer is lymphoma and the cancer cells are lymphoma cells. In embodiments, the lymphoma cells are follicular lymphoma cells, diffuse large B cell lymphoma cells, marginal zone lymphoma cells, non-hodgkin lymphoma cells, primary CNS lymphoma cells, mantle cell lymphoma cells, high grade B-cell lymphoma cells, burkitt lymphoma cells, richter syndrome cells, T cell lymphoma cells, cutaneous T-cell lymphoma cells, small lymphocytic lymphoma cells, or combinations thereof.

In embodiments of the various aspects of the present application, the cancer is myeloma and the cancer cells are myeloma cells. In some embodiments, the myeloma cells are multiple myeloma cells, recurrent plasma cell myeloma cells, myelodysplastic syndrome cells, or combinations thereof.

In embodiments of the various aspects of the present application, the cancer is a tumor and the cancer cells are tumor cells. In some embodiments, the tumor cells are blastic plasmacytoid dendritic cell neoplasm cells, metastatic prostate carcinoma cells, dendritic cell neoplasm cells, breast neoplasm cells, breast cancer cells, neuroblastoma cells, waldenstrom macroglobulinemia cells, non-small cell lung cancer cells, ovarian tumor cells, or combinations thereof.

In embodiments of the various aspects of the present application, the cancer cells are mammalian cells. In some embodiments, the mammalian cells are human cells.

In embodiments of the various aspects of the present application, the cancer cells are resistant to drug-induced apoptosis, and administering the agent that modulates mitochondrial structure reverses said resistance.

In embodiments of the various aspects of the present application, the apoptosis-inducing drug is a BH3 protein mimetic. BH3 protein mimetics are known in the art and described, for example, in Merino et al., “BH3-Mimetic Drugs: Blazing the Trail for New Cancer Medicines,” Cancer Cell 34(6): 879-91 (2018); and Chonghaile, “BH3 Mimetics: Weapons of Cancer Cell Destruction,” Science Translational Medicine 11(475):eaaw5311 (2019), which are each hereby incorporated by reference in their entirety. Suitable BH3 protein mimetics include those that inhibit BCL-2, BCL-XL, BCL-W, MCL-1, or combinations thereof.

In some embodiments, the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

In some embodiments, the BH3 protein mimetic is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax)

In embodiments of the various aspects of the present application, the agent that modulates mitochondrial structure is an agent that inhibits the expression and/or activity of caseinolytic peptidase B protein homolog (CLPB). The nucleotide sequence encoding human CLPB is known in the art and readily accessible via the National Center for Biotechnology Information database (NCBI) (Gene Id 81570). The amino acid sequence of human (Homo sapiens) CLPB is also known in the art and readily accessible via the UniProtKB database (Q9H078.1). That amino acid sequence (SEQ ID NO:1) is as follows:

MLGSLVLRRKALAPRLLLRLLRSPTLRGHGGASGRNVTTGSLGEPQWLRVA
TGGRPGTSPALFSGRGAATGGRQGGRFDTKCLAAATWGRLPGPEETLPGQD
SWNGVPSRAGLGMCALAAALVVHCYSKSPSNKDAALLEAARANNMQEVSRL
LSEGADVNAKHRLGWTALMVAAINRNNSVVQVLLAAGADPNLGDDFSSVYK
TAKEQGIHSLEDGGQDGASRHITNQWTSALEFRRWLCLPACVLITREDDFN
NRLNNRASFKGCTALHYAVLADDYRTVKELLDGGANPLQRNEMGHTPLDYA
REGEVMKLLRTSEAKYQEKQRKREAEERRREPLEQRLKEHIIGQESAIATV
GAAIRRKENGWYDEEHPLVFLFLGSSGIGKTELAKQTAKYMHKDAKKGFIR
LDMSEFQERHEVAKFIGSPPGYVGHEEGGQLTKKLKQCPNAVVLFDEVDKA
HPDVLTIMLQLFDEGRLTDGKGKTIDCKDAIFIMTSNVASDEIAQHALQLR
QEALEMSRNRIAENLGDVQISDKITISKNEKENVIRPILKAHFRRDEFLGR
INEIVYFLPFCHSELIQLVNKELNFWAKRAKQRHNITLLWDREVADVLVDG
YNVHYGARSIKHEVERRVVNQLAAAYEQDLLPGGCTLRITVEDSDKQLLKS
PELPSPQAEKRLPKLRLEIIDKDSKTRRLDIRAPLHPEKVCNTI.

In some embodiments, the sequence of CLPB according to these or any other embodiments described herein comprise one or more (e.g., 1, 2, 3, 4, 5 or more) amino acid insertions, deletions, modifications (e.g. substitution of one amino acid for another) compared to SEQ ID NO:1, or are otherwise substantially identical (e.g. having a sequence of at least 80%, 81%, 82%, 83%, 84%, 85%, 86%, 87%, 88%, 89%, 90%, 91%, 92%, 93%, 94%, 95%, 96%, 97%, 98%, 99% or more identical) with the entire sequence of SEQ ID NO:1. It is contemplated that such variations retain the mitochondrial structure related function of, for example, SEQ ID NO:1. For example, polypeptides or proteins comprising an amino acid sequence having one or more (e.g. 1, 2, 3, 4, 5, or more) conservative amino acid substitutions relative to SEQ ID NO:1, but retaining the function of SEQ ID NO:1 are encompassed. Nucleic acid molecules encoding such variants are also contemplated. Thus, isoforms of the human CLPB of SEQ ID NO:1 are also contemplated, as are both orthologs and homologs of the human CLPB of SEQ ID NO:1. For example, the Canis lupus familiaris (dog) ortholog (NCBI Gene Id 476815) and the Felis catus (domestic cat) ortholog (NCBI Gene Id 101083619).

Suitable CLPB inhibitors include, without limitation protein or peptide inhibitors, e.g., anti-CLPB antibodies, nucleic acid molecule inhibitors, e.g, a CLPB antisense oligonucleotide inhibitor, and small molecule inhibitors of CLPB.

Suitable CLPB inhibitors for use in the methods include those known in the art, see, e.g., Martin et al., “Screening and Evaluation of Small Organic Molecules as ClpB Inhibitors and Potential Antimicrobials,” J. Med. Chem 56: 7177-7189 (2013), which is hereby incorporated by reference in its entirety. In some embodiments, the agent that inhibits the expression and/or activity of CLPB is a compound of Formula I:

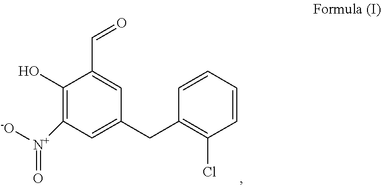

or a derivative thereof.

In some embodiments, the agent that inhibits the expression and/or activity of CLPB is a compound of Formula II:

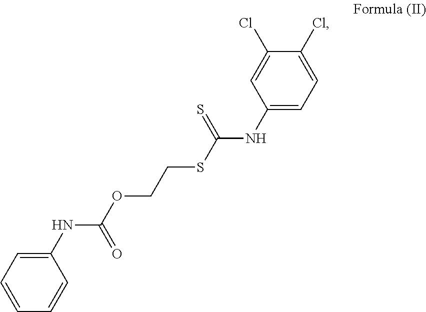 [SMILES: O=C(Nc1ccccc1)OCCSC(=S)Nc1ccc(Cl)c(Cl)c1]

or a derivative thereof.

In some embodiments, the agent that inhibits the expression and/or activity of CLPB is guanidinium hydrochloride (CH₆ClN₃), or a derivative thereof.

In embodiments of the various aspects of the present application, the agent that modulates mitochondrial structure is an agent that inhibits the expression and/or activity of Optic atrophy protein 1 (OPA1). The nucleotide sequence encoding human OPA1 is known in the art and readily accessible via the National Center for Biotechnology Information database (Gene Id 4976). The amino acid sequence of human (Homo sapiens) OPA1 is also known in the art and readily accessible via the UniProtKB database (O60313 (OPA1_HUMAN)). That amino acid sequence (SEQ ID NO:2) is as follows:

MWRLRRAAVACEVCQSLVKHSSGIKGSLPLQKLHLVSRSIYHSHHPTLKLQ
RPQLRTSFQQFSSLTNLPLRKLKFSPIKYGYQPRRNFWPARLATRLLKLRY
LILGSAVGGGYTAKKTFDQWKDMIPDLSEYKWIVPDIVWEIDEYIDFEKIR
KALPSSEDLVKLAPDFDKIVESLSLLKDFFTSGSPEETAFRATDRGSESDK
HFRKVSDKEKIDQLQEELLHTQLKYQRILERLEKENKELRKLVLQKDDKGI
HHRKLKKSLIDMYSEVLDVLSDYDASYNTQDHLPRVVVVGDQSAGKTSVLE
MIAQARIFPRGSGEMMTRSPVKVTLSEGPHHVALFKDSSREFDLTKEEDLA
ALRHEIELRMRKNVKEGCTVSPETISLNVKGPGLQRMVLVDLPGVINTVTS
GMAPDTKETIFSISKAYMQNPNAIILCIQDGSVDAERSIVTDLVSQMDPHG
RRTIFVLTKVDLAEKNVASPSRIQQIIEGKLFPMKALGYFAVVTGKGNSSE
SIEAIREYEEEFFQNSKLLKTSMLKAHQVTTRNLSLAVSDCFWKMVRESVE
QQADSFKATRFNLETEWKNNYPRLRELDRNELFEKAKNEILDEVISLSQVT
PKHWEEILQQSLWERVSTHVIENIYLPAAQTMNSGTFNTTVDIKLKQWTDK
QLPNKAVEVAWETLQEEFSRFMTEPKGKEHDDIFDKLKEAVKEESIKRHKW
NDFAEDSLRVIQHNALEDRSISDKQQWDAAIYFMEEALQARLKDTENAIEN
MVGPDWKKRWLYWKNRTQEQCVHNETKNELEKMLKCNEEHPAYLASDEITT
VRKNLESRGVEVDPSLIKDTWHQVYRRHFLKTALNHCNLCRRGFYYYQRHF
VDSELECNDVVLFWRIQRMLAITANTLRQQLINTEVRRLEKNVKEVLEDFA
EDGEKKIKLLTGKRVQLAEDLKKVREIQEKLDAFIEALHQEK

In some embodiments, the sequence of OPA1 according to these or any other embodiments described herein comprise one or more (e.g., 1, 2, 3, 4, 5 or more) amino acid insertions, deletions, modifications (e.g. substitution of one amino acid for another) compared to SEQ ID NO:2, or are otherwise substantially identical (e.g. having a sequence of at least 80%, 81%, 82%, 83%, 84%, 85%, 86%, 87%, 88%, 89%, 90%, 91%, 92%, 93%, 94%, 95%, 96%, 97%, 98%, 99% or more identical) with the entire sequence of SEQ ID NO:2. It is contemplated that such variations retain the mitochondrial structure related function of, for example, SEQ ID NO:2. For example, polypeptides or proteins comprising an amino acid sequence having one or more (e.g. 1, 2, 3, 4, 5, or more) conservative amino acid substitutions relative to SEQ ID NO:2, but retaining the function of SEQ ID NO:2 are encompassed. Nucleic acid molecules encoding such variants are also contemplated. Thus, isoforms of the human OPA1 of SEQ ID NO:2 are also contemplated, as are both orthologs and homologs of the human OPA1 of SEQ ID NO:2. For example, the Canis lupus familiaris (dog) ortholog (NCBI Gene Id 477129) and the Felis catus (domestic cat) ortholog (NCBI Gene Id 101086671).

Suitable OPA1 inhibitors include, without limitation protein or peptide inhibitors, e.g., anti-OPA1 antibodies, nucleic acid molecule inhibitors, e.g, an OPA1 antisense oligonucleotide inhibitor, and small molecule inhibitors of OPA1.

In some embodiments, the agent that inhibits the expression and/or activity of OPA1 is N-(1,5-dimethyl-3-oxo-2-phenyl-2,3-dihydro-1H-pyrazol-4-YL)-3-methyl-1-PH+ (MYLS22) (see Herkenne et al., “Developmental and Tumor Angiogenesis Requires the Mitochondria-Shaping Protein Opal,” Cell Metabolism (Apr. 20, 2020), which is hereby incorporated by reference in its entirety), or derivative thereof.

In embodiments of the various aspects of the present application, the agent that modulates mitochondrial structure is an agent that inhibits the expression and/or activity of HCLS1-associated protein X-1 (HAX1). The nucleotide sequence encoding human HAX is known in the art and readily accessible via the National Center for Biotechnology Information database (Gene Id 10456). The amino acid sequence of human (Homo sapiens) HAX1 is also known in the art and readily accessible via the UniProtKB database (O00165 (HAX1_HUMAN)). That amino acid sequence (SEQ ID NO:3) is as follows:

MSLFDLFRGFFGFPGPRSHRDPFFGGMTRDEDDDEEEEEEGGSWGRGNPRF
HSPQHPPEEFGFGFSFSPGGGIRFHDNFGFDDLVRDFNSIFSDMGAWTLPS
HPPELPGPESETPGERLREGQTLRDSMLKYPDSHQPRIFGGVLESDARSES
PQPAPDWGSQRPFHRFDDVWPMDPHPRTREDNDLDSQVSQEGLGPVLQPQP
KSYFKSISVTKITKPDGIVEERRTVVDSEGRTETTVTRHEADSSPRGDPES
PRPPALDDAFSILDLFLGRWFRSR.

In some embodiments, the sequence of HAX1 according to these or any other embodiments described herein comprise one or more (e.g., 1, 2, 3, 4, 5 or more) amino acid insertions, deletions, modifications (e.g. substitution of one amino acid for another) compared to SEQ ID NO:3, or are otherwise substantially identical (e.g. having a sequence of at least 80%, 81%, 82%, 83%, 84%, 85%, 86%, 87%, 88%, 89%, 90%, 91%, 92%, 93%, 94%, 95%, 96%, 97%, 98%, 99% or more identical) with the entire sequence of SEQ ID NO:3. It is contemplated that such variations retain the mitochondrial structure related function of, for example, SEQ ID NO:3. For example, polypeptides or proteins comprising an amino acid sequence having one or more (e.g. 1, 2, 3, 4, 5, or more) conservative amino acid substitutions relative to SEQ ID NO:3, but retaining the function of SEQ ID NO:3 are encompassed. Nucleic acid molecules encoding such variants are also contemplated. Thus, isoforms of the human HAX1 of SEQ ID NO:3 are also contemplated, as are both orthologs and homologs of the human HAX1 of SEQ ID NO:3. For example, the Canis lupus familiaris (dog) ortholog (NCBI Gene Id 480134) and the Felis catus (domestic cat) ortholog (NCBI Gene Id 101097787).

Suitable HAX inhibitors include, without limitation protein or peptide inhibitors, e.g., anti-HAX1 antibodies, nucleic acid molecule inhibitors, e.g, a HAX1 antisense oligonucleotide inhibitor, and small molecule inhibitors of HAX1.

Another aspect of the present application is directed to a method of treating cancer in a subject, comprising selecting a subject having cancer; and administering to said subject an agent that modulates mitochondrial structure.

Selecting a subject may involve selecting a subject having cancer, selecting a subject having cancer that is resistant to treatment with apoptosis inducing drug, and/or selecting a subject at risk of having a form of cancer that is resistant to treatment with apoptosis inducing drug, e.g., resistant to treatment with a BH3 mimetic. Suitable cancers to be treated in accordance with this aspect of the disclosure are disclosed supra.

In some embodiments, selecting comprises selecting a patient having or at risk of having leukemia, e.g., acute myeloid leukemia, T cell acute lymphocytic leukemia (T-ALL), early T cell progenitor leukemia (ETP-ALL), or chronic lymphocytic leukemia, that is resistant to treatment with an apoptosis inducing drug. In some embodiments, the leukemia is resistant to treatment with a BH3 protein mimetic.

Suitable subjects in accordance with methods described herein include, without limitation, a mammal, e.g., a human. In certain embodiments, the selected subject is female, e.g., a premenopausal female or a postmenopausal female. In some embodiments, the subject is male. Additional suitable subjects include, but are not limited to, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In accordance with this aspect of the disclosure, an agent that modulates mitochondrial structure is administered to the subject having cancer. Suitable agents that modulate mitochondrial structure are described supra. In some embodiments, the agent that modulates mitochondrial structure is administered to the subject as part of a combination therapy or therapeutic. In some embodiments, the combination therapeutic comprises the agent that modulates mitochondrial structure and an apoptosis inducing drug.

As used herein, the term “combination therapy” or “combination therapeutic” refers to the administration of two or more therapeutic agents, e.g., an agent that modulates mitochondrial structure, an apoptosis inducing drug, a chemotherapeutic drug, and combinations thereof. In some embodiments, the combination therapy is co-administered in a substantially simultaneous manner, such as in a single capsule or other delivery vehicle having a fixed ratio of active ingredients. In some embodiment, the combination therapy is administered in multiple capsules or delivery vehicles, each containing an active ingredient. In some embodiments, the therapeutic agents of the combination therapy are administered in a sequential manner, either at approximately the same time or at different times. In all of the embodiments, the combination therapy provides beneficial effects of the drug combination in treating cancer, particularly in treatment-resistant cancers as described herein.

In some embodiments, the agents of the combination therapeutic, i.e., the agent that modulates mitochondria structure and the apoptosis inducing drug are administered concurrently. In other embodiments, the agent that modulates mitochondrial structure is administered prior to administering the apoptosis inducing drug.

In some embodiments, the agent that modulates mitochondrial structure in the combination therapeutic is an agent that inhibits expression and/or activity of CLPB, OPA1, HAX1, or combinations thereof. Suitable agents for inhibiting these proteins include those described supra.

In some embodiments, the apoptosis inducing drug of the combination therapeutic is a BH3 protein mimetic that inhibits BCL-2, BCL-XL, BCL-W, MCL-1, or combinations thereof. Suitable BH3 protein mimetics include, e.g., navitoclax, venetoclax, S55746, BLC201, S63845, AMG-176, AZD-5991, ABT-737, AT101, A-1331852, A-1155463, TW-37, A-1210477, and Sabutoclax, as described supra.

In some embodiments, the combination therapeutic further comprises a chemotherapeutic drug.

In embodiments of the various aspects of the present application, the chemotherapeutic drug is a hypomethylating agent. Suitable hypomethylating agents include, without limitation, 4-amino-1-[(2R,3R,4S,5R)-3,4-dihydroxy-5-(hydroxymethyl)oxolan-2-yl]-1,3,5-triazin-2-one (azacitidine), 4-amino-1-[(2R,4S,5R)-4-hydroxy-5-(hydroxymethyl)oxolan-2-yl]-1,3,5-triazin-2-one (decitabine), 4-amino-1-[(2R,3S,4S,5R)-3,4-dihydroxy-5-(hydroxymethyl)oxolan-2-yl]pyrimidin-2-one (cytarabine), (8S,10S)-10-{[(2R,4S,5S,6S)-4-amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione (doxorubicin), (8S,10S)-8-acetyl-10-([(2R,4S,5S,6S)-4-amino-5-hydroxy-6-methyloxan-2-yl]oxy)-6,8,11-trihydroxy-1-methoxy-5,7,8,9,10,12-hexahydrotetracene-5,12-dione (daunorubicin), (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (epirubicin), (7S,9S)-9-acetyl-7-{[(2R,4S,5S,6S)-4-amino-5-hydroxy-6-methyloxan-2-yl]oxy}-6,9,11-trihydroxy-5,7,8,9,10,12-hexahydrotetracene-5,12-dione (idarubicin), anthracene-1,2-dione (anthracenedione), (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Adriamycin), 1,4-dihydroxy-5,8-bis((2-[(2-hydroxyethyl)amino]ethyl)amino)-9,10-dihydroanthracene-9,10-dione (mitoxantrone), and derivatives and/or combinations thereof.

In some embodiments, the hypomethylating agent is 4-amino-1-[(2R,3R,4S,5R)-3,4-dihydroxy-5-(hydroxymethyl)oxolan-2-yl]-1,3,5-triazin-2-one (azacitidine).

In some embodiments, the combination therapeutic further comprises a CDK9 inhibitor and/or a MCL-1 inhibitor.

In embodiments of the various aspects of the present application, suitable CDK9 inhibitors include, without limitation 2-(2-chlorophenyl)-5,7-dihydroxy-8-[(3S,4R)-3-hydroxy-1-methylpiperidin-4-yl]chromen-4-on (Alvocidib), (16E)-14-methyl-20-oxa-5,7,14,27-tetrazatetracyclo[19.3.1.12,6.18,12]heptacosa-1(25),2(27),3,5,8,10,12(26),16,21,23-decaene (Zotiraciclib), 4-[(2,6-dichlorobenzoyl)amino]-N-piperidin-4-yl-1H-pyrazole-5-carboxamide (AT-7519), (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide (AZD-4573), TP-1287, 2-[2-chloro-4-(trifluoromethyl)phenyl]-5,7-dihydroxy-8-[(2R,3S)-2-(hydroxymethyl)-1-methylpyrrolidin-3-yl]chromen-4-one (Voruciclib), 5-fluoro-4-(4-fluoro-2-methoxyphenyl)-N-[4-[(methylsulfonimidoyl)methyl]pyridin-2-yl]pyridin-2-amine (BAY-1251152), N-[5-[(5-tert-butyl-1,3-oxazol-2-yl)methylsulfanyl]-1,3-thiazol-2-yl]piperidine-4-carboxamide (SNS-032), (2R)-2-[[6-(benzylamino)-9-propan-2-ylpurin-2-yl]amino]butan-1-ol (Roscovitine), 2-[(2S)-1-[3-ethyl-7-[(1-oxidopyridin-1-ium-3-yl)methylamino]pyrazolo[1,5-a]pyrimidin-5-yl]piperidin-2-yl]ethanol (dinaciclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (Abemaciclib), 4-[(2,6-dichlorobenzoyl)amino]-N-piperidin-4-yl-1H-pyrazole-5-carboxamide (AT7519) and derivatives and/or combinations thereof.

In embodiments of the various aspects of the present application, suitable MCL-1 inhibitors include, without limitation, 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), N-[4-(2-tert-butylphenyl)sulfonylphenyl]-2,3,4-trihydroxy-5-[(2-propan-2-ylphenyl)methyl]benzamide (TW-37), (Z)-4-[(1S,2S,8R,17S,19R)-12-hydroxy-8,21,21-trimethyl-5-(3-methylbut-2-enyl)-8-(4-methylpent-3-enyl)-14,18-dioxo-3,7,20-trioxahexacyclo[15.4.1.0^{2,15}.0^{2,19}.0^{4,13}.0^{6,11}]docosa-4(13),5,9,11,15-pentaen-19-yl]-2-methylbut-2-enoic acid (Gambogic acid), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), [4,5-dichloro-1-[4,5-dichloro-2-(2-hydroxybenzoyl)-1H-pyrrol-3-yl]pyrrol-2-yl]-(2-hydroxyphenyl)methanone (maritoclax), 2-[4-[(4-bromophenyl)sulfonylamino]-1-hydroxynaphthalen-2-yl]sulfanylacetic acid (UMI-77), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), (2R)-2-[5-[3-chloro-2-methyl-4-[2-(4-methylpiperazin-1-yl)ethoxy]phenyl]-6-(4-fluorophenyl)thieno[2,3-d]pyrimidin-4-yl]oxy-3-[2-[[2-(2-methoxyphenyl)pyrimidin-4-yl]methoxy]phenyl]propanoic acid (MIK665/S64315), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD5991), and derivatives and/or combinations thereof.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and a compound of Formula I:

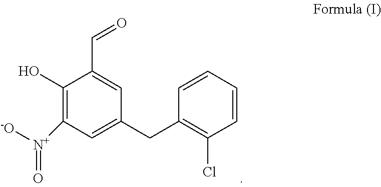

In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

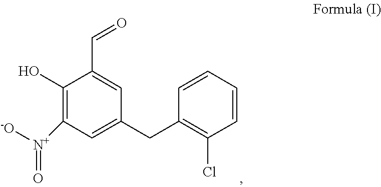

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

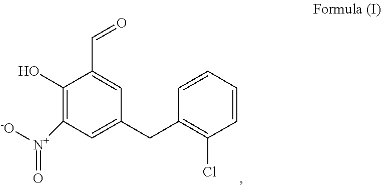

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

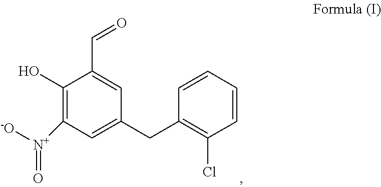

and 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises a compound of Formula I:

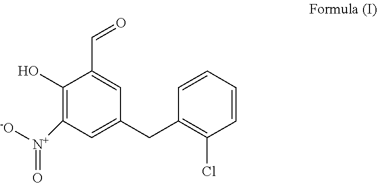

and 4-amino-1-[(2R,3R,4S,5R)-3,4-dihydroxy-5-(hydroxymethyl)oxolan-2-yl]-1,3,5-triazin-2-one (azacitidine) and/or 4-amino-1-[(2R,3S,4S,5R)-3,4-dihydroxy-5-(hydroxymethyl)oxolan-2-yl]pyrimidin-2-one (cytarabine) and/or 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and a compound of Formula II:

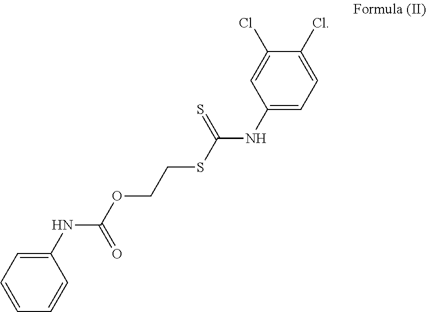 [SMILES: O=C(Nc1ccccc1)OCCSC(=S)Nc1ccc(Cl)c(Cl)c1]

In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula II:

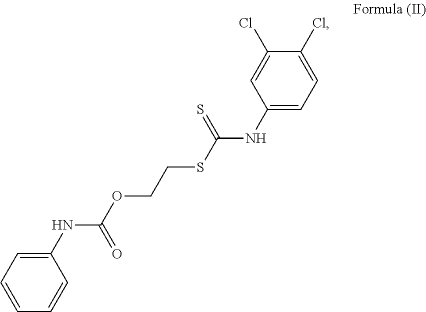 [SMILES: O=C(Nc1ccccc1)OCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) a compound of Formula II:

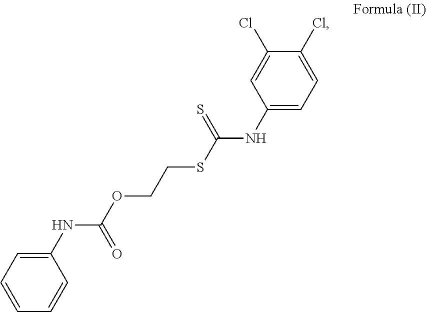 [SMILES: O=C(Nc1ccccc1)OCCSC(=S)Nc1ccc(Cl)c(Cl)c1]

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula II:

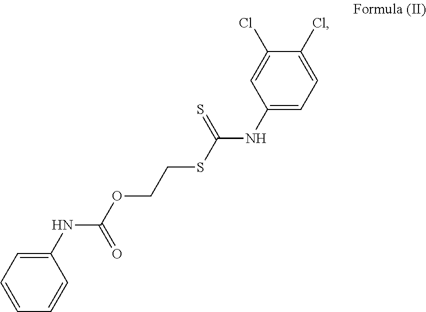 [SMILES: O=C(Nc1ccccc1)OCCSC(=S)Nc1ccc(Cl)c(Cl)c1]

and 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises a compound of Formula II:

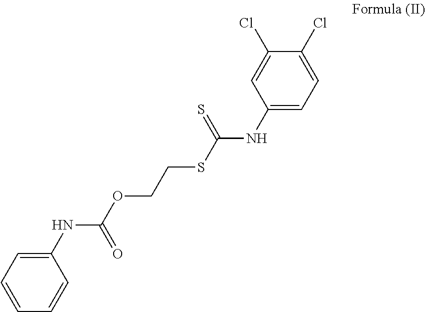 [SMILES: O=C(Nc1ccccc1)OCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine) and/or 4-amino-1-[(2R,3S,4S,5R)-3,4-dihydroxy-5-(hydroxymethyl)oxolan-2-yl]pyrimidin-2-one (cytarabine) and/or 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula I:

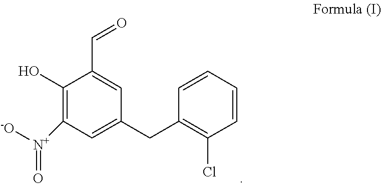

In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

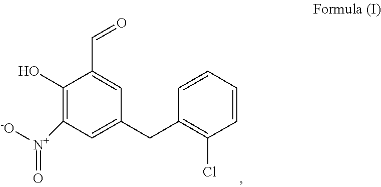

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

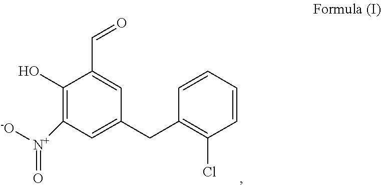

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

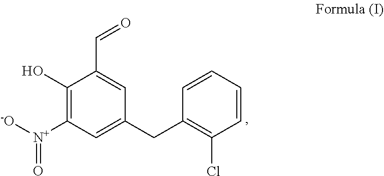

and 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

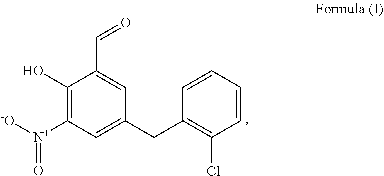

and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula II:

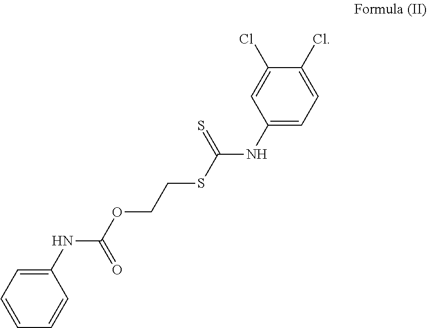 [SMILES: O=C(Nc1ccccc1)OCCSC(=S)Nc1ccc(Cl)c(Cl)c1]

In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

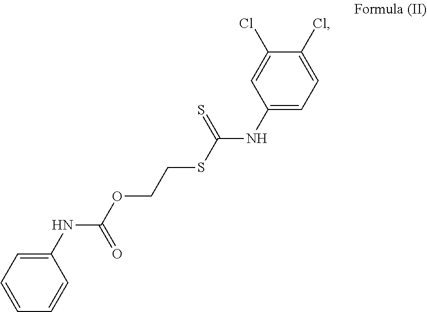 [SMILES: O=C(Nc1ccccc1)OCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

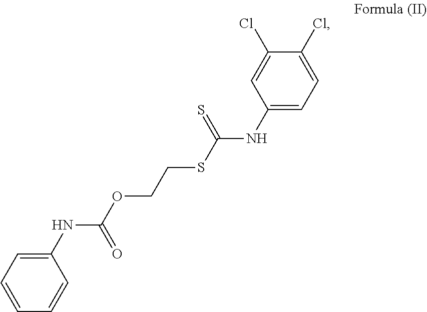 [SMILES: O=C(Nc1ccccc1)OCCSC(=S)Nc1ccc(Cl)c(Cl)c1]

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

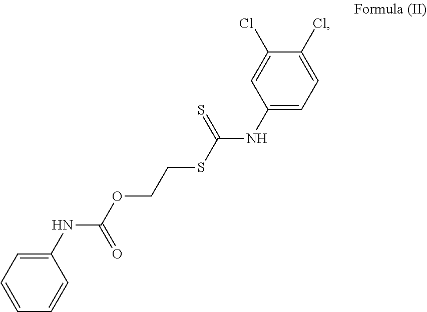 [SMILES: O=C(Nc1ccccc1)OCCSC(=S)Nc1ccc(Cl)c(Cl)c1]

and 4-amino-1-[(2R,4S,5R)-4-hydroxy-5-(hydroxymethyl)oxolan-2-yl]-1,3,5-triazin-2-one (decitabine). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

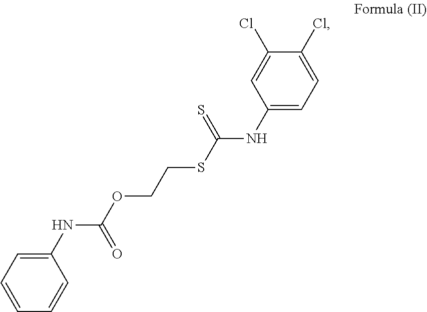 [SMILES: O=C(Nc1ccccc1)OCCSC(=S)Nc1ccc(Cl)c(Cl)c1]

and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845) and a compound of Formula I:

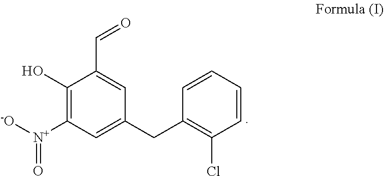

In some embodiments, this combination therapeutic administered to the subject having cancer further comprises a CDK9 inhibitor and/or a MCL-1 inhibitor.

In some embodiments, the combination therapeutic administered to the subject having cancer comprises 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991) and a compound of Formula I:

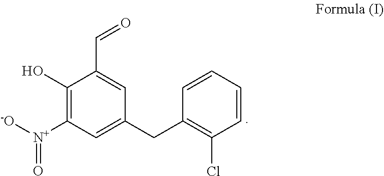

In some embodiments, this combination therapeutic administered to the subject having cancer further comprises a CDK9 inhibitor and/or a MCL-1 inhibitor.

In some embodiments, the combination therapeutic administered to the subject having cancer comprises (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845) and a compound of Formula II:

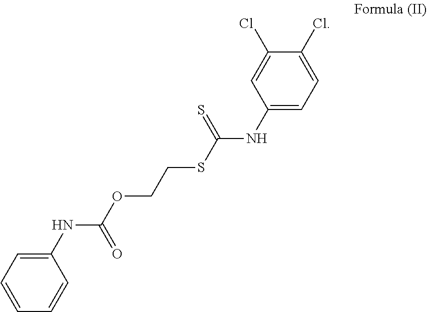 [SMILES: O=C(Nc1ccccc1)OCCSC(=S)Nc1ccc(Cl)c(Cl)c1]

In some embodiments, this combination therapeutic administered to the subject having cancer further comprises a CDK9 inhibitor and/or a MCL-1 inhibitor.

In some embodiments, the combination therapeutic administered to the subject having cancer comprises v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991) and a compound of Formula II:

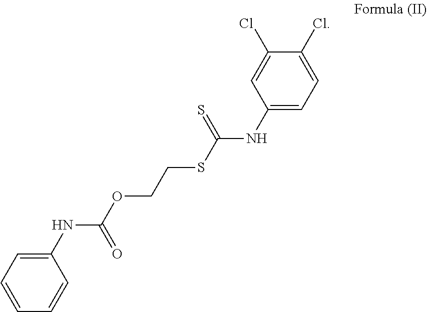 [SMILES: O=C(Nc1ccccc1)OCCSC(=S)Nc1ccc(Cl)c(Cl)c1]

In some embodiments, this combination therapeutic administered to the subject having cancer further comprises a CDK9 inhibitor and/or a MCL-1 inhibitor.

Another aspect of the present application is directed to a method of treating cancer in a subject that involves selecting a subject having cancer and administering to said subject a combination therapeutic comprising a BH3 protein mimetic and an agent that blocks autophagy.

Subjects to be treated in accordance with this aspect of the disclosure include subjects having cancer, particularly a cancer that is resistant to treatment with apoptosis inducing drug, a form of cancer susceptible to becoming resistant to treatment with apoptosis inducing drug, e.g., resistant to treatment with a BH3 mimetic. Suitable cancers to be treated in accordance with this aspect of the disclosure are disclosed supra.

In some embodiments, a subject treated in accordance with this aspect of the disclosure is one that has or is at risk of having a drug resistant form of leukemia, e.g., acute myeloid leukemia, T cell acute lymphocytic leukemia (T-ALL), early T cell progenitor leukemia (ETP-ALL), or chronic lymphocytic leukemia. In some embodiments, the leukemia is resistant to treatment with a BH3 protein mimetic

Suitable BH3 protein mimetics for use in the combination therapeutic in accordance with this aspect of the disclosure include, e.g., navitoclax, venetoclax, S55746, BLC201, S63845, AMG-176, AZD-5991, ABT-737, AT101, A-1331852, A-1155463, TW-37, A-1210477, and Sabutoclax, as described supra.

In embodiments of the various aspects of the present application, the agent that blocks autophagy in the combination therapeutic is an endosomal acidification inhibitor and/or deacidifier, a PI3K inhibitor, a MAPK inhibitor, a mitophagy inhibitor, a VPS34 inhibitor, ATG4B inhibitor, USP10 and/or USP13 inhibitor, or combinations thereof.

In some embodiments, the autophagy inhibitor of the combination therapeutic is an endosomal acidification inhibitor and/or deacidifier. Suitable endosomal acidification inhibitors and/or deacidifiers include, without limitation, N′-(7-chloroquinolin-4-yl)-N,N-diethylpentane-1,4-diamine (chloroquine), 2-[4-[(7-chloroquinolin-4-yl)amino]pentyl-ethylamino]ethanol (hydroxychloroquine); N-(7-chloroquinolin-4-yl)-N′-[6-[(7-chloroquinolin-4-yl)amino]hexyl]-N′-methylhexane-1,6-diamine (DC661), (3Z,5E,7R,8S,9S,11E,13E,15S,16R)-16-[(2S,3R,4S)-4-[(2R,4R,5S,6R)-2,4-dihydroxy-5-methyl-6-propan-2-yloxan-2-yl]-3-hydroxypentan-2-yl]-8-hydroxy-3,15-dimethoxy-5,7,9,11-tetramethyl-1-oxacyclohexadeca-3,5,11,13-tetraen-2-one (Bafilomycin A1), N-(7-chloroquinolin-4-yl)-N′-[2-[(7-chloroquinolin-4-yl)amino]ethyl]-N′-methylethane-1,2-diamine;trihydrochloride (Lys05), or derivatives thereof.

In some embodiments, the autophagy inhibitor of the combination inhibitor is a P3K inhibitor. Suitable PI3K inhibitors include, without limitation, 3-methyl-7H-purin-6-imine (3-Methyladenin), 2-morpholin-4-yl-8-phenylchromen-4-one (LY294002), or derivatives thereof.

In some embodiments, the autophagy inhibitor of the combination therapeutic is a MAPK inhibitor. Suitable MAPK inhibitors include, without limitation, 4-[5-(4-fluorophenyl)-4-(pyridin-4-yl)-1H-imidazol-2-yl]phenol (SB202190), 4-[4-(4-fluorophenyl)-2-(4-methanesulfinylphenyl)-1H-imidazol-5-yl]pyridine (SB203580), or derivatives thereof.

In some embodiments, the autophagy inhibitor of the combination therapeutic is a mitphagy inhibitor. Suitable mitophagy inhibitors include, without limitation, the Drp-1 inhibitor 3-(2,4-dichloro-5-methoxyphenyl)-2-sulfanylidene-1H-quinazolin-4-one (Mdivi-1), or derivative thereof.

In some embodiments, the autophagy inhibitor of the combination inhibitor is a ATG4B inhibitor. Suitable ATG4B inhibitors include, without limitation N-pyridin-2-ylpyridine-2-carbothioamide (NSC 185058), 3-[[4-[(E)-2-(7-chloroquinolin-4-yl)ethenyl]phenyl]-(2-phenylethylsulfanyl)methyl]sulfanylpropanoic acid (LV-320), or derivatives thereof.

In some embodiments, the autophagy inhibitor of the combination inhibitor is a USP10 and/or USP13 inhibitor. Suitable USP10 and/or USP13 inhibitors include, without limitation, 6-fluoro-N-[(4-fluorophenyl)methyl]quinazolin-4-amine (Spautin-1), or derivative thereof.

In some embodiments, administering comprises administering the BH3 protein mimetic and the agent that blocks autophagy of the combination therapeutic to the subject concurrently. In other embodiments, administering comprises administering the agent that blocks autophagy to the subject having cancer prior to the BH3 protein mimetic.

In some embodiments, the combination therapeutic further comprises a chemotherapeutic drug. Suitable chemotherapeutic drugs include hypomethylating agents as described supra.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(I H-pyrrolo[2,3-b]pyridin-5-yloxy)benzamide (venetoclax), and N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,3R,4S,5R)-3,4-dihydroxy-5-(hydroxymethyl)oxolan-2-yl]-1,3,5-triazin-2-one (azacitidine), and N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,3S,4S,5R)-3,4-dihydroxy-5-(hydroxymethyl)oxolan-2-yl]pyrimidin-2-one (cytarabine) and N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,4S,5R)-4-hydroxy-5-(hydroxymethyl)oxolan-2-yl]-1,3,5-triazin-2-one (decitabine) and N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic administered to the subject having cancer comprises N′-(7-chloroquinolin-4-yl)-N,N-diethylpentane-1,4-diamine (chloroquine) and +(3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176). In some embodiments, this combination therapeutic administered to the subject having cancer further comprises a CDK9 inhibitor and/or a MCL-1 inhibitor.

In some embodiments, the combination therapeutic administered to the subject having cancer comprises N′-(7-chloroquinolin-4-yl)-N,N-diethylpentane-1,4-diamine (chloroquine), (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises N′-(7-chloroquinolin-4-yl)-N,N-diethylpentane-1,4-diamine (chloroquine) and (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845). In some embodiments, this combination therapeutic administered to the subject having cancer further comprises a CDK9 inhibitor and/or a MCL-1 inhibitor.

In some embodiments, the combination therapeutic administered to the subject having cancer comprises N′-(7-chloroquinolin-4-yl)-N,N-diethylpentane-1,4-diamine (chloroquine) and v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991). In some embodiments, this combination therapeutic administered to the subject having cancer further comprises a CDK9 inhibitor and/or a MCL-1 inhibitor.

In some embodiments, the combination therapeutic administered to the subject having cancer comprises 3-(2,4-dichloro-5-methoxyphenyl)-2-sulfanylidene-1H-quinazolin-4-one (Mdivi-1). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 3-(2,4-dichloro-5-methoxyphenyl)-2-sulfanylidene-1H-quinazolin-4-one (Mdivi-1). In some embodiments, this combination therapeutic administered to the subject having cancer further comprises a CDK9 inhibitor and/or a MCL-1 inhibitor.

In some embodiments, the combination therapeutic administered to the subject having cancer comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 3-(2,4-dichloro-5-methoxyphenyl)-2-sulfanylidene-1H-quinazolin-4-one (Mdivi-1), and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises 3-(2,4-dichloro-5-methoxyphenyl)-2-sulfanylidene-1H-quinazolin-4-one (Mdivi-1), and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises 3-(2,4-dichloro-5-methoxyphenyl)-2-sulfanylidene-1H-quinazolin-4-one (Mdivi-1), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661) and (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-136-thia-1,14-diazatetracyclo[14.7.2.03,6.019,24]pentacosa-8,16(25),17,19(24)-tetraene]-15′-one (AMG-176). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661) and (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845). In some embodiments, this combination therapeutic administered to the subject having cancer further comprises a CDK9 inhibitor and/or a MCL-1 inhibitor.

In some embodiments, the combination therapeutic administered to the subject having cancer comprises N-(7-chloroquinolin-4-yl)-N′-[6-[(7-chloroquinolin-4-yl)amino]hexyl]-N′-methylhexane-1,6-diamine (DC661) and v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991). In some embodiments, this combination therapeutic administered to the subject having cancer further comprises a CDK9 inhibitor and/or a MCL-1 inhibitor.

In accordance with the methods described herein, administration of the agent(s) alone or in combination is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of cancer to be treated.

A therapeutically effective amount of the therapeutic agent(s) alone or in combination in the methods disclosed herein is an amount that, when administered over a particular time interval, results in increased sensitivity to treatment with the apoptosis inducing drug or reverses resistance to treatment with the apoptosis inducing drug, which further leads to a slowing or halting of cancer growth, cancer regression, cessation of symptoms, etc. The therapeutic agents for use in the presently disclosed methods may be administered to a subject one time or multiple times. In those embodiments where the therapeutic agents are administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer, e.g., particularly drug resistance, or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the therapeutic agents administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

The effectiveness of the methods of the present application in increasing cancer cell sensitivity to treatment with the apoptosis inducing drug or reversing drug resistance of the cancer may be evaluated, for example, by assessing changes in cancer burden and/or disease progression following treatment with the therapeutic agents as described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). In some embodiments, cancer burden and/or disease progression is evaluated using imaging techniques including, e.g., X-ray, computed tomography (CT) scan, magnetic resonance imaging, multiparametric ultrasound (mpUS), multiparametric magnetic resonance imaging (mpMRI), and nuclear imaging (positron emission tomography [PET]) (Eisenhauer et al., “New Response Evaluation Criteria in Solid Tumours: Revised RECIST Guideline (Version 1.1),” Eur. J. Cancer 45(2): 228-247 (2009), which is hereby incorporated by reference in its entirety). Cancer regression or progression may be monitored prior to, during, and/or following treatment with one or more of the therapeutic agents described herein.

In some embodiments, the response to treatment with the methods described herein results in at least about 1%, 2%, 3%, 4%, 5%, 6%, 7%, 8%, 9%, 10%, 20%, 30%, 40%, 50%, 60%, 70%, 80%, 90%, 95%, 99%, or 100% increase in sensitivity to treatment with the apoptosis inducing drug, and thus increase in cancer cell apoptosis. Thus, the response to treatment with any of the methods described herein may be partial (e.g., at least a 30% increase in cancer cell apoptosis, as compared to baseline cancer cells treated with apoptosis inducing drug only) or complete (elimination of the tumor or circulating cancer cells).

In some embodiments, the effectiveness of the methods described herein may be evaluated, for example, by assessing cancer cell apoptosis following treatment with the combination therapeutics described herein. In some embodiments, the methods described herein, by increasing cancer cell sensitivity to drug induced apoptosis may be effective to inhibit disease progression, inhibit cancer growth/spread, relieve cancer-related symptoms, inhibit tumor-secreted factors (e.g., tumor-secreted hormones), delay the appearance of primary or secondary cancer tumors, slow development of primary or secondary cancer tumors, decrease the occurrence of primary or secondary cancer tumors, slow or decrease the severity of secondary effects of disease, arrest tumor growth, and/or achieve regression of cancer in a selected subject. Thus, the methods described herein are effective to increase the therapeutic benefit to the selected subject.

In some embodiments, the methods described herein reduce the rate of cancer growth in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In certain embodiments, the methods described herein reduce the rate of cancer invasiveness in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In specific embodiments, the methods described herein reduce the rate of cancer progression in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In various embodiments, the methods described herein reduce the rate of cancer recurrence in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more. In some embodiments, the methods described herein reduce the rate of metastasis in the selected subject by at least about 1%, 2%, 3%, 4%, 5%, 6%, 7%, 8%, 9%, 10%, 20%, 30%, 40%, 50%, 60%, 70%, 80%, 90%, 95%, 99%, or more.

Another aspect of the present application is directed to combination therapeutics. A first combination therapeutic of the disclosure comprises an agent that modulates mitochondrial structure and an apoptosis inducing drug. In some embodiments, the combination therapeutic further comprises a chemotherapeutic drug. Suitable agents that modulate mitochondrial structure, apoptosis inducing drugs, and chemotherapeutic drugs are described supra. A second combination therapeutic of the disclosure comprises an autophagy inhibitor and an apoptosis inducing drug. In some embodiments, this combination therapeutic further comprises a chemotherapeutic drug. Suitable autophagy inhibitors, apoptosis inducing drugs, and chemotherapeutic drugs are described supra.

In some embodiments, the components of the combination therapeutics are formulated together in a single pharmaceutical composition. In other embodiments, the components of the combination therapeutic are formulated as separate pharmaceutical compositions and provided for administration together.

In some embodiments, the combination therapeutic as described herein provides a synergistic effect, as measured by, for example, the extent of the response (e.g., cancer cell apoptosis), the response rate, the time to disease progression, or the survival period, as compared to the effect achievable on dosing with the apoptosis inducing drug, alone at its conventional dose, or as compared to the combination therapeutic formulated without the agent that modulates mitochondrial structure and/or the agent that blocks autophagy. For example, the effect of the combination treatment is synergistic if a beneficial effect is obtained in a patient that does not respond (or responds poorly) to the apoptosis inducing drug alone. In addition, the effect of the combination treatment is defined as affording a synergistic effect if the apoptosis inducing drug is administered at dose lower than its conventional dose and the therapeutic effect, as measured by, for example, the extent of the response, the response rate, the time to disease progression or the survival period, is equivalent to that achievable on dosing conventional amounts of the apoptosis inducing drug. In particular, synergy is deemed to be present if the conventional dose of the apoptosis inducing drug is reduced without detriment to one or more of the extent of the response, the response rate, the time to disease progression, and survival data, in particular without detriment to the duration of the response, but with fewer and/or less troublesome side-effects than those that occur when conventional doses of each component are used.

In one embodiment, the combination therapeutic encompasses an agent that modules mitochondrial structure and/or an agent that blocks autophagy and together with one or more chemotherapeutic drugs (including, for example, an apoptosis inducing drug and/or a hypomethylating agent) formulated separately, but for administration together. In another embodiment, the combination therapeutic encompasses the agent(s) and one or more chemotherapeutic drugs formulated together in a single formulation. A single formulation refers to a single carrier or vehicle formulated to deliver effective amounts of both therapeutic agents in a unit dose to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension. In yet another embodiment, the vehicle is a nanodelivery vehicle.

Suitable nanodelivery vehicles are known in the art and include, for example and without limitation, nanoparticles such as albumin particles (Hawkins et al., “Protein nanoparticles as drug carriers in clinical medicine,” Advanced Drug Delivery Reviews 60(8): 876-885 (2008), which is hereby incorporated by reference in its entirety), cationic bovine serum albumin nanoparticles (Han et al., “Cationic bovine serum albumin based self-assembled nanoparticles as siRNA delivery vector for treating lung metastasis cancer,” Small 10(3): (2013), which is hereby incorporated by reference in its entirety), gelatin nanoparticles (Babaei et al., “Fabrication and evaluation of gelatine nanoparticles for delivering of anti-cancer drug,” Int'l J. NanoSci. Nanotech. 4:23-29 (2008), which is hereby incorporated by reference in its entirety), gliadin nanoparticles (Gulfam et al., “Anticancer drug-loaded gliadin nanoparticles induced apoptosis in breast cancer cells,” Langmuir 28: 8216-8223 (2012), which is hereby incorporated by reference in its entirety), zein nanoparticles (Aswathy et al., “Biocompatible fluorescent zein nanoparticles for simultaneous bioimaging and drug delivery application,”Advances in Natural Sciences: Nanoscience and Nanotechnology 3(2) (2012), which is hereby incorporated by reference in its entirety), and casein nanoparticles (Elzoghby et al., “Ionically-crosslinked milk protein nanoparticles as flutamide carriers for effective anticancer activity in prostate cancer-bearing rats,” Eur. J. Pharm. Biopharm. 85(3): 444-451 (2013) which is hereby incorporated by reference in its entirety); liposomes (Feldman et al., “First-in-man study of CPX-351: a liposomal carrier containing cytarabine and daunorubicin in a fixed 5:1 molar ratio for the treatment of relapsed and refractory acute myeloid leukemia,” J. Clin. Oncol. 29(8): 979-985 (2011); Ong et al., “Development of stealth liposome coencapsulating doxorubicin and fluoxetine,” J. Liposome Res. 21(4): 261-271 (2011); and Sawant et al., “Palmitoyl ascorbate-modified liposomes as nanoparticle platform for ascorbate-mediated cytotoxicity and paclitaxel co-delivery,” Eur. J. Pharm. Biopharm. 75(3): 321-326 (2010), which are hereby incorporated by reference in their entirety); polymeric nanoparticles, including synthetic polymers, such as poly-ε-caprolactone, polyacrylamine, and polyacrylate, and natural polymers, such as, e.g., albumin, gelatin, or chitosan (Agnihotri et al., “Novel interpenetrating network chitosan-poly(ethylene oxide-g-acrylamide)hydrogel microspheres for the controlled release of capecitabine,” Int J Pharm 324: 103-115 (2006); Bilensoy et al., “Intravesical cationic nanoparticles of chitosan and polycaprolactone for the delivery of Mitomycin C to bladder tumor,” Int J Pharm 371: 170-176 (2009), which are hereby incorporated by reference); dendrimer nanocarriers (e.g., poly(amido amide) (PAMAM)) (Han et al., “Peptide conjugated PAMAM for targeted doxorubicin delivery to transferrin receptor overexpressed tumors,” Mol Pharm 7: 2156-2165 (2010); and Singh et al., “Folate and Folate-PEG-PAMAM dendrimers: synthesis, characterization, and targeted anticancer drug delivery potential in tumor bearing mice,” Bioconjugate Chem 19, 2239-2252 (2008), which are hereby incorporated by reference in their entirety); silica nanoparticle (e.g., xerogels and mesoporous silica nanoparticles) (He et al., “A pH-responsive mesoporous silica nanoparticles based multi-drug delivery system for overcoming multidrug resistance,” Biomaterials 32: 7711-7720 (2011); Prokopowicz M., “Synthesis and in vitro characterization of freeze-dried doxorubicin-loaded silica xerogels,” J Sol-Gel Sci Technol 53: 525-533 (2010); Maver et al., “Novel hybrid silica xerogels for stabilization and controlled release of drug,” Int J Pharm 330:164-174 (2007), which are hereby incorporated by reference in their entirety).

The agents and combination therapeutics described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is incorporated herein by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein, i.e., agents that modulate mitochondrial structure, apoptosis inducing drugs, autophagy inhibitors, includes any analog, derivative, isomer, metabolite, pharmaceutically acceptable salt, pharmaceutical product, hydrate, N-oxide, crystal, polymorph, prodrug or any combination thereof. In certain embodiments, the therapeutic agents disclosed herein may be in a prodrug form, meaning that it must undergo some alteration (e.g., oxidation or hydrolysis) to achieve its active form.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and a compound of Formula I:

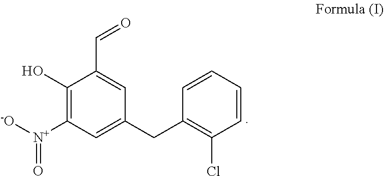

In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

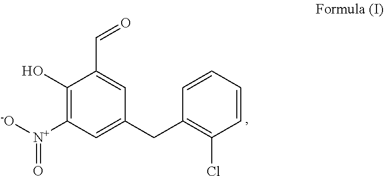

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

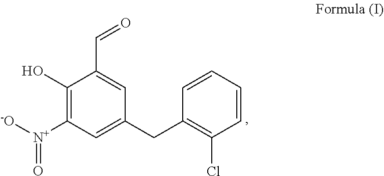

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula I:

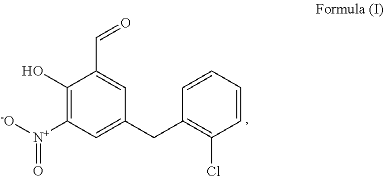

and 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises a compound of Formula I:

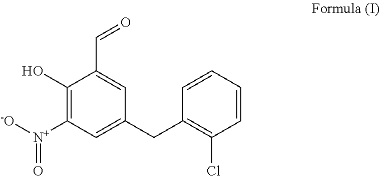

and 4-amino-1-[(2R,3R,4S,5R)-3,4-dihydroxy-5-(hydroxymethyl)oxolan-2-yl]-1,3,5-triazin-2-one (azacitidine) and/or 4-amino-1-[(2R,3S,4S,5R)-3,4-dihydroxy-5-(hydroxymethyl)oxolan-2-yl]pyrimidin-2-one (cytarabine) and/or 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and a compound of Formula II:

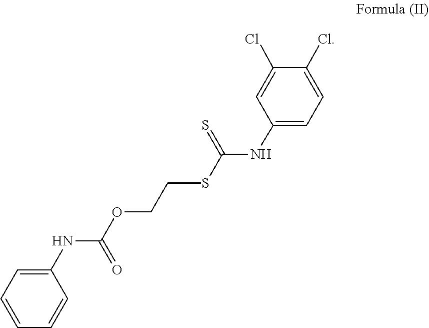 [SMILES: O=C(Nc1ccccc1)OCCCSC(=S)Nc1ccc(Cl)c(Cl)c1]

In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula II:

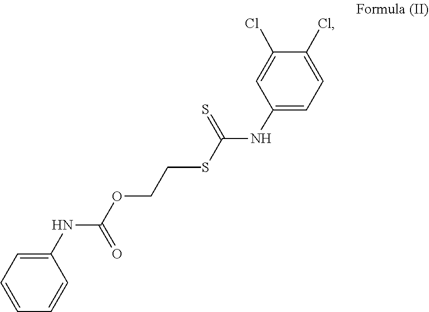 [SMILES: O=C(Nc1ccccc1)OCCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula II:

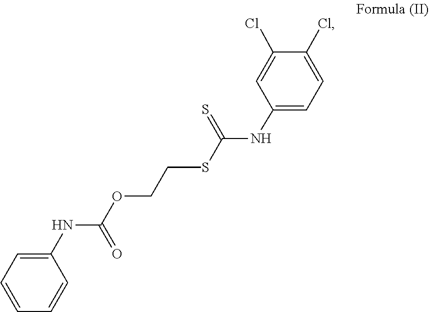 [SMILES: O=C(Nc1ccccc1)OCCCSC(=S)Nc1ccc(Cl)c(Cl)c1]

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), a compound of Formula H:

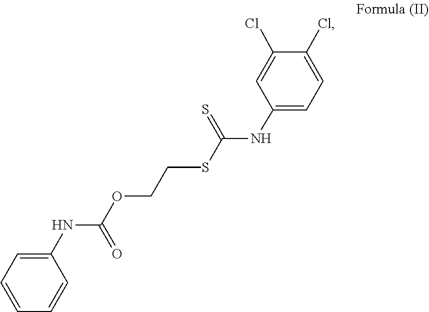 [SMILES: O=C(Nc1ccccc1)OCCCSC(=S)Nc1ccc(Cl)c(Cl)c1]

and 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises a compound of Formula H:

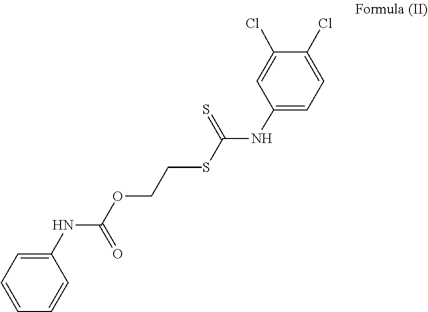 [SMILES: O=C(Nc1ccccc1)OCCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine) and/or 4-amino-1-[(2R,3S,4S,5R)-3,4-dihydroxy-5-(hydroxymethyl)oxolan-2-yl]pyrimidin-2-one (cytarabine) and/or 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula I:

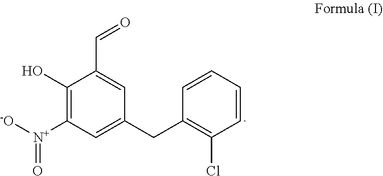

In some embodiments, this combination therapeutic further comprises a CDK9 inhibitor and/or a MCL-1 inhibitor.

In some embodiments, the combination therapeutic comprises (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

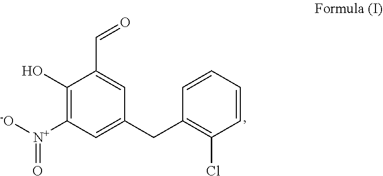

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

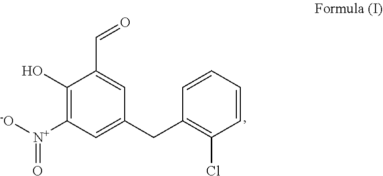

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

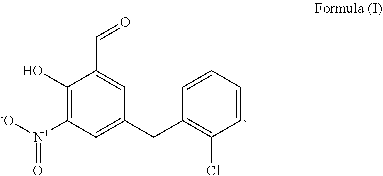

and 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula I:

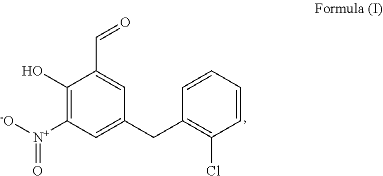

and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula II:

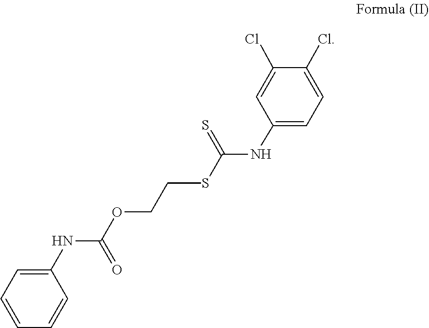 [SMILES: O=C(Nc1ccccc1)OCCCSC(=S)Nc1ccc(Cl)c(Cl)c1]

In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

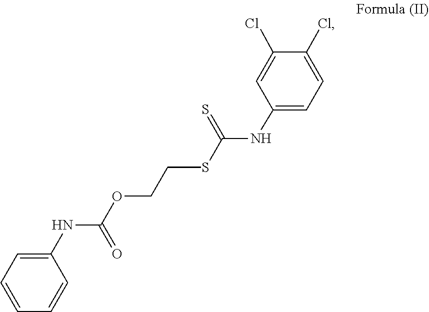 [SMILES: O=C(Nc1ccccc1)OCCCSC(=S)Nc1ccc(Cl)c(Cl)c1]

and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

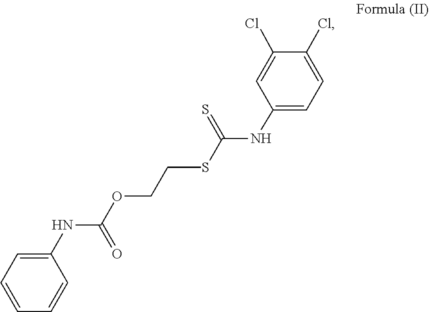 [SMILES: O=C(Nc1ccccc1)OCCCSC(=S)Nc1ccc(Cl)c(Cl)c1]

and 4-amino-1-[(2R,3S,4S,5R)-3,4-dihydroxy-5-(hydroxymethyl)oxolan-2-yl]pyrimidin-2-one (cytarabine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

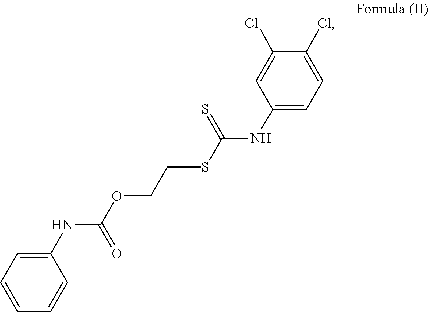 [SMILES: O=C(Nc1ccccc1)OCCCSC(=S)Nc1ccc(Cl)c(Cl)c1]

and 4-amino-1-[(2R,4S,5R)-4-hydroxy-5-(hydroxymethyl)oxolan-2-yl]-1,3,5-triazin-2-one (decitabine). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), a compound of Formula II:

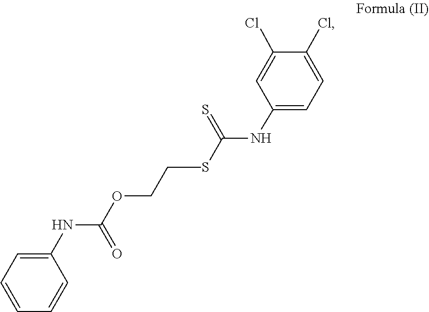 [SMILES: O=C(Nc1ccccc1)OCCCSC(=S)Nc1ccc(Cl)c(Cl)c1]

and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845) and a compound of Formula I:

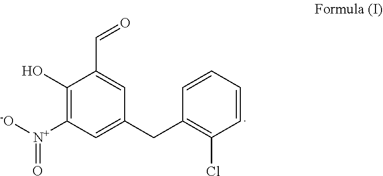

In some embodiments, this combination therapeutic further comprises a CDK9 inhibitor and/or a MCL-1 inhibitor.

In some embodiments, the combination therapeutic comprises 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991) and a compound of Formula I:

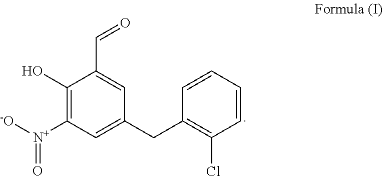

In some embodiments, this combination therapeutic further comprises a CDK9 inhibitor and/or a MCL-1 inhibitor.

In some embodiments, the combination therapeutic comprises (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845) and a compound of Formula II:

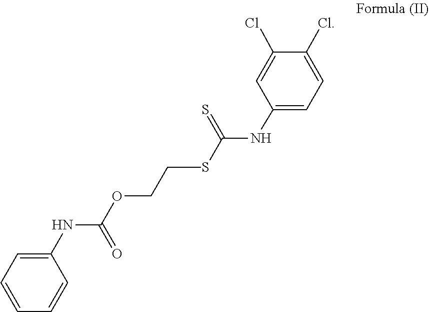 [SMILES: O=C(Nc1ccccc1)OCCCSC(=S)Nc1ccc(Cl)c(Cl)c1]

In some embodiments, this combination therapeutic further comprises a CDK9 inhibitor and/or a MCL-1 inhibitor.

In some embodiments, the combination therapeutic comprises v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991) and a compound of Formula II:

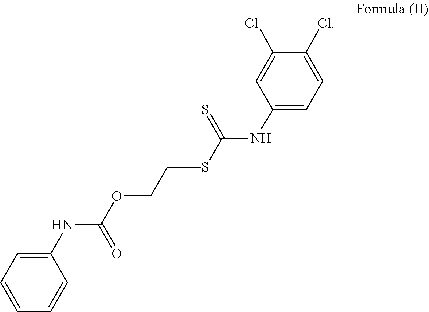 [SMILES: O=C(Nc1ccccc1)OCCCSC(=S)Nc1ccc(Cl)c(Cl)c1]

In some embodiments, this combination therapeutic further comprises a CDK9 inhibitor and/or a MCL-1 inhibitor.

In some embodiments, the combination therapeutic administered to the subject having cancer comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(I H-pyrrolo[2,3-b]pyridin-5-yloxy)benzamide (venetoclax), and N′-(7-chloroquinolin-4-yl)-N,N-diethylpentane-1,4-diamine (chloroquine). In some embodiments, this combination therapeutic administered to the subject having cancer further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,3R,4S,5R)-3,4-dihydroxy-5-(hydroxymethyl)oxolan-2-yl]-1,3,5-triazin-2-one (azacitidine), and N′-(7-chloroquinolin-4-yl)-N,N-diethylpentane-1,4-diamine (chloroquine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,3S,4S,5R)-3,4-dihydroxy-5-(hydroxymethyl)oxolan-2-yl]pyrimidin-2-one (cytarabine) and N′-(7-chloroquinolin-4-yl)-N,N-diethylpentane-1,4-diamine (chloroquine). In some embodiments, this combination therapeutic further comprises a CDK9 inhibitor and/or a MCL-1 inhibitor.

In some embodiments, the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,4S,5R)-4-hydroxy-5-(hydroxymethyl)oxolan-2-yl]-1,3,5-triazin-2-one (decitabine) and N′-(7-chloroquinolin-4-yl)-N,N-diethylpentane-1,4-diamine (chloroquine). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises N′-(7-chloroquinolin-4-yl)-N,N-diethylpentane-1,4-diamine (chloroquine). In some embodiments, this combination therapeutic further comprises a CDK9 inhibitor and/or a MCL-1 inhibitor.

In some embodiments, the combination therapeutic comprises N′-(7-chloroquinolin-4-yl)-N,N-diethylpentane-1,4-diamine (chloroquine) and +(3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176). In some embodiments, this combination therapeutic further comprises a CDK9 inhibitor and/or a MCL-1 inhibitor.

In some embodiments, the combination therapeutic comprises N′-(7-chloroquinolin-4-yl)-N,N-diethylpentane-1,4-diamine (chloroquine), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises N′-(7-chloroquinolin-4-yl)-N,N-diethylpentane-1,4-diamine (chloroquine) and (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845). In some embodiments, this combination therapeutic further comprises a CDK9 inhibitor and/or a MCL-1 inhibitor.

In some embodiments, the combination therapeutic comprises N′-(7-chloroquinolin-4-yl)-N,N-diethylpentane-1,4-diamine (chloroquine) and v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991). In some embodiments, this combination therapeutic further comprises a CDK9 inhibitor and/or a MCL-1 inhibitor.

In some embodiments, the combination therapeutic comprises 3-(2,4-dichloro-5-methoxyphenyl)-2-sulfanylidene-1H-quinazolin-4-one (Mdivi-1). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-136-thia-1,14-diazatetracyclo[14.7.2.03,6.019,24]pentacosa-8,16(25),17,19(24)-tetraene]-15′-one (AMG-176), and 3-(2,4-dichloro-5-methoxyphenyl)-2-sulfanylidene-1H-quinazolin-4-one (Mdivi-1). In some embodiments, this combination therapeutic further comprises a CDK9 inhibitor and/or a MCL-1 inhibitor.

In some embodiments, the combination therapeutic comprises (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 3-(2,4-dichloro-5-methoxyphenyl)-2-sulfanylidene-1H-quinazolin-4-one (Mdivi-1), and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises 3-(2,4-dichloro-5-methoxyphenyl)-2-sulfanylidene-1H-quinazolin-4-one (Mdivi-1), and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises 3-(2,4-dichloro-5-methoxyphenyl)-2-sulfanylidene-1H-quinazolin-4-one (Mdivi-1), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), and 4-amino-1-[(2R,3R,4S,5R)-3,4-dihydroxy-5-(hydroxymethyl)oxolan-2-yl]-1,3,5-triazin-2-one (azacitidine). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661) and (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-136-thia-1,14-diazatetracyclo[14.7.2.03,6.019,24]pentacosa-8,16(25),17,19(24)-tetraene]-15′-one (AMG-176). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661) and (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (563845). In some embodiments, this combination therapeutic further comprises a CDK9 inhibitor and/or a MCL-1 inhibitor.

In some embodiments, the combination therapeutic comprises N-(7-chloroquinolin-4-yl)-N′-[6-[(7-chloroquinolin-4-yl)amino]hexyl]-N′-methylhexane-1,6-diamine (DC661) and v17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991). In some embodiments, this combination therapeutic further comprises a CDK9 inhibitor and/or a MCL-1 inhibitor.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

### EXAMPLES

The examples below are intended to exemplify the practice of embodiments of the disclosure but are by no means intended to limit the scope thereof.

### Example 1—CRISPR Screen Identifies Synthetic Lethal Vulnerabilities for Venetoclax in AML

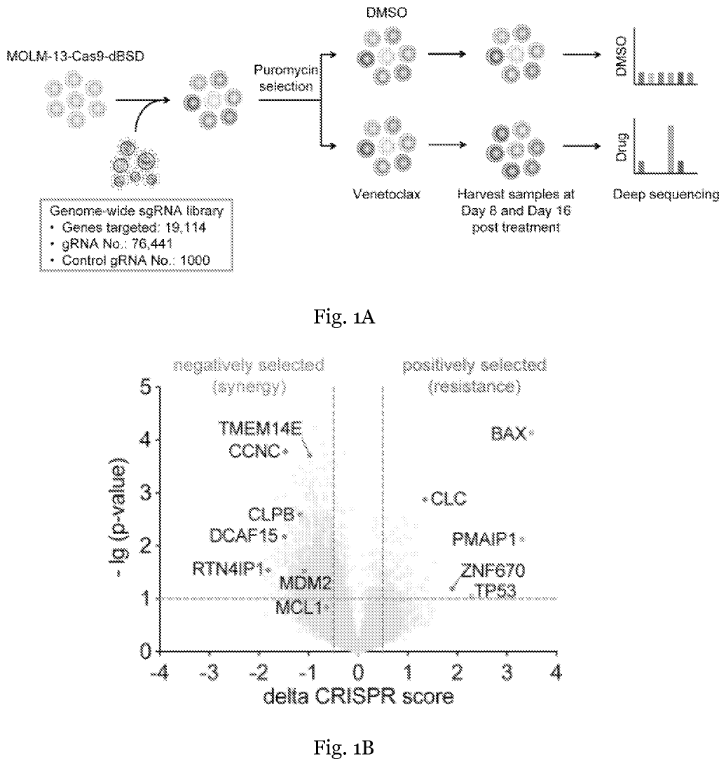

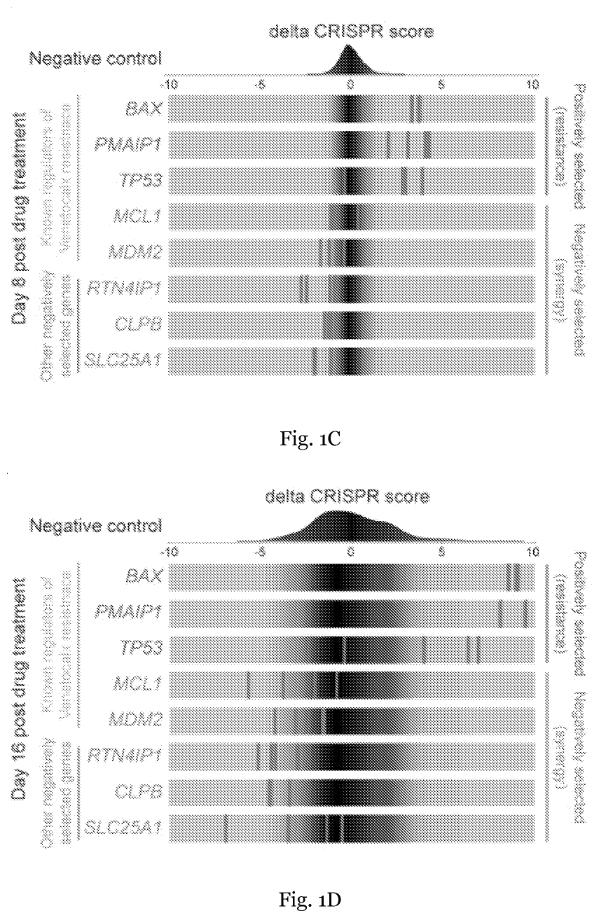

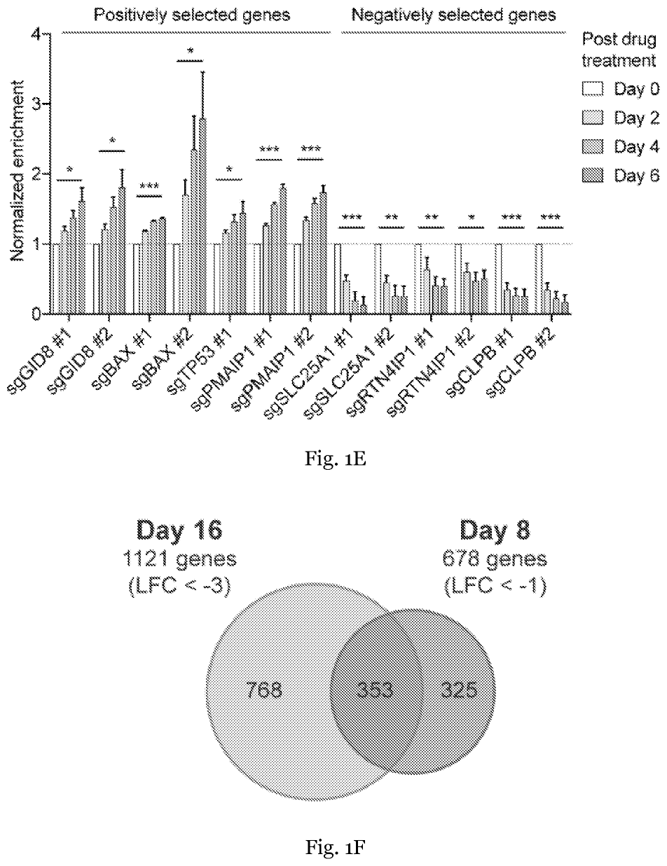

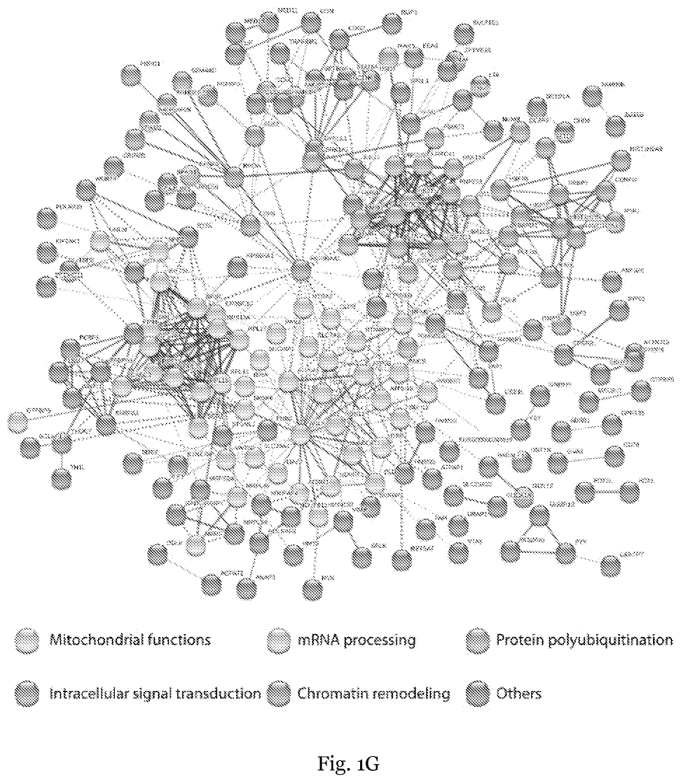

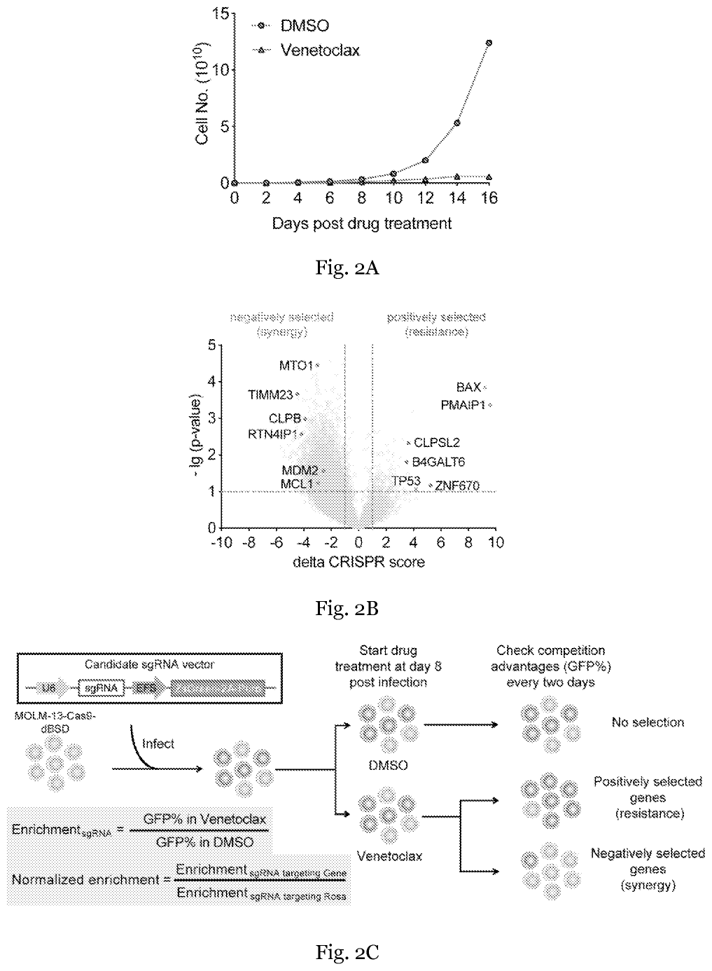

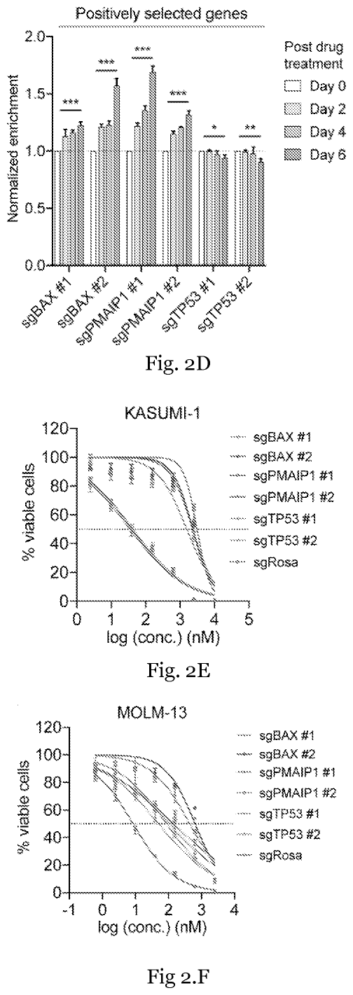

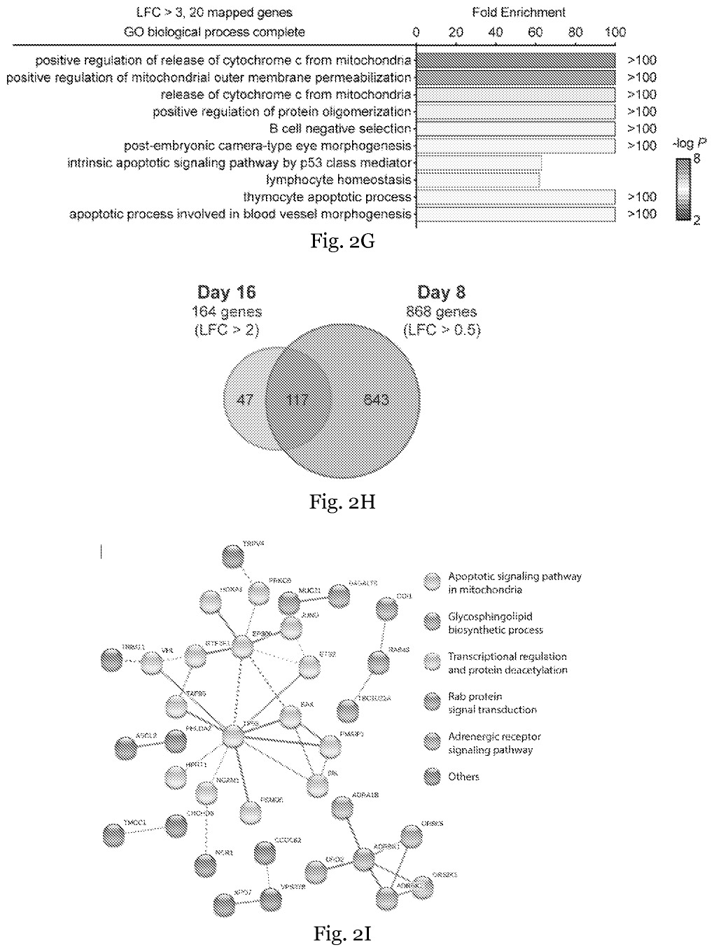

To systematically identify key factors that regulate Venetoclax resistance in human AML, a genome-wide CRISPR/Cas9 loss-of-function screen was performed (Hart et al., “High-Resolution CRISPR Screens Reveal Fitness Genes and Genotype-Specific Cancer Liabilities,” Cell 163:1515-26 (2015), which is hereby incorporated by reference in its entirety). MOLM-13 AML cells were transduced with the Brunello sgRNA library (Doench et al., “Optimized sgRNA Design to Maximize Activity and Minimize Off-Target Effects of CRISPR-Cas9,” Nat. Biotechnol. 34:184-91 (2016), which is hereby incorporated by reference in its entirety), cultured them in the presence of Venetoclax or DMSO for 16 days, and sequenced the distribution of sgRNAs at day 8 and day 16 post-drug treatment (FIG. 1A; FIG. 2A). This strategy ensured that genes that confer resistance to or synergize with Venetoclax would be positively or negatively selected, respectively, following 8 and 16 days of drug treatment compared to DMSO treatment (FIGS. 1B, 1C, 1D, 2B). BAX and PMAIP1 were among the positively selected genes (genes that their loss confers resistance), a finding consistent with the mechanism of action of the drug (Tahir et al., “Potential Mechanisms of Resistance to Venetoclax and Strategies to Circumvent it,” BMC Cancer 17:399 (2017); Karim et al., “Structural Mechanism for Regulation of Bcl-2 protein Noxa by Phosphorylation,” Sci Rep. 5:14557 (2015); and Reyna et al., “Direct Activation of BAX by BTSA1 Overcomes Apoptosis Resistance in Acute Myeloid Leukemia,” Cancer Cell 32:490-505 e10 (2017), which are each hereby incorporated by reference in their entirety). Interestingly, the tumor suppressor p53 (TP53) was also positively selected in the screen, in agreement with recent studies (Pan et al., “Synthetic Lethality of Combined Bcl-2 Inhibition and p53 Activation in AML: Mechanisms and Superior Antileukemic Efficacy,” Cancer Cell 32:748-60 e6 (2017), which is hereby incorporated by reference in its entirety), as well as the accompanying CRISPR screen performed by Tyner and colleagues (Nechiporuk et al., “The TP53 Apoptotic Network is a Primary Mediator of Resistance to BCL2 inhibition in AML Cells,” Cancer Discovery 9(7):910-925 (2019), which is hereby incorporated by reference in its entirety). MDM2, encoding an E3 ubiquitin ligase that targets p53 for degradation (Pan et al., “Synthetic Lethality of Combined Bcl-2 Inhibition and p53 Activation in AML: Mechanisms and Superior Antileukemic Efficacy,” Cancer Cell 32:748-60 e6 (2017); and Nag et al., “The MDM2-p53 Pathway Revisited,” J. Biomed Res. 27:254-71 (2013), which are each hereby incorporated by reference in their entirety) and MCL1, a primary mode of resistance to Venetoclax (Ramsey et al., “A Novel MCL1 Inhibitor Combined with Venetoclax Rescues Venetoclax-Resistant Acute Myelogenous Leukemia,” Cancer Discov. 8:1566-81 (2018); and Teh et al., “Enhancing Venetoclax Activity in Acute Myeloid Leukemia by co-Targeting MCL1,” Leukemia 32:303-12 (2018), which are each hereby incorporated by reference in their entirety) were amongst the negatively selected genes (genes that their loss synergizes with Venetoclax) at both day 8 and day 16 of drug treatment (FIGS. 1B, 1C, 1D, 2B). A number of positively and negatively selected genes (GID8, BAX, TP53, PMAIP1, SLC25A1, RTN4IP1, CLPB) were further validated throughout the screen by selecting the top sgRNAs for each gene and monitoring their ability to confer resistance to or synergize with Venetoclax in AML cells performing a competition-based assay (FIG. 2C). These results confirmed that all the sgRNAs targeting the positively selected genes demonstrated significant resistance to Venetoclax, while the sgRNAs targeting the negatively selected genes strongly sensitized MOLM-13 cells to the drug treatment (FIGS. 1E, 2D). Likewise, a significant increase of half maximal inhibitory concentration (IC₅₀) in KASUMI-1 and MOLM-13 cells upon deletion of BAX and PMAIP1 validated the gain of resistance of Venetoclax in BAX- and PMAIP1-deficient AML cells. Notably, TP53 sgRNAs increased the Venetoclax IC₅₀ in p53 wild-type MOLM-13 but not p53-mutant KASUMI-1, confirming the specificity of the guides used in this study (FIGS. 2E, 2F). Together, the genome-wide CRISPR/Cas9 loss-of-function screen successfully revealed potential modes of resistance to Venetoclax as well as synthetic lethal partners in AML.

Next, key pathways and biological processes that are enriched in the screen were identified. Gene Ontology (GO) analysis was performed, focusing firstly on positively selected genes that were at least 8-fold (LFC>3) enriched. The majority of these genes significantly clustered into key biological processes categories that regulate the intrinsic apoptotic signaling pathway, including cytochrome c release and mitochondrial outer membrane permeabilization (FIG. 2G). STRING protein-protein interaction network analysis was then performed using the positively selected genes at both day 8 and day 16 of Venetoclax treatment (FIG. 2H). One of the major clusters from this network was the p53-mediated apoptotic signaling pathway in mitochondria (FIG. 2I). The negatively selected genes were particularly interesting, as they are potential therapeutic targets for circumventing Venetoclax resistance. Gene Ontology analysis of the “sensitizer” genes revealed strong enrichment for mitochondrial processes, such as mitochondrial transport, organization and oxidative phosphorylation (FIG. 2J). Moreover, when the two time-points of the screen were combined, 353 common negatively selected genes were successfully identified (FIG. 1F). Among these, 55 were genes that encode proteins functioning in mitochondria, highlighting the relevance of mitochondrial processes in the sensitization of AML cells to Venetoclax. Consistently, mapping this 353 gene-set on the STRING database generated a highly connected network consisting mainly of genes that regulate mitochondrial functions, as well as chromatin remodelers, ubiquitin ligases, signal transducers and components of mRNA processing (FIG. 1G). Therefore, the genome-wide CRISPR/Cas9 loss-of-function screen identified mitochondrial processes as key potential synthetic lethal vulnerabilities for Venetoclax in AML.

### Example 2—Venetoclax Treatment Leads to Aberrant Mitochondrial Structure and Depolarization in AML

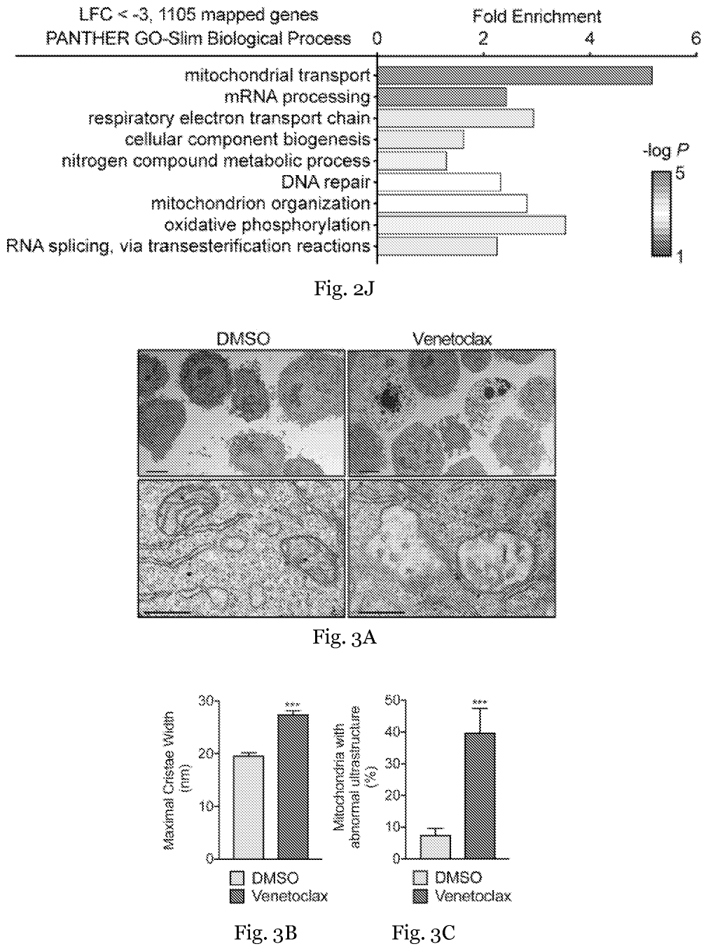

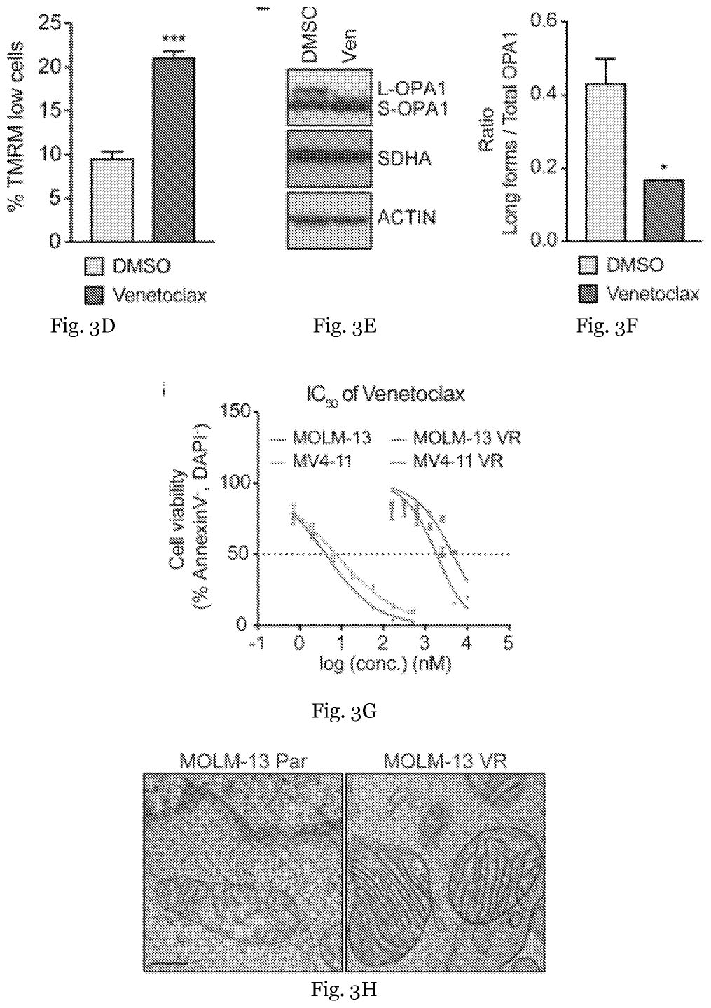

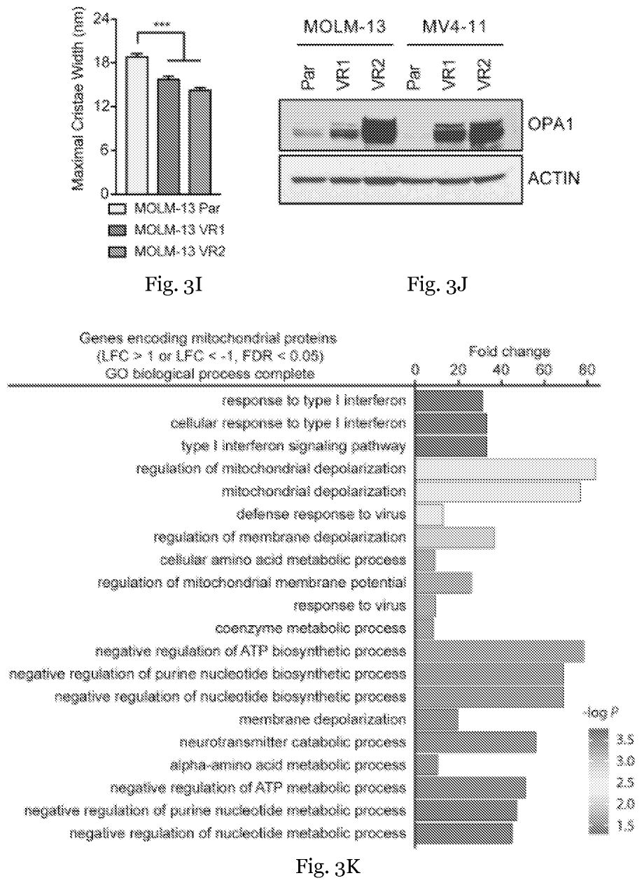

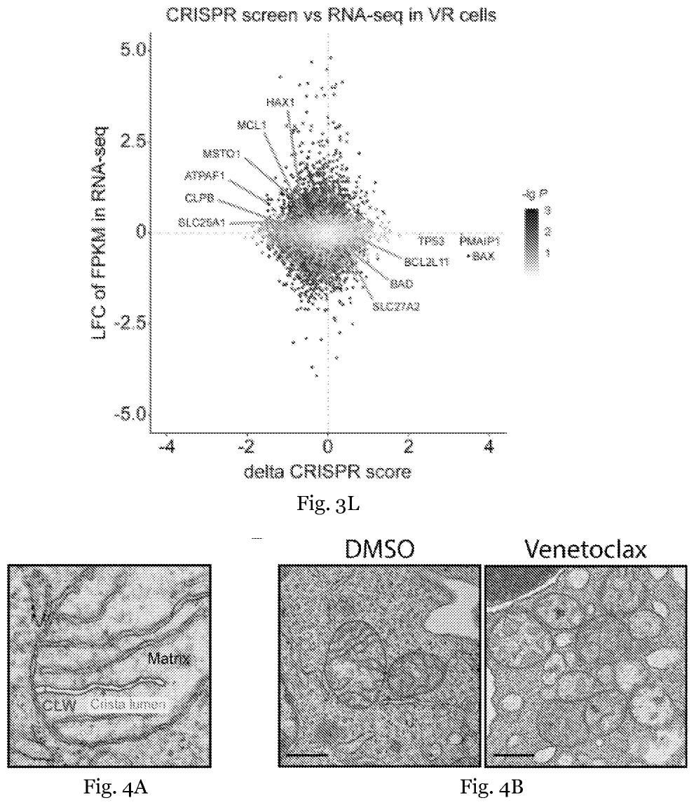

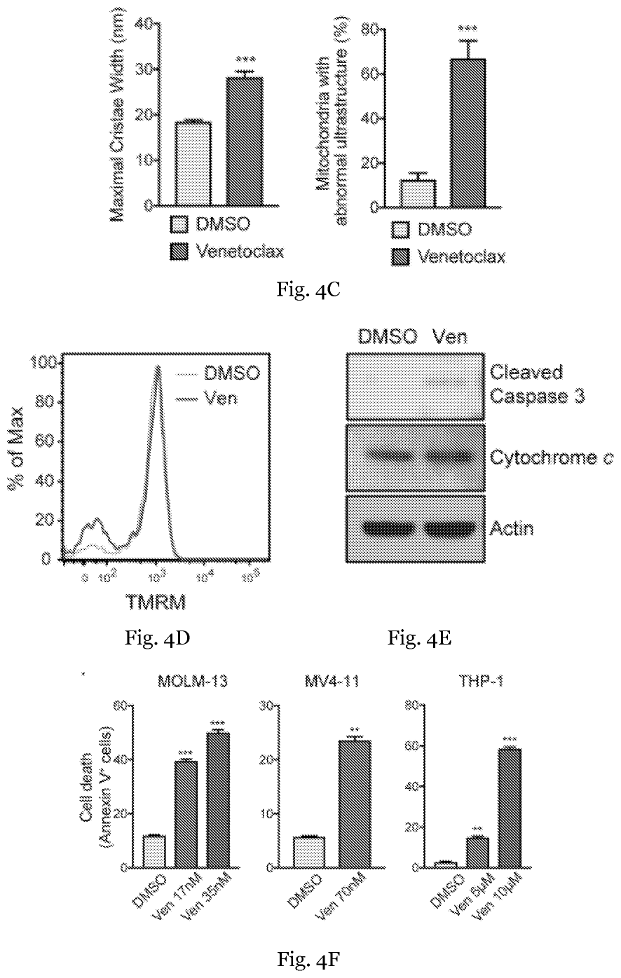

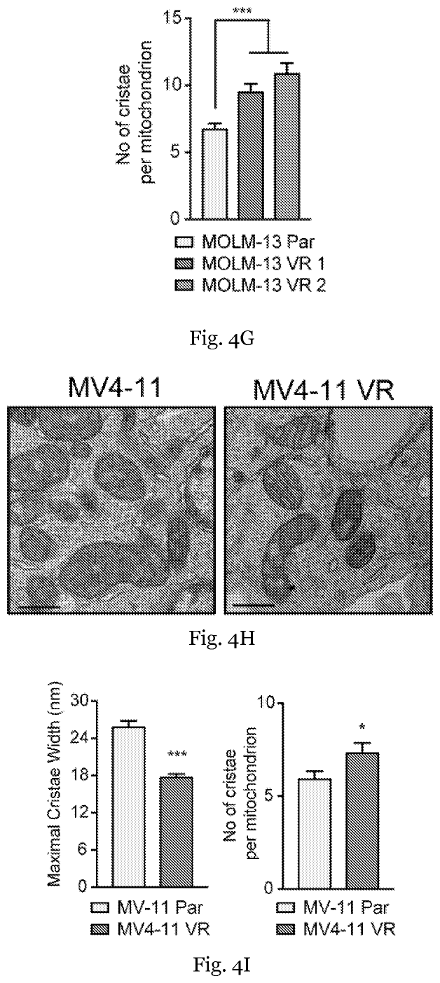

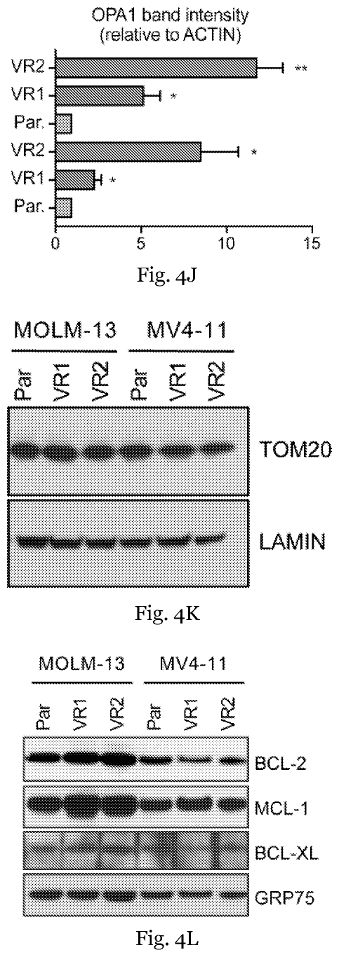

Given that Venetoclax action converges on mitochondria which actively participate in programmed cell death, the screen results led to further investigation regarding how BCL-2 inhibition impacts mitochondrial structure and function in AML. Mitochondria change their shape early during the process of apoptosis; the individual inner membrane lamellae, named cristae, fuse and the opposing faces of the cristae membrane (cristae junctions) open, allowing the redistribution of cytochrome c from the cristae lumen to the intermembrane space (Wasilewski et al., “The Changing Shape of Mitochondrial Apoptosis.” Trends Endocrinol. Metab. 20:287-94 (2009); and Pernas et al., “Mito-Morphosis: Mitochondrial Fusion, Fission, and Cristae Remodeling as Key Mediators of Cellular Function,” Annu. Rev. Physiol. 78:505-31 (2016), which are each hereby incorporated by reference in their entirety). To determine the mitochondrial structure alterations in AML, electron microscopy-based morphometric analysis of treated AML cells (THP-1 and MOLM-13) with Venetoclax was performed. Indeed, mitochondria exhibit abnormal ultrastructure after Venetoclax treatment, characterized by a lower number of cristae and increase of the cristae lumen width (FIGS. 3A-3C, 4A-4C). The normal mitochondrial structure is crucial for the maintenance of the mitochondrial membrane potential, which is the driving force behind ATP production and mitochondrial homeostasis. Early during apoptosis, the mitochondrial membrane potential collapses leading to the complete cytochrome c release from mitochondria to the cytosol (Burke, “Mitochondria, Bioenergetics and Apoptosis in Cancer,” Trends Cancer 3:857-70 (2017), which is hereby incorporated by reference in its entirety). It was therefore predicted that Venetoclax, as an inducer of apoptosis, causes loss of mitochondrial membrane potential in leukemic cells. Indeed, staining of AML cells with the cell-permeant, cationic fluorescent dye, tetramethylrhodamine methyl-ester (TMRM) revealed depolarization of mitochondria after treatment with the BCL-2 inhibitor (FIGS. 3D, 4D). To further investigate how Venetoclax treatment may be causing widening of the cristae, the mitochondrial protein Optic Atrophy 1 (OPA1), which functions as a molecular staple at cristae junctions to prevent cytochrome c mobilization and release (Frezza et al., “OPA1 Controls Apoptotic Cristae Remodeling Independently from Mitochondrial Fusion,” Cell 126:177-89 (2006), which is hereby incorporated by reference in its entirety), was investigated. In physiological conditions, membrane-associated (long) and soluble, cleaved (short) forms of OPA1 coexist to form oligomers and complexes that contribute to cristae maintenance. Upon an intrinsic death stimulus, OPA1 proteolytical cleavage is enhanced allowing the “opening” of the cristae junctions and the cytochrome c redistribution (Burke, “Mitochondria, Bioenergetics and Apoptosis in Cancer,” Trends Cancer 3:857-70 (2017), which is hereby incorporated by reference in its entirety). A decrease in the long (L-OPA1) forms and accumulation of short (S-OPA1) forms of OPA1 in response to Venetoclax treatment was observed, suggesting its proteolysis following BCL-2 inhibition (FIGS. 3E, 3F). Venetoclax treatment also led to caspase activation, as indicated by caspase-3 cleavage (FIG. 4E) and subsequently cell death, as demonstrated using Annexin-V staining (FIG. 4F).

### Example 3—Venetoclax Resistance is Coupled to OPA1 Overexpression and Tighter Mitochondrial Cristae

To further investigate the mechanisms of Venetoclax resistance, four human AML clones highly resistant to the drug were generated by growing the parental MOLM-13 and MV4-11 cells in increasing doses of the drug for over eight weeks. IC₅₀ analysis verified more than 100-fold increase of Venetoclax concentration required for the 50% cell death in the Venetoclax-resistant (VR) cell lines in respect to the original clones (Par.) (FIG. 3G). Driven by the role of BCL-2 inhibition in mitochondrial architecture, the organelle's structure in the Venetoclax-resistant AML cells was closely examined. It was discovered that Venetoclax-resistant AML cells exhibit tighter cristae (approximately 14-15 nm in diameter) and a higher number of cristae per mitochondrion than the parental sensitive clones (FIGS. 3H, 3I, 4G-4I). This phenotype could be explained by the significant induction of OPA1 protein expression in the AML Venetoclax-resistant cell lines (FIGS. 3J, 4J). Notably, this is not simply the result of a general increase of mitochondrial biogenesis, as the mitochondrial protein TOM20 does not display similar expression changes (FIG. 4K). These data suggested that mitochondria change their shape in Venetoclax-resistant AML cells, most likely to antagonize the cell intrinsic apoptotic signaling cascade initiated by BCL-2 inhibition.

Since MCL-1 upregulation has been previously implicated in the development of Venetoclax-resistance (Konopleva et al., “Efficacy and Biological Correlates of Response in a Phase II Study of Venetoclax Monotherapy in Patients with Acute Myelogenous Leukemia,” Cancer Discov. 6:1106-17 (2016), which is hereby incorporated by reference in its entirety), the protein levels of the major BCL-2 family members (MCL-1, BCL-2 and BCL-XL) in the generated Venetoclax-resistant cell lines was examined. A slight increase of MCL-1 and BCL-2 protein levels in MOLM-13-VR cells was observed. Also no increase of BCL-XL protein nor any of the examined BH3 anti-apoptotic proteins in the MV4-11-VR cell lines was found. These results suggest that BCL-2 and MCL-1 upregulation may serve as one of the potential mechanisms of cellular resistance to Venetoclax, however at least in AML, it appears to exist alternative modes of resistance acquisition (FIG. 4L). In agreement with that notion, and based on the generated resistant AML lines, OPA1 upregulation and mitochondrial structural adaptations appear to represent a more universal mechanism of Venetoclax resistance.

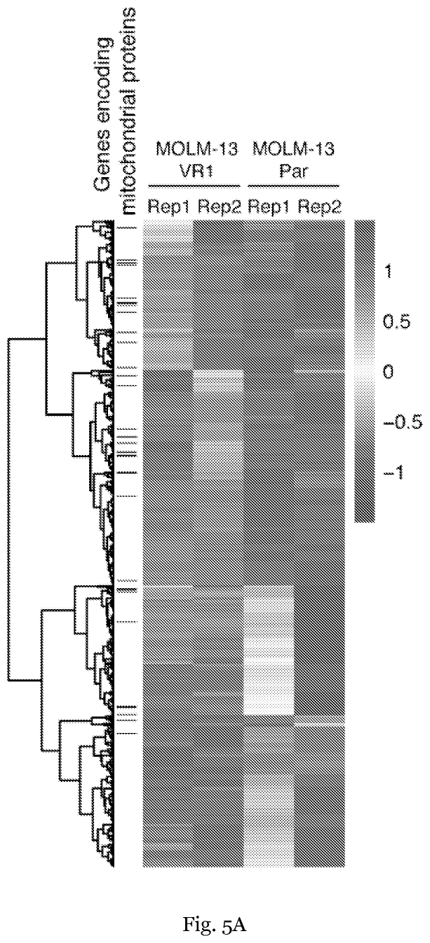

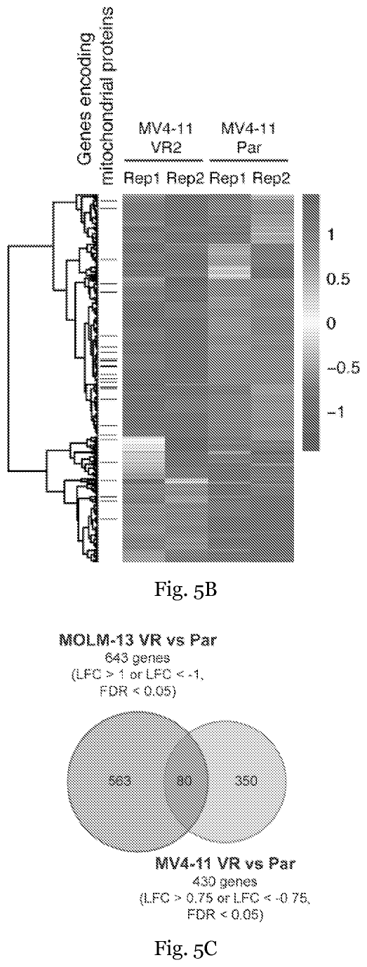

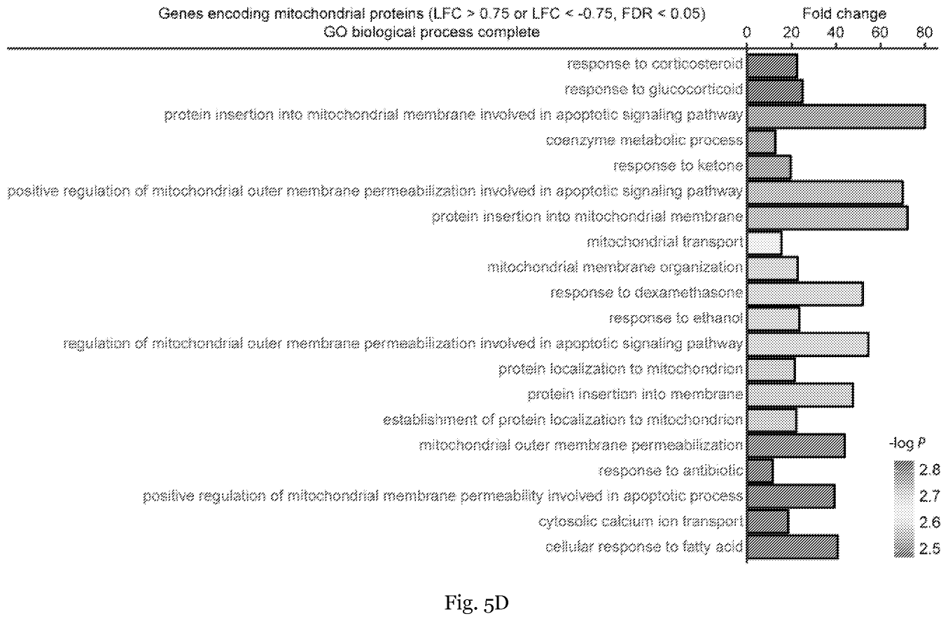

To further explore the molecular mechanisms underpinning Venetoclax resistance, RNA-sequencing (RNA-Seq) was performed. Indeed, the transcriptional landscape of the AML cells changed significantly upon acquisition of resistance to Venetoclax (FIGS. 5A-5B). Interestingly, among the differentially expressed genes, there were many involved in various mitochondrial processes. Such genes (“Genes encoding mitochondrial proteins”, using the MitoMiner dataset; FIGS. 5A-5B) were focused on, and GO analysis was performed to dissect the functional changes in mitochondria upon acquisition of Venetoclax resistance. Strikingly, pathways that regulate mitochondrial membrane organization, potential and depolarization were strongly enriched in both MOLM-13-VR and MV4-11-VR, though strong variances were observed at global transcriptomic level in these cell lines (FIGS. 3K, 5C-5D), which is consistent with the findings that mitochondrial structure adaptation is universally required for gain of Venetoclax resistance in AML cells. Besides, metabolic processes of key cellular components, like amino acids, coenzyme, ATP and nucleotides were also enriched within the top 20 enriched pathways, suggesting the metabolic adaptation is likewise important for Venetoclax resistance. Finally, enhanced interferon responses in MOLM-13-VR cells were observed, which might be a result of mtDNA release to the cytosol as a consequence of incomplete apoptosis and partial mitochondrial dysfunction during the persistent Venetoclax treatment (West et al., “Mitochondrial DNA Stress Primes the Antiviral Innate Immune Response,” Nature 520:553-7 (2015), which is hereby incorporated by reference in its entirety) (FIGS. 3K, 5D). Overall, these data suggested that AML blasts require transcriptional and post-translational mitochondrial adaptation to acquire resistance to Venetoclax.

### Example 4—Targeting the Mitochondrial Protein CLPB Overcomes Venetoclax Resistance in AML

Next it was determined whether there is a correlation between genes differentially expressed in the resistant cell lines and genes-hits in the CRISPR screen. Initially, the downregulated genes in MOLM-13-VR cells compared to MOLM-13-Par cells, whose ablation confers resistance to Venetoclax (positively selected genes) were focused on. The validated genes TP53, BAX, PMAIP1, as well as the pro-apoptotic genes BAD and BCL2L11 (also known as BIM), and SLC27A2, a gene involved in fatty acid metabolism were identified. Moreover, the upregulated genes in MOLM-13-VR cells compared to MOLM-13-Par cells, whose ablation sensitizes cells to the drug (negatively selected genes) were also focused on. The mitochondrial proteins CLPB and SLC25A1, as well as anti-apoptotic genes like MCL-1 and HAX1 were focused on. ATPAF1 and MSTO1 whose products are involved in the maintenance of mitochondrial structure and functions were also identified (FIG. 3L).

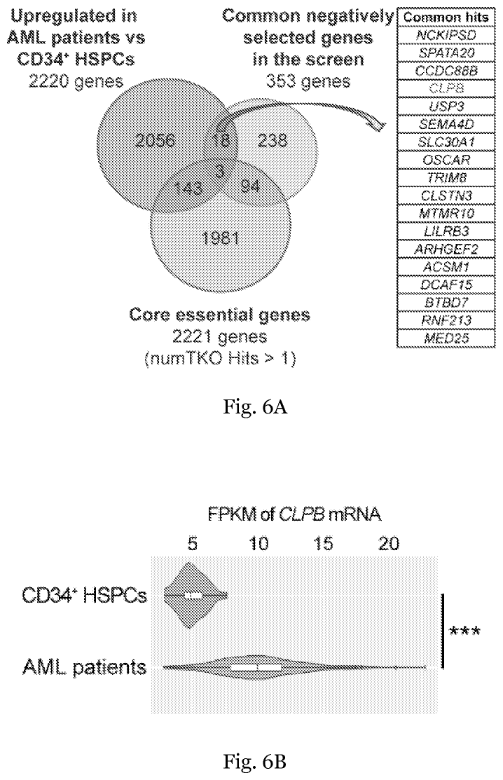

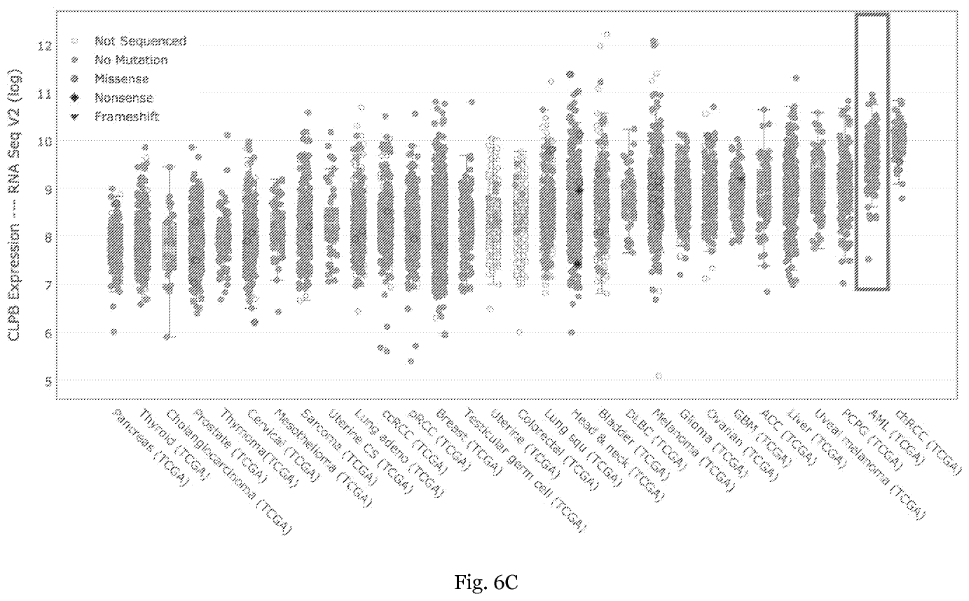

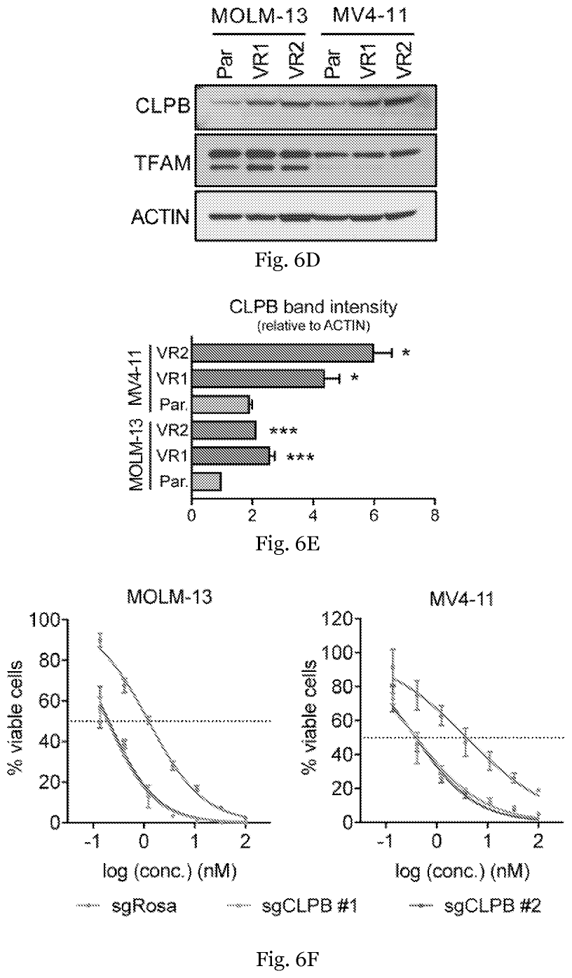

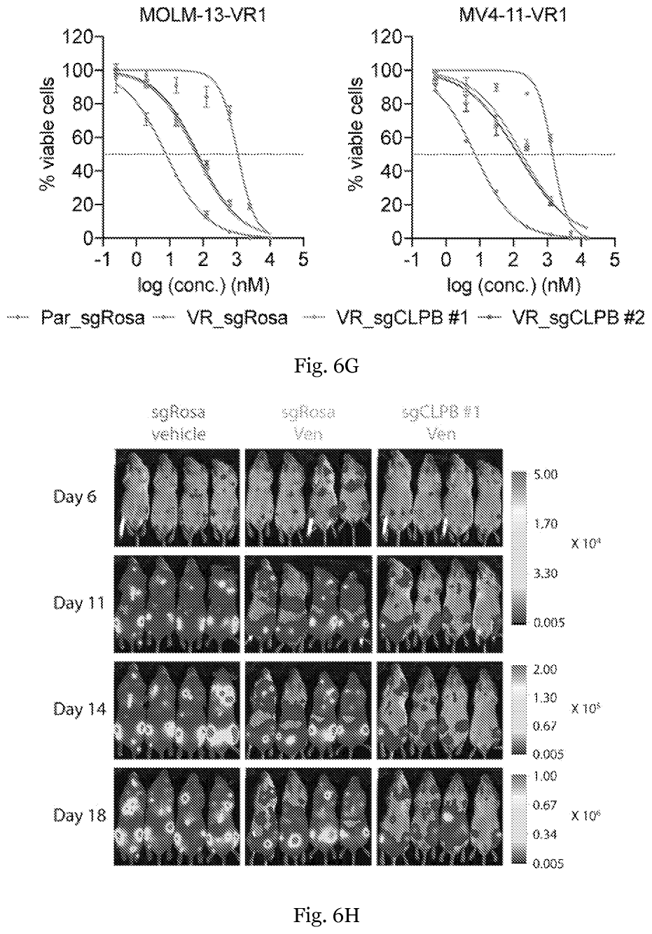

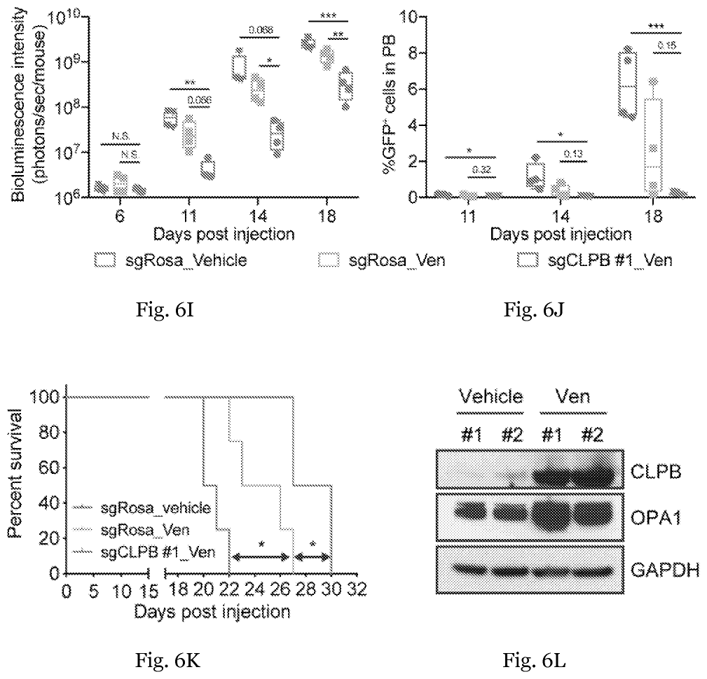

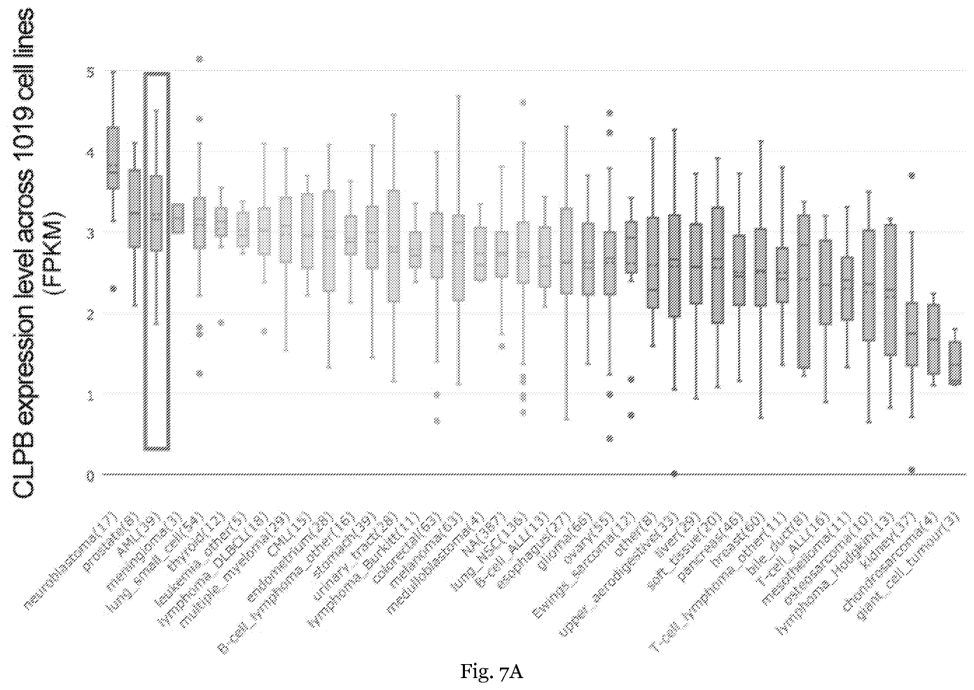

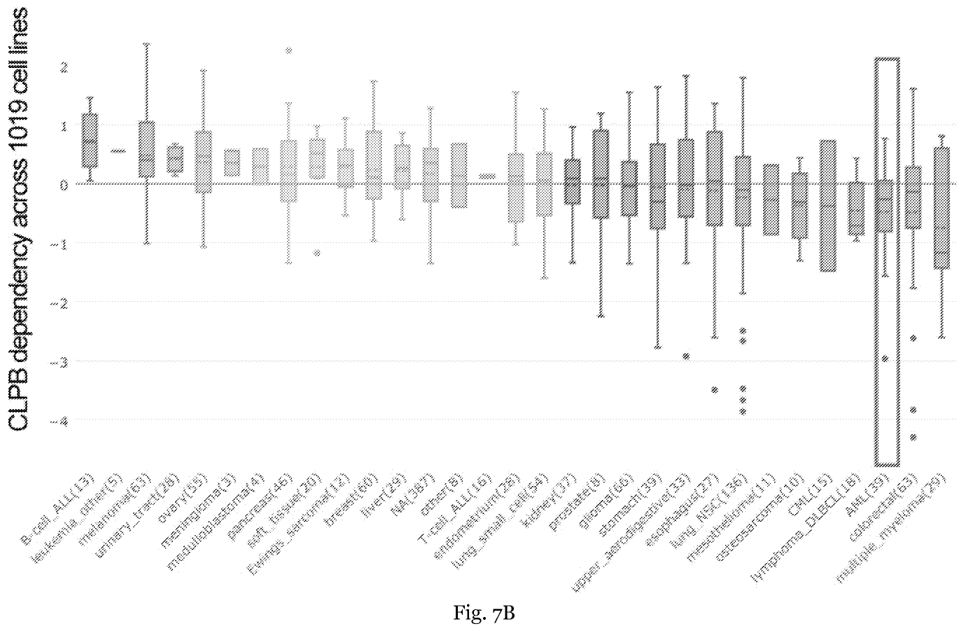

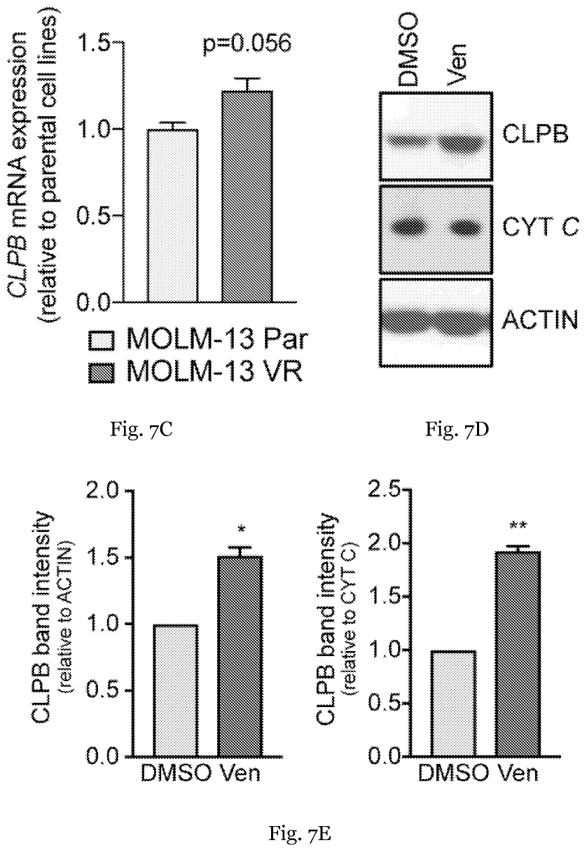

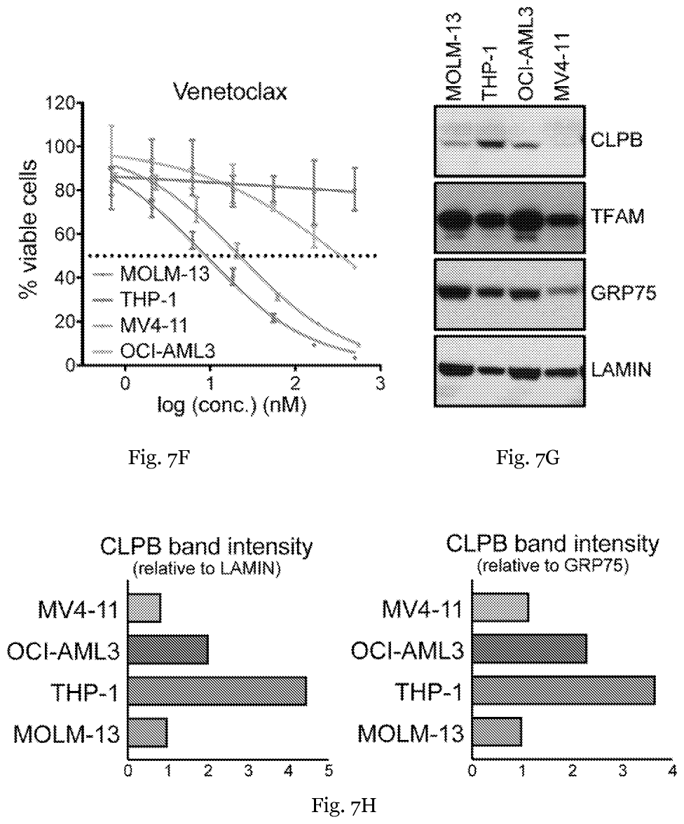

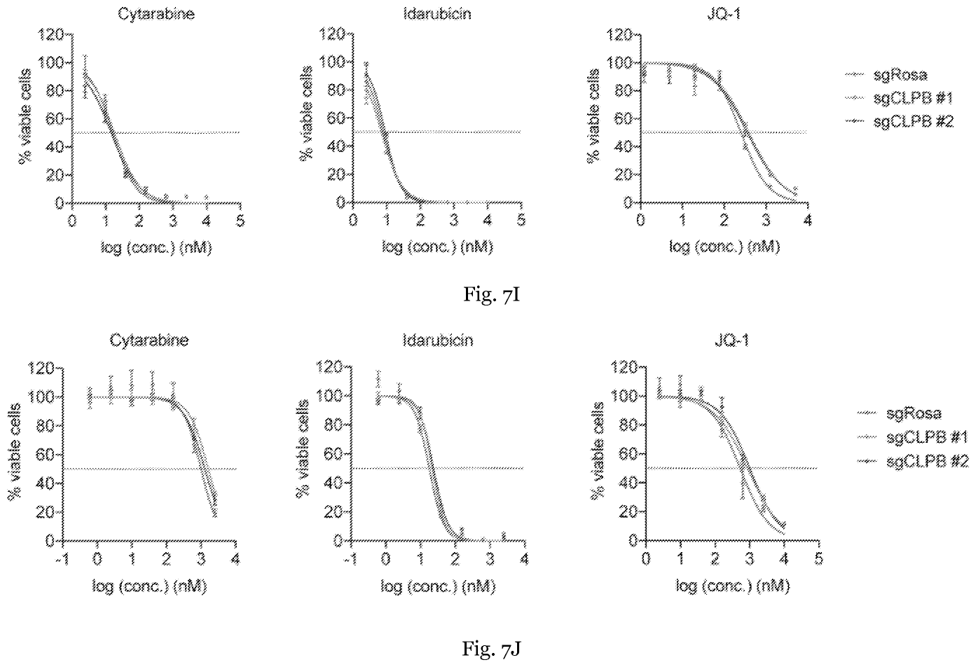

Driven by these findings, it was hypothesized that targeting proteins regulating mitochondrial cristae maintenance and function might open up potential synthetic lethal vulnerabilities for Venetoclax treatment. To further refine the candidate list from the screen, 97 core cellular fitness genes (Hart et al., “High-Resolution CRISPR Screens Reveal Fitness Genes and Genotype-Specific Cancer Liabilities,” Cell 163:1515-26 (2015), which is hereby incorporated by reference in its entirety), genes generally required for all cell types and therefore may not serve as ideal future therapeutic targets, were excluded. From the remaining 256 genes, genes that displayed higher mRNA expression levels in AML patient samples compared to healthy primary CD34+ hematopoietic stem and progenitor cells (HSPCs) according to The Cancer Genome Atlas (TCGA) were focused on. After applying these criteria, 18 candidates were identified (FIG. 6A). One of the top-scoring candidates was CLPB, that encodes a mitochondrial AAA+ ATPase chaperonin. The expression studies revealed that CLPB gene expression is significantly higher in AML patient samples when compared to normal CD34+ HSPCs (FIG. 6B). Moreover, CLPB median expression in AML was the second highest among all other cancer subtypes in the TCGA dataset and the third highest among all cancer cell lines in the Cancer Cell Line Encyclopedia (CCLE) database (FIGS. 6C, 7A).

As a further suggestion of a role in this process, higher levels of CLPB protein in Venetoclax-resistant human AML cell lines when compared to the parental sensitive clones were observed (FIGS. 6D, 6E). Based on the RNA-Seq data (FIG. 7C), CLPB transcripts are only slightly upregulated in Venetoclax-resistant cells, indicating that this overexpression is post-translational. Importantly, the protein levels of the mitochondrial transcription factor A (TFAM) were unchanged, indicating that the total mitochondrial mass is not altered. Furthermore, when AML cells were treated with Venetoclax, a significant increase in the CLPB protein was observed, indicating that cells that survive Venetoclax treatment have higher CLPB levels (FIGS. 7D-7E). In addition, the CLPB protein levels in 4 different AML cell lines (MOLM-13, MV4-11, OCI-AML3 and THP-1) were quantified, and it was found that cell lines with high CLPB protein levels were more resistant to Venetoclax than those with low CLPB levels (FIGS. 7F-7H); however, the small statistical sample of cell lines analyzed precludes suggesting a definite predictive role of CLPB protein levels to Venetoclax sensitivity.

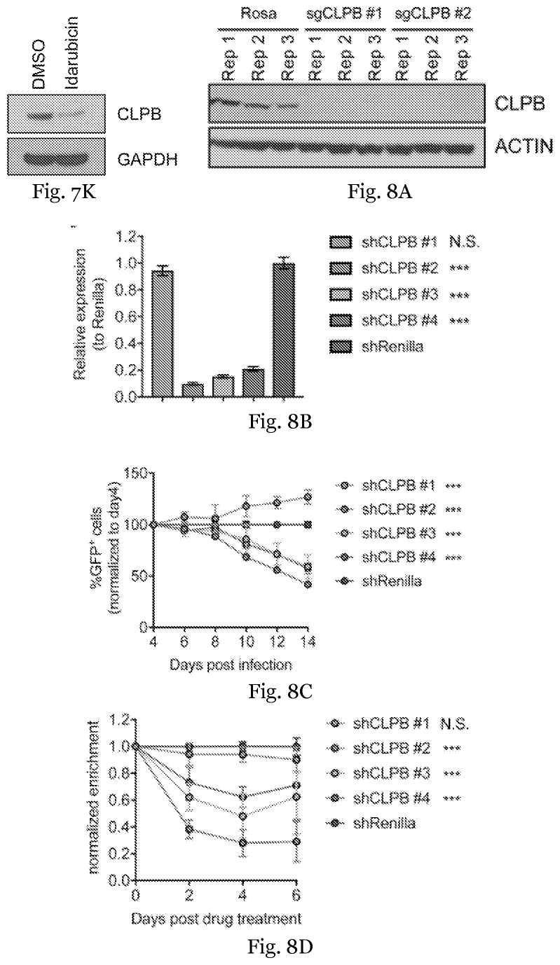

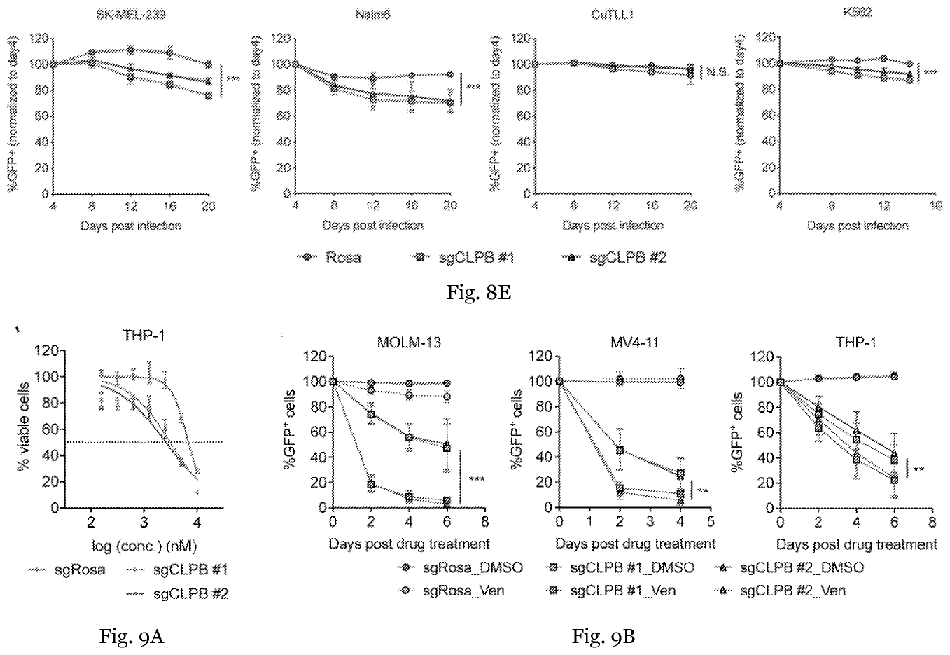

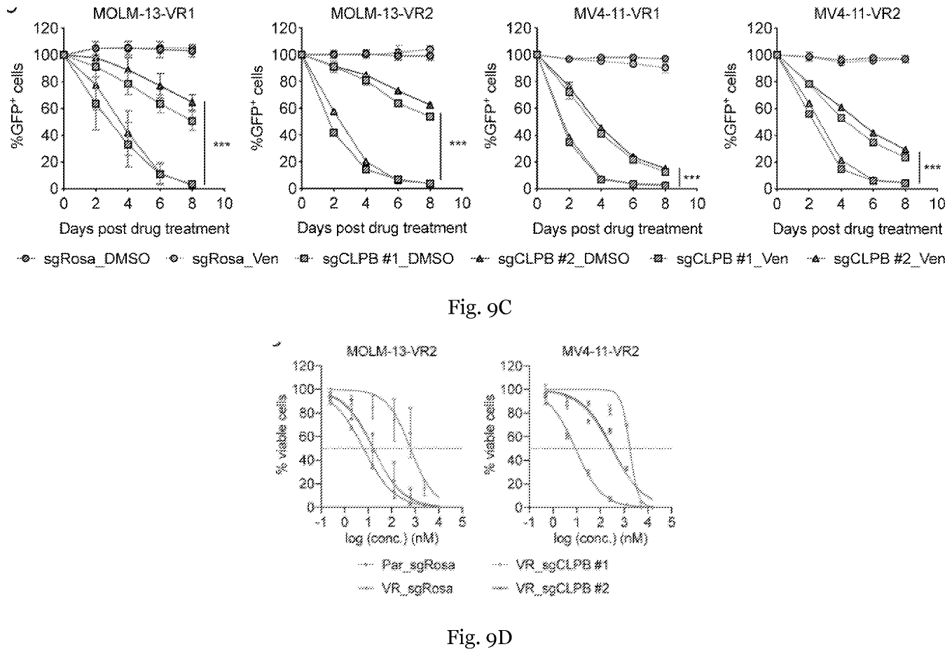

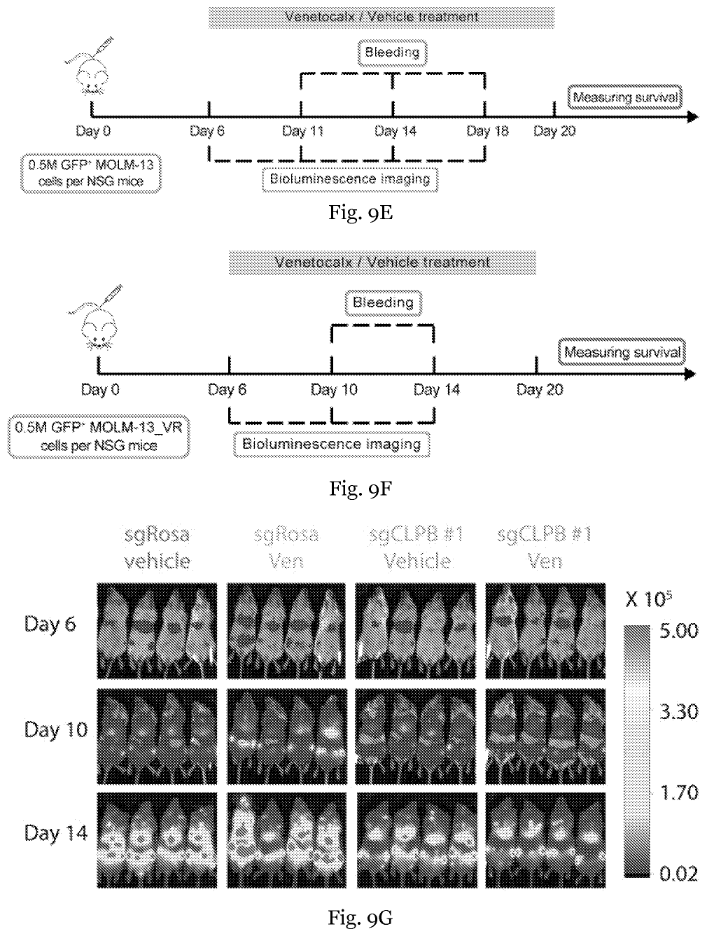

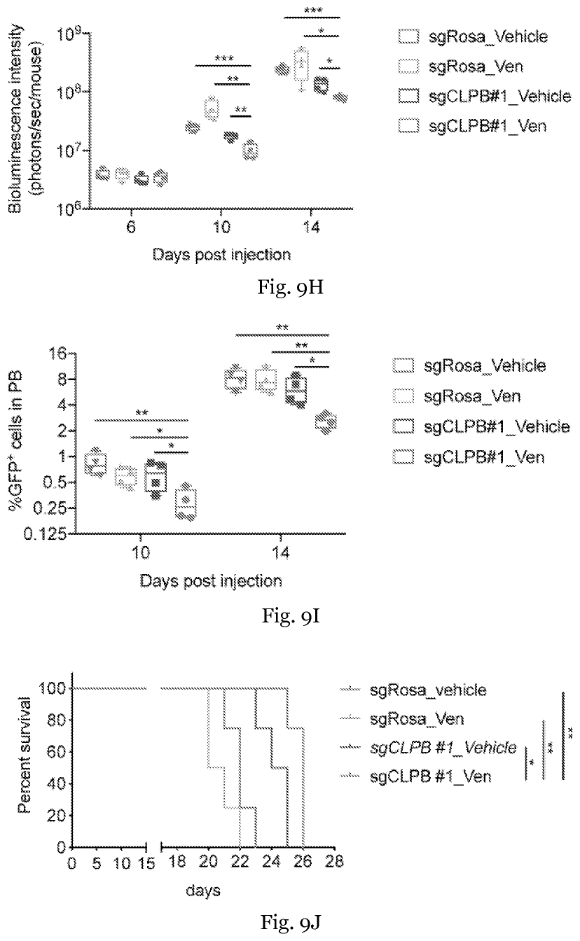

To further evaluate the importance of CLPB function across different human AML cells, CLPB was depleted in multiple AML cell lines using CLPB-targeting sgRNAs (FIG. 8A), and the Venetoclax IC₅₀ was subsequently measured. CLPB depletion significantly reduced the IC₅₀ of Venetoclax in all the cell lines tested (FIGS. 6F, 9A). Additionally, CLPB-deficient AML cells were negatively selected upon Venetoclax addition in a much faster pace than the DMSO control groups in a competition-based viability assay (FIG. 9B), confirming that CLPB ablation can sensitize AML cells to Venetoclax treatment. Considering that CLPB is stabilized in Venetoclax-resistant AML cells (FIGS. 6D, 6E), the sensitization effect of CLPB depletion in the Venetoclax-resistant cells were sought to be validated. Similar to the results observed in the parental clones (FIG. 6F), ablation of CLPB significantly re-sensitized VR cells to the drug treatment (FIGS. 6G, 9C-9D). The synthetic lethal relationship between CLPB ablation and Venetoclax is specific to Venetoclax-mechanisms of action, since no synergistic effect was detected in AML cells treated with Cytarabine, Idarubicin or JQ-1 treatment (FIGS. 7I-7J). Moreover, in contrast to Venetoclax, treatment of MOLM-13 with Idarubicin did not lead to increase of CLPB protein levels in the surviving cells (FIG. 7K). Finally, to avoid potential side-effects of CRISPR-mediated gene deletion, silencing of CLPB using four distinct shRNAs with different efficacies further validated the dose dependent competitive disadvantage of CLPB loss in MOLM-13 treated with Venetoclax (FIGS. 8B-8D). Altogether, these findings revealed a functional role of mitochondrial CLPB in the response of AML cells to Venetoclax and suggested that targeting CLPB can overcome Venetoclax resistance in AML.

Finally, the synergistic effects of CLPB deletion with Venetoclax were examined in vivo by using pre-clinical animal models. Specifically, NOD SCID gamma (NSG) mice were transplanted with MOLM-13-Par cells or MOLM-13-VR cells transduced with sgRosa or sgCLPB. After transplantation, the mice were treated with Venetoclax or vehicle from day 6 to day 20. Bioluminescence imaging and examination of peripheral blood circulating leukemic cells (GFP+) indicated that CLPB deletion synergizes efficiently with Venetoclax in both Venetoclax-sensitive and resistant xenografts (FIGS. 6H-6K, 9E-9J). In the course of these in vivo experiments, it was determined that mice bearing wild-type tumors and being treated with Venetoclax exhibit significantly elevated CLPB and OPA1 protein levels at late stages of tumor progression compared to the vehicle-treated mice (FIG. 6L), suggesting once more that mitochondrial structure adaptation occurs also in vivo as a mechanism of resistance to the drug.

### Example 5—CLPB Ablation Impairs Mitochondrial Structure in AML Cells Rendering them More Susceptible to Apoptosis

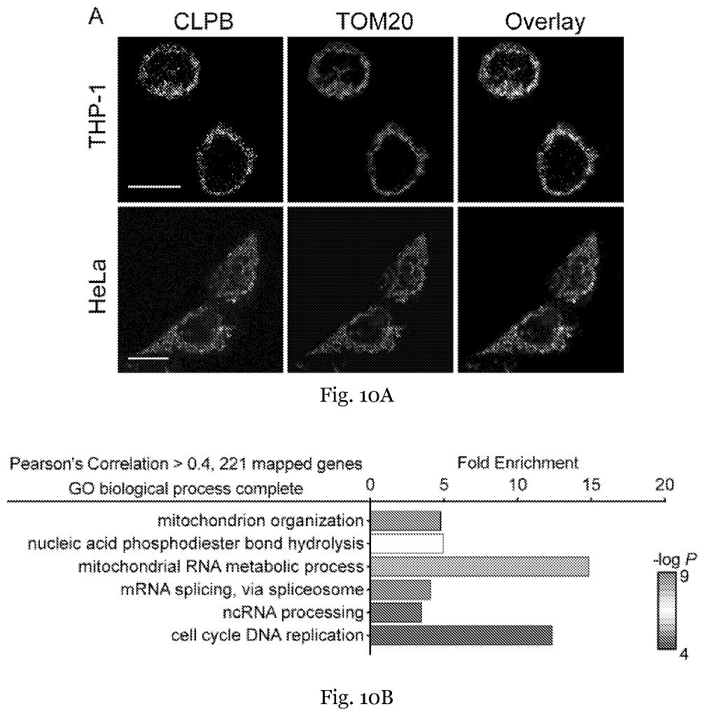

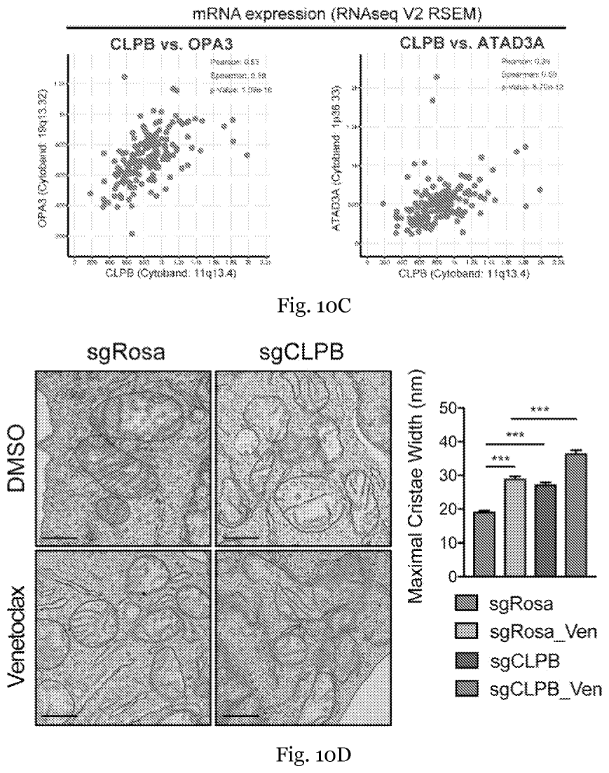

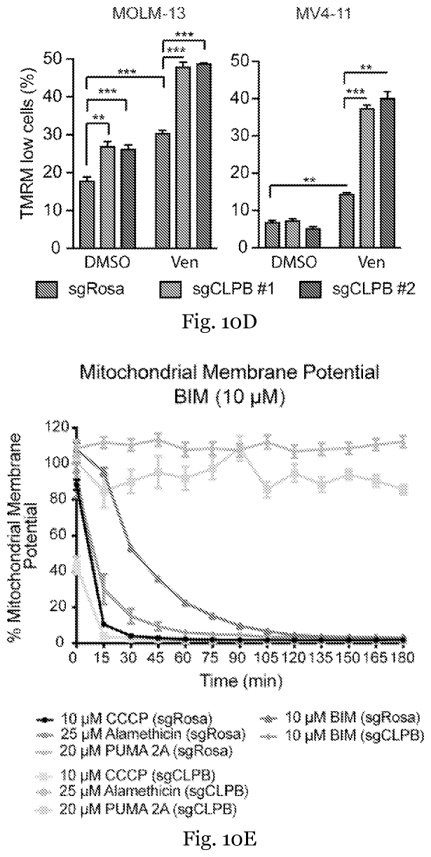

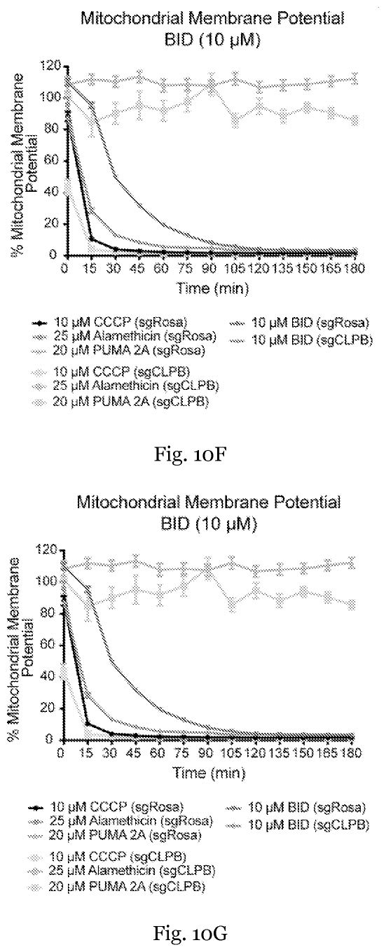

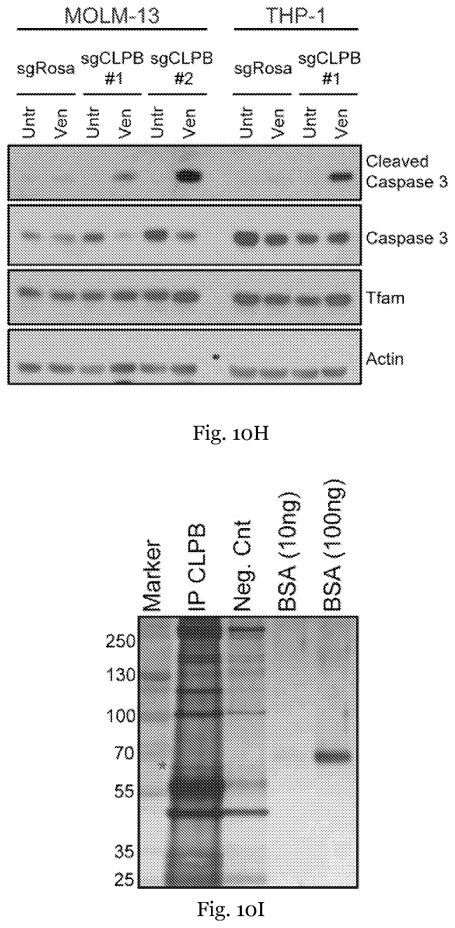

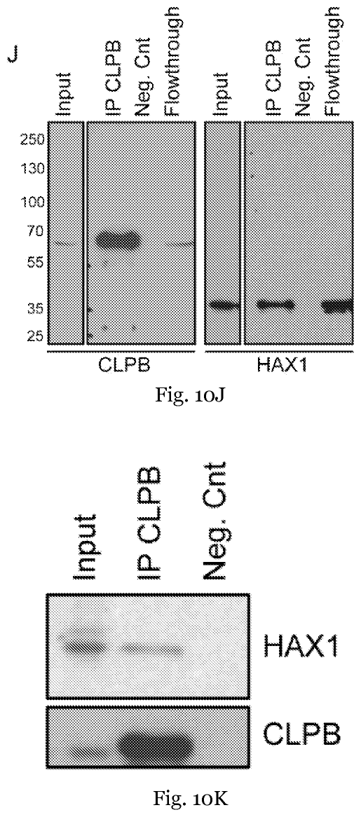

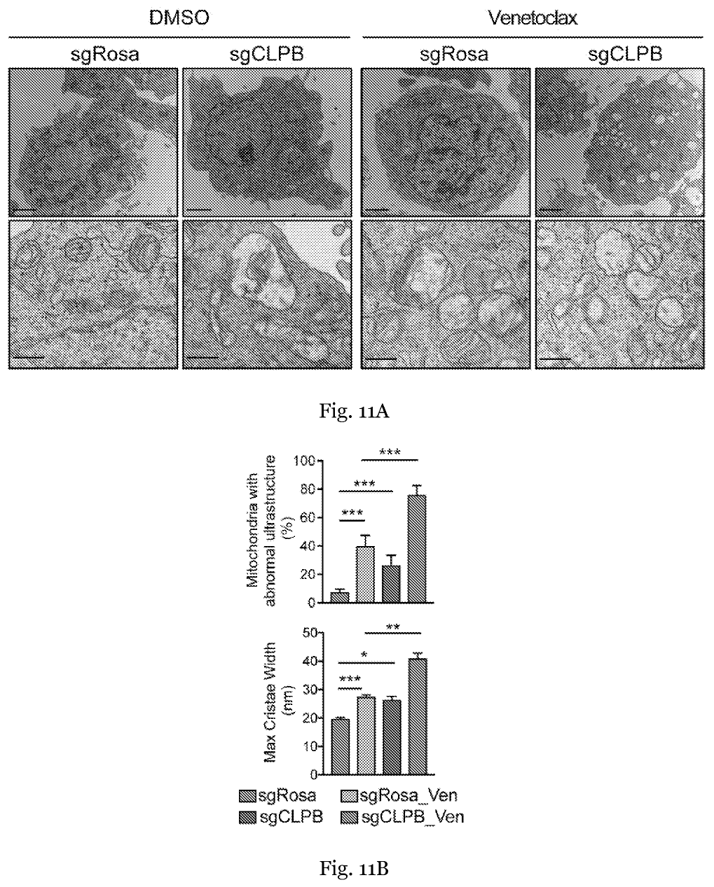

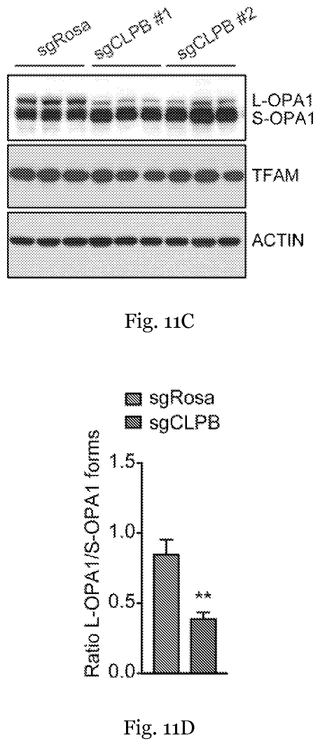

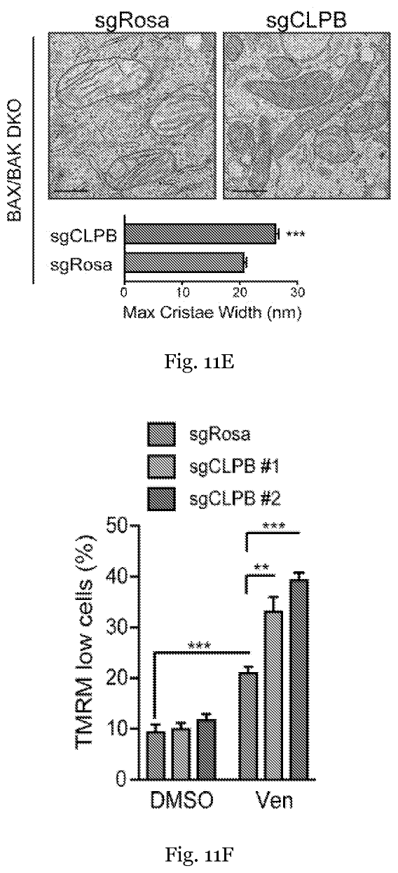

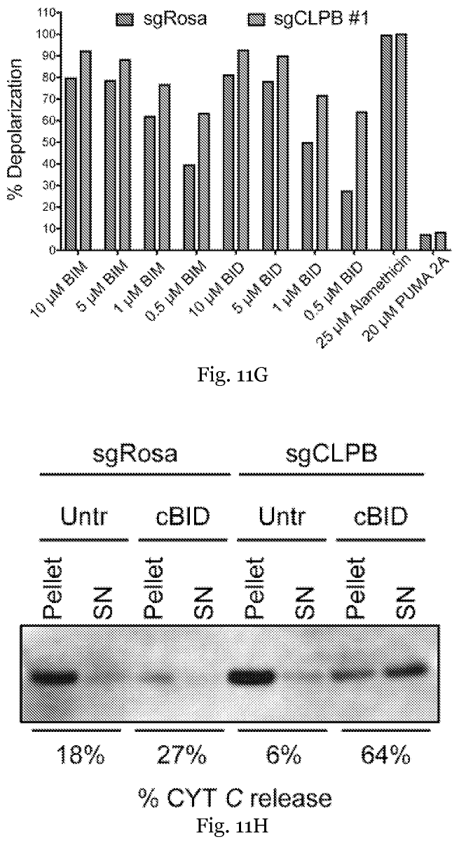

Next, it was investigated how CLPB loss promotes Venetoclax-induced programmed cell death in AML cells. First, to confirm the subcellular localization of CLPB, confocal imaging was performed, which indicated that endogenous CLPB co-localizes with the mitochondrial marker TOM20 in THP-1 and HeLa cells (FIG. 10A). Notably, CLPB expression in patients with AML correlates with the transcription of genes whose products participate in the control of mitochondrial organization, such as OPA3 and ATAD3A (FIGS. 10B, 10C), suggesting a similar function of CLPB in leukemic cells. To test the hypothesis that CLPB participates in the organization of mitochondrial ultrastructure, electron microscopy was performed in CLPB-knockout AML cells. Mitochondria lacking CLPB exhibited an aberrant structure, characterized by wider cristae lumen (maximal cristae width—from a mean of 18 nm in the controls to 27 nm in the knockout). As expected, the mitochondrial structure abnormalities were exacerbated when the cells were treated overnight with Venetoclax and they were more significant in mitochondria lacking CLPB compared to wild-type (FIGS. 11A-11B, 10D). The structural perturbations of CLPB-ablated mitochondria were accompanied by a prominent accumulation of short OPA1 forms, indicating excessive OPA1 processing, as demonstrated by western blotting (FIGS. 11C, 11D). These phenomena were observed also in cells deficient for BAX and BAK, which do not respond to cell death stimuli, suggesting that the mitochondrial morphological defects are not a simple result of undergoing apoptosis (FIG. 11E). In addition, TMRM staining after Venetoclax treatment indicated an elevated percentage of cells with depolarized mitochondria in AML cells lacking CLPB, suggesting that CLPB-knockout cells are more prone to Venetoclax-induced mitochondrial depolarization (FIGS. 11F, 10E). To expand on these results, a “BH3 profiling” was performed, in which permeabilized sgCLPB and sgRosa-expressing AML cells were treated with increasing doses of BH3 peptides and mitochondrial membrane potential was monitored over time using the JC-1 dye. As expected, CLPB-deficient AML cells undergo faster depolarization and are more primed for cell death than wild-type counterparts upon treatment with the BH3-only “activators”, BIM and BID (FIGS. 11G, 10F-10G). Given the wider cristae and the higher sensitivity to mitochondrial membrane potential loss upon apoptosis stimulation, it was speculated that CLPB-deficient mitochondria would readily release cytochrome c following a death stimulus. To address this hypothesis, functional mitochondria were isolated from wild-type and CLPB-knockout THP-1 cells, stimulated with the recombinant proapoptotic caspase-8 cleaved BID (cBID) and centrifuged to retrieve both the pellet (mitochondria) and supernatant (released factors) fractions. Western blot analysis indicated that CLPB-deficient mitochondria are more sensitive to cytochrome c release upon cBID stimulation compared to the wild-type organelles (FIG. 11H). All the above suggest an increased responsiveness of CLPB-ablated AML cells to programmed cell death. Indeed, immunoblotting against pro- and cleaved caspase-3 verified a prevalent caspase-3 activation upon BCL-2 inhibition in AML cells lacking CLPB relative to the wild-type (FIG. 10H). Moreover, Annexin-V staining in puromycin-selected AML cells transduced with sgRNAs targeting CLPB confirmed hypersensitivity of these cells to Venetoclax compared to the negative controls (FIG. 11I). Taken together, these data suggest that CLPB is an important protein for the maintenance of physiological mitochondrial cristae, as well as the control of cytochrome c release and ultimately the execution of cell death.

### Example 6—CLPB Interacts with the Anti-Apoptotic Mitochondrial Proteins HAX1 and OPA1

To investigate how CLPB participates in the preservation of the proper mitochondrial ultrastructure and the regulation of apoptosis, it was sought to identify the interacting partners of this chaperonin in human AML cells. Immunoprecipitation (IP) of the endogenous human CLPB followed by mass spectrometry (MS) in whole THP-1 cell lysates was performed (FIG. 10I). This analysis uncovered 64 mitochondrial proteins found exclusively in the CLPB pull-down and not in the IgG sample (negative control), indicating their potential association with CLPB. Among the top enriched CLPB interactors, two proteins involved in the regulation of mitochondrial morphology and apoptosis, HAX1 and OPA1 (Frezza et al., “OPA1 Controls Apoptotic Cristae Remodeling Independently from Mitochondrial Fusion,” Cell 126:177-89 (2006); and Yan et al., “HAX-1 Inhibits Apoptosis in Prostate Cancer Through the Suppression of Caspase-9 Activation,” Oncol. Rep. 34:2776-81 (2015), which are each hereby incorporated by reference in their entirety), were identified, ranked No. 1 and 3, respectively (Table 1). Western blotting of the CLPB immunoprecipitation in THP-1 lysates and mouse liver mitochondria confirmed the physical interaction of CLPB with HAX1 and OPA1 (FIGS. 11J, 10J, 10K). Altogether these data suggest that CLPB regulates mitochondrial cristae morphology and apoptosis in AML cells, via its specific interactions with OPA1 and HAX1.

TABLE 1
CLPB interacting mitochondrial proteins

| Accession | Score | Coverage | # Peptides | # PSM | # Aas | Entry |
|---|---|---|---|---|---|---|
| O00165-5 | 47.37 | 67.1 | 10 | 17 | 231 | HAX1_HUMAN |
| Q00325-2 | 34.82 | 16.62 | 6 | 12 | 361 | MPCP_HUMAN |
| O60313 | 29.55 | 10.83 | 6 | 9 | 960 | OPA1_HUMAN |
| P28288-2 | 28.01 | 13.11 | 7 | 8 | 549 | ABCD3_HUMAN |
| P04181 | 20.42 | 27.56 | 8 | 8 | 439 | OAT_HUMAN |
| Q86UT6-2 | 12.21 | 6.41 | 6 | 8 | 921 | NLRX1_HUMAN |
| O75165 | 16.09 | 2.81 | 5 | 5 | 2243 | DJC13_HUMAN |
| A0A0G2JQM1 | 12.22 | 9.78 | 4 | 5 | 634 | A0A0G2JQM1_HUMAN |
| Q9UJS0 | 15.67 | 11.11 | 4 | 5 | 675 | CMC2_HUMAN |
| Q9P035 | 15.51 | 15.47 | 4 | 5 | 362 | HACD3_HUMAN |
| P10074 | 14.75 | 7.27 | 3 | 5 | 688 | ZBT48_HUMAN |
| A0A087WXC5 | 13.14 | 15.49 | 4 | 4 | 355 | A0A087WXC5_HUMAN |
| Q9UBX3 | 10.53 | 18.82 | 4 | 4 | 287 | DIC_HUMAN |
| O75746-2 | 6.71 | 6.13 | 4 | 4 | 571 | CMC1_HUMAN |
| Q5K4L6 | 12.64 | 7.12 | 2 | 4 | 730 | S27A3_HUMAN |
| P11166 | 12.31 | 5.49 | 2 | 4 | 492 | GTR1_HUMAN |
| Q9NS69 | 9.14 | 25.35 | 2 | 4 | 142 | TOM22_HUMAN |
| O43847 | 8.02 | 2.78 | 2 | 4 | 1150 | NRDC_HUMAN |
| Q8N163 | 13.78 | 8.99 | 4 | 4 | 923 | CCAR2_HUMAN |
| C9IZ01 | 11.79 | 10.83 | 4 | 4 | 591 | C9IZ01_HUMAN |
| O14727-3 | 8.58 | 3.27 | 3 | 3 | 1194 | APAF_HUMAN |
| K7EQ48 | 5.54 | 13.08 | 3 | 3 | 474 | K7EQ48_HUMAN |
| Q14738-3 | 8.33 | 8.27 | 3 | 3 | 496 | 2A5D_HUMAN |
| H0YHS6 | 10.53 | 15.12 | 3 | 3 | 291 | H0YHS6_HUMAN |
| H3BMM5 | 10.84 | 11.9 | 3 | 3 | 538 | H3BMM5_HUMAN |
| P56134-4 | 8.55 | 48.98 | 2 | 3 | 49 | ATPK_HUMAN |
| O60884 | 10.76 | 15.05 | 2 | 3 | 412 | DNJA2_HUMAN |
| Q96EY1-3 | 7.29 | 10 | 2 | 3 | 300 | DNJA3_HUMAN |
| M0R2K9 | 11.84 | 17.51 | 2 | 3 | 257 | M0R2K9_HUMAN |
| E5RI56 | 10.37 | 31.87 | 2 | 3 | 91 | E5RI56_HUMAN |
| H3BQZ7 | 5.73 | 4.83 | 3 | 3 | 746 | H3BQZ7_HUMAN |
| Q15031 | 6.18 | 3.1 | 3 | 3 | 903 | SYLM_HUMAN |
| F5GY32 | 9.29 | 14.38 | 2 | 3 | 153 | F5GY32_HUMAN |
| B4DGL8 | 7.84 | 6.98 | 2 | 2 | 702 | B4DGL8_HUMAN |
| H7C5Q2 | 6.26 | 10.98 | 2 | 2 | 264 | H7C5Q2_HUMAN |
| E5RK69 | 8.91 | 6.96 | 2 | 2 | 460 | E5RK69_HUMAN |
| Q93084-4 | 5.91 | 2 | 2 | 2 | 998 | AT2A3_HUMAN |
| G5E9Z2 | 4.09 | 10.57 | 2 | 2 | 369 | G5E9Z2_HUMAN |
| Q7L576 | 5.38 | 1.68 | 2 | 2 | 1253 | CYFP1_HUMAN |
| O94819 | 6.15 | 4.17 | 2 | 2 | 623 | KBTBB_HUMAN |
| Q9HCC0-2 | 4.93 | 4.19 | 2 | 2 | 525 | MCCB_HUMAN |
| Q5T454 | 8.12 | 12.25 | 2 | 2 | 302 | Q5T454_HUMAN |
| E9PRK3 | 4.61 | 13.61 | 2 | 2 | 169 | E9PRK3_HUMAN |
| C9J4M6 | 6.24 | 2.46 | 2 | 2 | 1099 | C9J4M6_HUMAN |
| P48651-3 | 5.83 | 8.97 | 2 | 2 | 301 | PTSS1_HUMAN |
| Q9Y512 | 3.97 | 3.2 | 2 | 2 | 469 | SAM50_HUMAN |
| G3V2Y4 | 2.28 | 7.69 | 2 | 2 | 234 | G3V2Y4_HUMAN |
| Q15477 | 5.81 | 2.09 | 2 | 2 | 1246 | SKIV2_HUMAN |
| P53007 | 7.02 | 7.72 | 2 | 2 | 311 | TXTP_HUMAN |
| F6S7Q7 | 5.16 | 6.55 | 2 | 2 | 443 | F6S7Q7_HUMAN |
| Q9UHD2 | 6.08 | 5.62 | 2 | 2 | 729 | TBK1_HUMAN |
| Q9NWX6 | 6.44 | 11.41 | 2 | 2 | 298 | THG1_HUMAN |
| Q86WV6 | 5.49 | 6.6 | 2 | 2 | 379 | STING_HUMAN |
| P43897 | 6.29 | 13.54 | 2 | 2 | 325 | EFTS_HUMAN |
| Q709C8-4 | 4.03 | 0.5 | 2 | 2 | 3585 | VP13C_HUMAN |
| Q7L0Y3 | 4.48 | 4.22 | 2 | 2 | 403 | MRRP1_HUMAN |
| J3QR71 | 6.87 | 19.62 | 2 | 2 | 158 | J3QR71_HUMAN |
| Q13057 | 5.77 | 4.96 | 2 | 2 | 564 | COASY_HUMAN |
| Q7Z478 | 8.45 | 2.34 | 2 | 2 | 1369 | DHX29_HUMAN |
| A0A087WUQ6 | 5.22 | 14.85 | 2 | 2 | 202 | A0A087WUQ6_HUMAN |
| Q99714 | 3.5 | 13.03 | 2 | 2 | 261 | HCD2_HUMAN |
| C9J4N6 | 4.47 | 14.01 | 2 | 2 | 157 | C9J4N6_HUMAN |
| Q4G0N4-3 | 4.25 | 6.45 | 2 | 2 | 279 | NAKD2_HUMAN |
| D6RHU2 | 5.6 | 15.08 | 2 | 2 | 199 | D6RHU2_HUMAN |

### Example 7—CLPB Loss Suppresses AML Cell Growth In Vitro and In Vivo

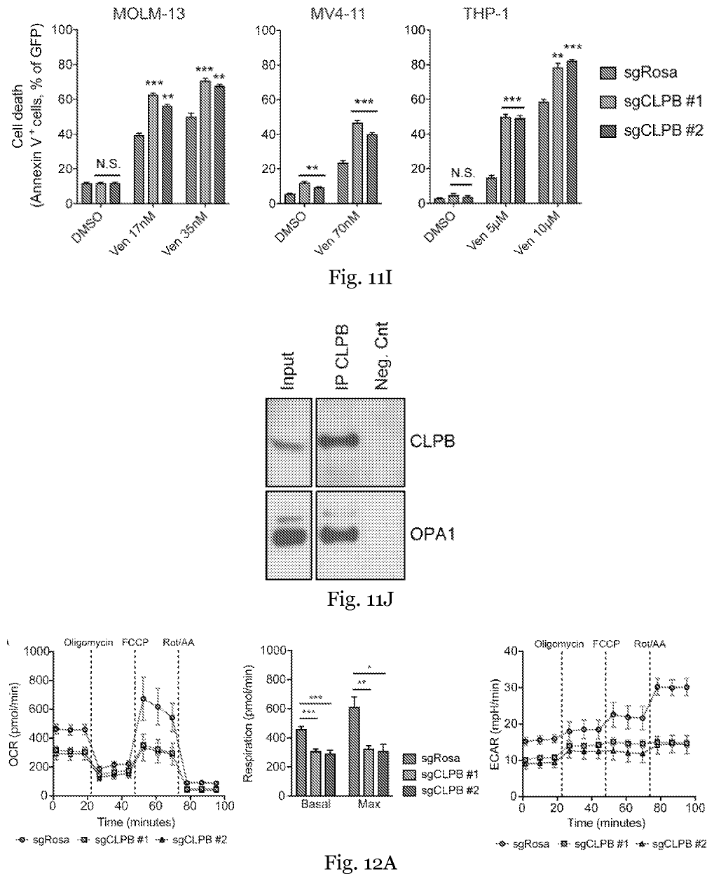

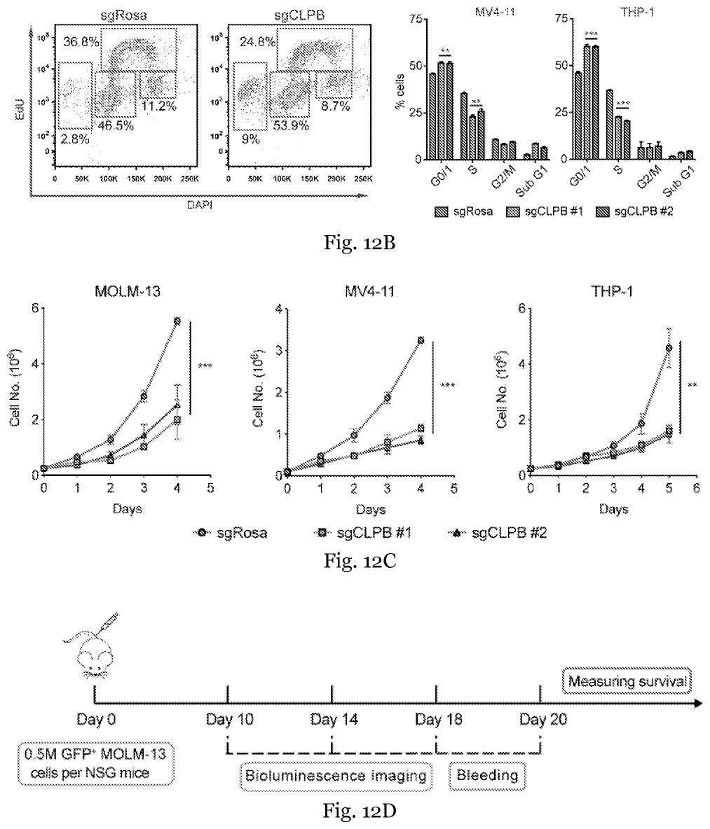

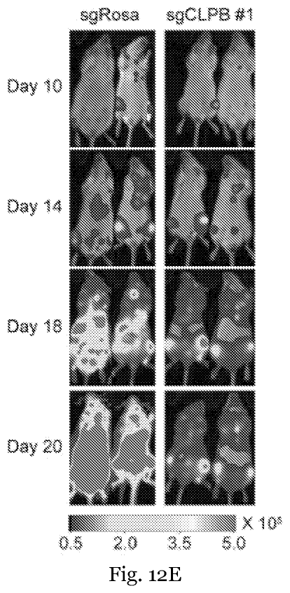

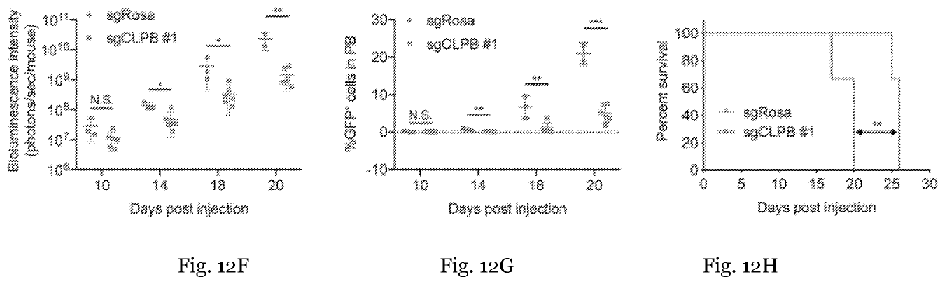

Given the mitochondrial structure defects, it was hypothesized that CLPB ablation directly affects mitochondrial functions. Indeed, using the live cell metabolic assay platform Seahorse XF, impaired basal and maximal mitochondrial respiration was shown, as well as defective glycolysis in CLPB deleted AML, suggesting a more quiescent metabolic state compared to the highly energetic wild-type AML cells (FIG. 12A). It was then asked whether such bioenergetic defects have an impact on cell proliferation. EdU incorporation assays suggested a suppression of cell replication in AML cells deficient of CLPB (FIG. 12B). These cell growth defects measuring cell doublings were verified across time (FIG. 12C). More importantly, suppression of cell growth was not seen or was less pronounced in a number of non-AML cancer cell lines, including T-ALL, B-ALL, CML and melanoma (FIG. 8E), highlighting once more the specificity of CLPB dependency in AML cells (FIG. 7B).

Finally, the in vivo significance of CLPB in AML progression was investigated using a pre-clinical mouse model. The Cas9-competent MOLM-13 cells were labeled with luciferase (Luci) and transduced them with sgRNA targeting CLPB or negative control (sgRosa). sgRNA-infected MOLM-13 (GFP+) were then sorted by FACS and injected intravenously into NSG mice (FIG. 12D). A significant delay in leukemia progression was observed in mice receiving CLPB-deficient MOLM-13 cells, as determined by bioluminescent imaging (FIGS. 12E, 12F). FACS analysis of peripheral blood from recipient mice detected a significant lower percentage of circulating leukemia cells harboring CLPB sgRNAs compared to the negative control (FIG. 12G), which correlated with the significant prolonged survival seen in mice harboring CLPB-deficient MOLM-13 (FIG. 12H). These data suggested that CLPB is required for AML maintenance, which is consistent with the physiological function of CLPB in regulating key mitochondrial biological processes, including cristae structure, membrane potential and oxidative phosphorylation.

### Example 8—Loss of CLPB Induces Mitochondrial Stress Response

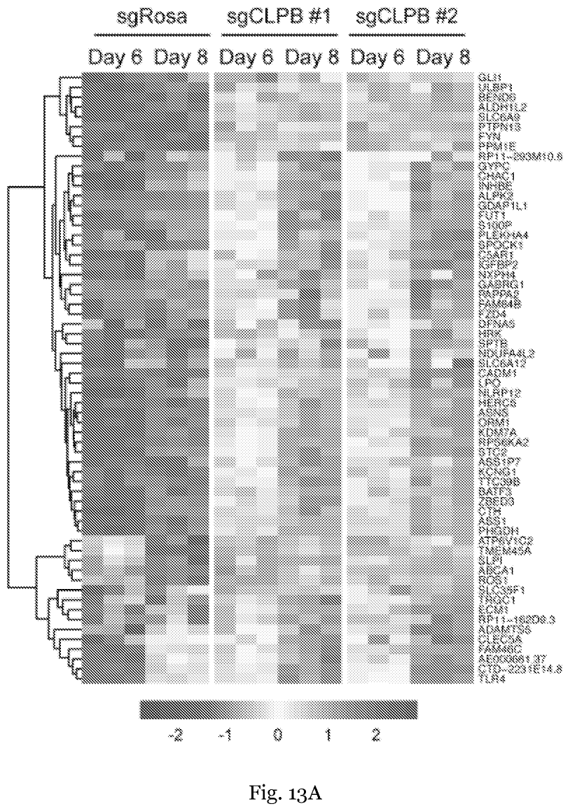

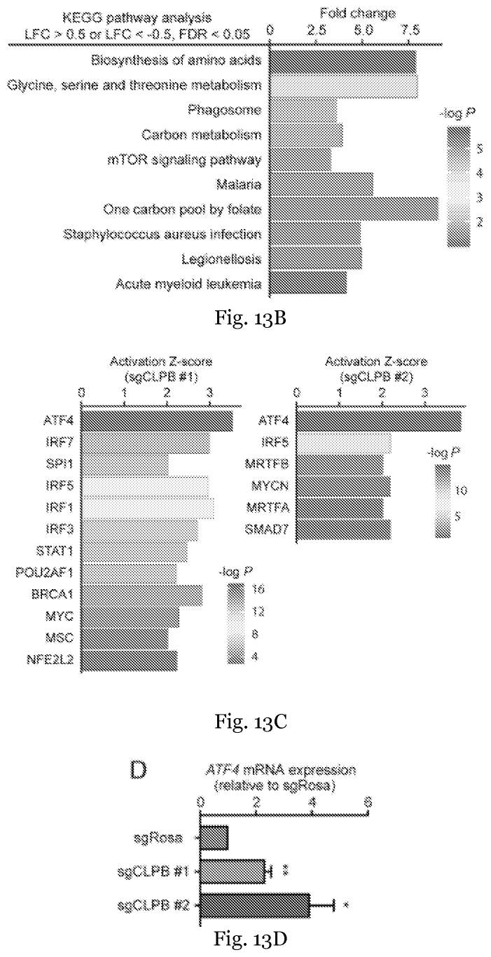

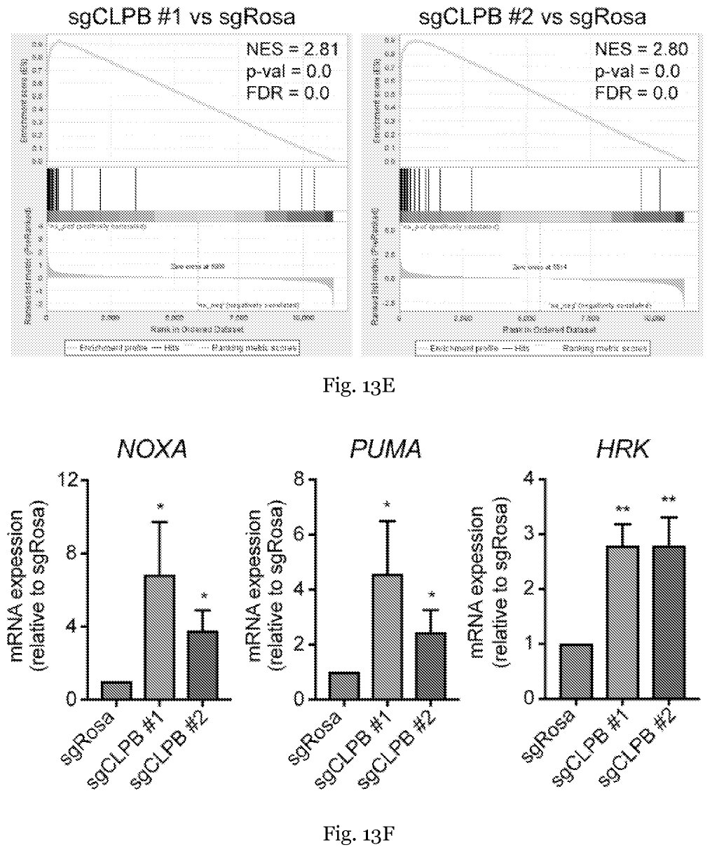

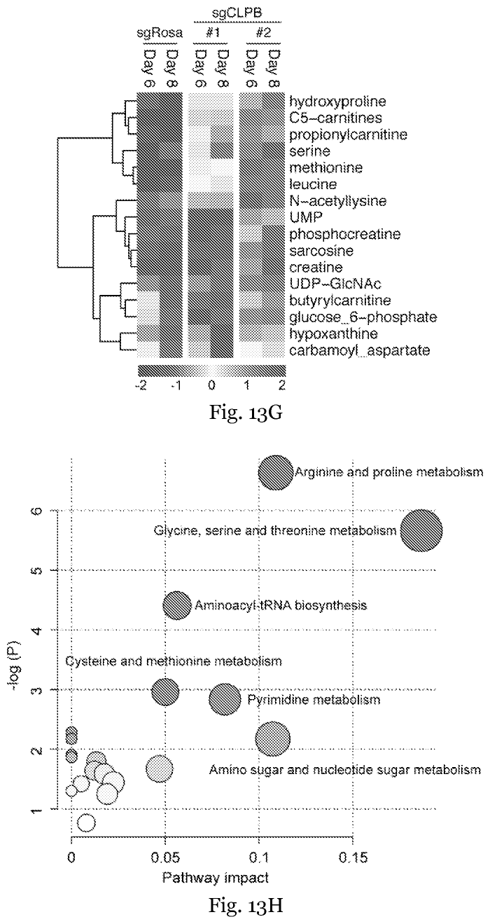

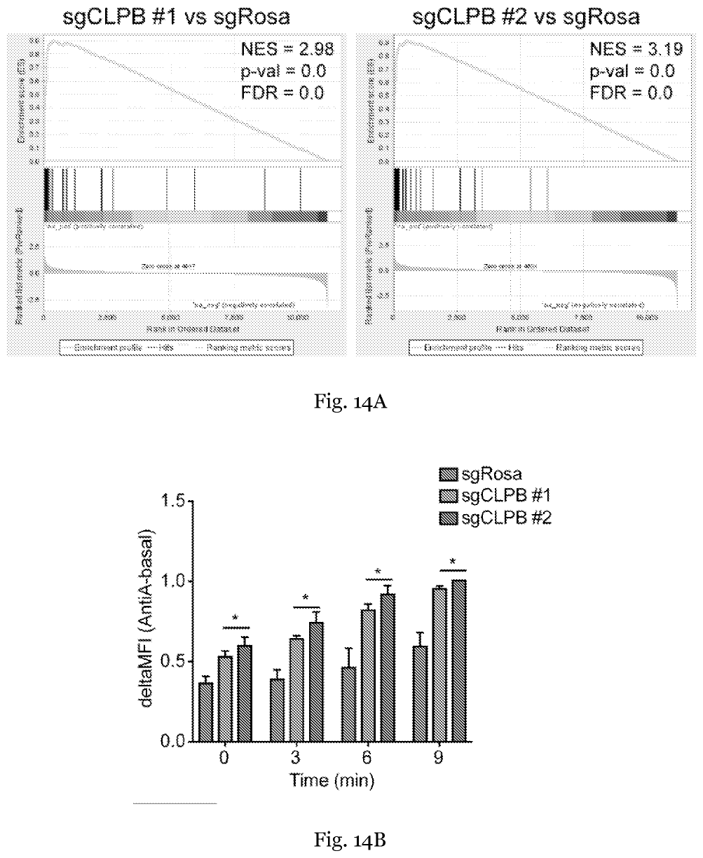

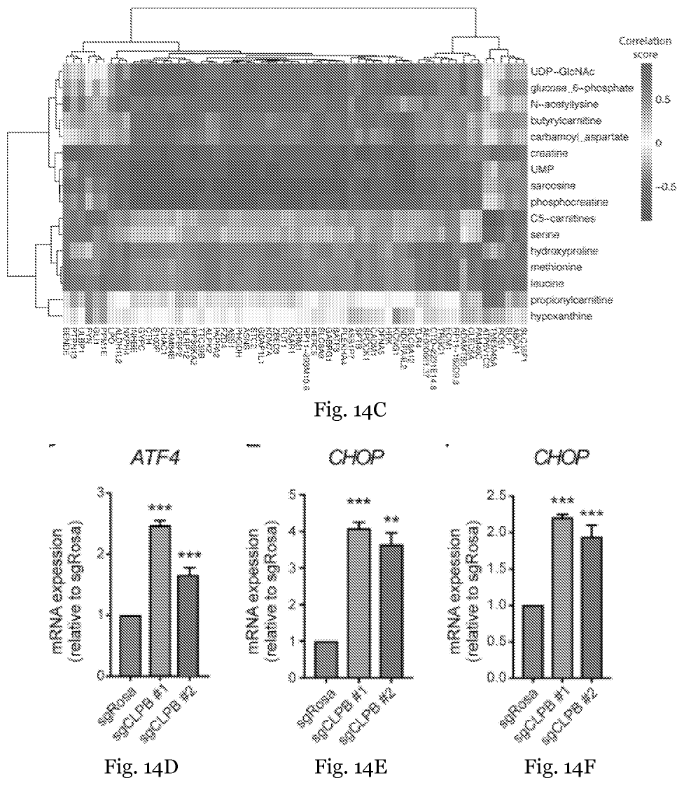

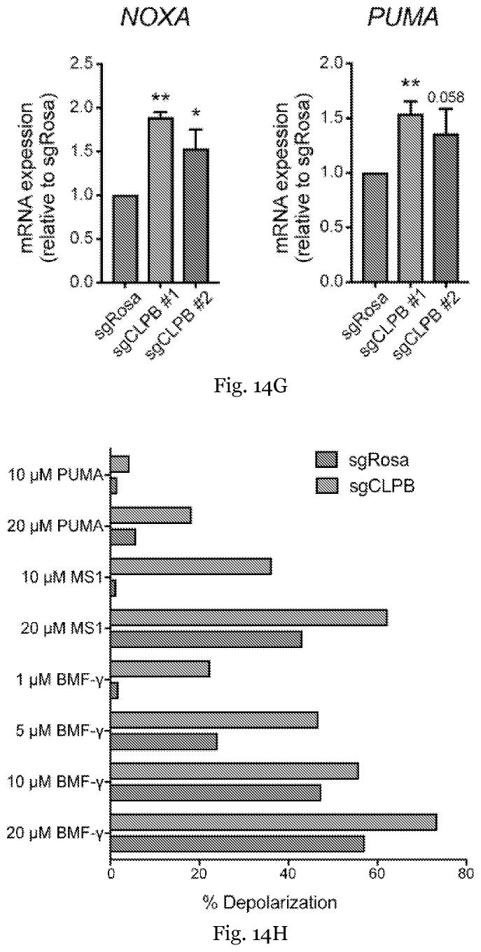

To further focus on the underlying mechanisms of CLPB function, RNA-Sequencing was performed in AML cells upon CLPB deletion. Two CLPB-targeting sgRNAs were used and cells were profiled at days 6 and 8 after CLPB depletion (FIG. 13A). KEGG pathway analysis of the differentially expressed genes in both CLPB-deficient MOLM-13 cells (sgCLPB #1 and #2) showed strong enrichment on metabolic pathways, like biosynthesis of amino acids and one carbon metabolism (FIG. 13B), all consistent with the functions of CLPB in regulating mitochondrial processes (FIGS. 12A-12H). Ingenuity Pathway Analysis (IPA) was performed to reveal the upstream transcriptional regulators that are responsible for the transcriptional landscape changes upon CLPB ablation. Interestingly, ATF4, a key transcription factor induced by the integrative stress response (Wortel et al., “Surviving Stress: Modulation of ATF4-Mediated Stress Responses in Normal and Malignant Cells,” Trends Endocrinol. Metab. 28:794-806 (2017), which is hereby incorporated by reference in its entirety), ranked first in both CLPB-targeting sgRNAs transduced MOLM-13 cells (FIG. 13C). Indeed, qPCR analysis verified the upregulation of ATF4 and its downstream effector CHOP in AML cells lacking CLPB (FIGS. 13D, 14D-14F). Considering the previous results and given that ATF4 has been identified as a key regulator of the mitochondrial stress response in mammals (Quiros et al., “Multi-Omics Analysis Identifies ATF4 as a Key Regulator of the Mitochondrial Stress Response in Mammals,” J. Cell Biol. 216:2027-45 (2017), which is hereby incorporated by reference in its entirety), it was speculated that CLPB-deficient AML cells are under mitochondrial stress. To this end, Gene-Set Enrichment Analysis (GSEA) was performed using a combined mitochondrial stress expression gene signature extracted from the RNA-Seq studies described by Quirós et al (Quiros et al., “Multi-Omics Analysis Identifies ATF4 as a Key Regulator of the Mitochondrial Stress Response in Mammals,” J. Cell Biol. 216:2027-45 (2017), which is hereby incorporated by reference in its entirety). Such analysis revealed that genes associated with mitochondrial stress response were indeed strongly enriched in CLPB-deficient MOLM-13 cells at both time-points (FIGS. 13E, 14A). To functionally confirm the mitochondrial stress in CLPB-deficient AML cells, mitochondrial reactive oxygen species (ROS) was measured upon treatment with the complex III inhibitor, Antimycin A, using MitoSOX Red. CLPB deficiency in MOLM-13 led to a significant elevation of the mean MitoSOX fluorescent intensity after Antimycin A addition relative to the steady-state levels, indicating greater ROS accumulation inside CLPB ablated mitochondria upon challenge (FIG. 14B), thus, demonstrating that AML cells are under mitochondrial stress upon CLPB depletion. Given that proapoptotic BH3-only proteins can be induced by ATF4 (Pike et al., “ATF4 Orchestrates a Program of BH3-Only Protein Expression in Severe Hypoxia,” Mol Biol Rep. 39:10811-22 (2012), which is hereby incorporated by reference in its entirety), it was wondered if these pro-death sensitizers were upregulated in response to CLPB depletion. Indeed, NOXA, PUMA and HRK gene expression was significantly upregulated in CLPB-deficient AML cells (FIGS. 13F, 14G). In line with this finding, BH3-profiling revealed increased sensitivity of CLPB-ablated cells to the peptide “sensitizers” PUMA and BMF-v, as well as to the MCL-1 inhibitor (MS-1) (FIG. 14H). Such findings were also consistent with the previous results showing that CLPB ablated AML cells are more primed to apoptosis (FIGS. 11G, 11H).

Considering that CLPB-deficient AML cells are under mitochondrial stress and harbor dysfunction in the biosynthesis of several metabolites (FIG. 13B), a more detailed metabolic profiling was performed using mass spectroscopy. Among the 150 metabolites tested, the intensity of 16 substances was significantly changed in CLPB-deficient MOLM-13 cells (FIG. 13G). Pathway analysis of the metabolomics data revealed significant enrichment in amino acid related pathways (glycine, serine and threonine metabolism, arginine and proline metabolism, aminoacyl-tRNA biosynthesis) (FIG. 13H). Indeed, the glycine, serine and threonine biosynthesis is one of the most altered metabolic pathways in cells under mitochondrial stress (Quiros et al., “Multi-Omics Analysis Identifies ATF4 as a Key Regulator of the Mitochondrial Stress Response in Mammals,” J. Cell Biol. 216:2027-45 (2017), which is hereby incorporated by reference in its entirety). Notably, the majority of the altered metabolites upon CLPB depletion were strongly correlated with the differentially expressed genes shown in FIG. 13A, highlighting the consistency of the RNA-Seq and metabolomics data (FIG. 14C). These findings suggest that CLPB deficiency amplifies proapoptotic signals through the induction of the ATF4-mediated mitochondrial stress response..

### Example 9—CLPB Targeting Overcomes p53-Mediated Venetoclax Resistance and Sensitizes AML Cells to Combined Venetoclax and Azacitidine Treatment

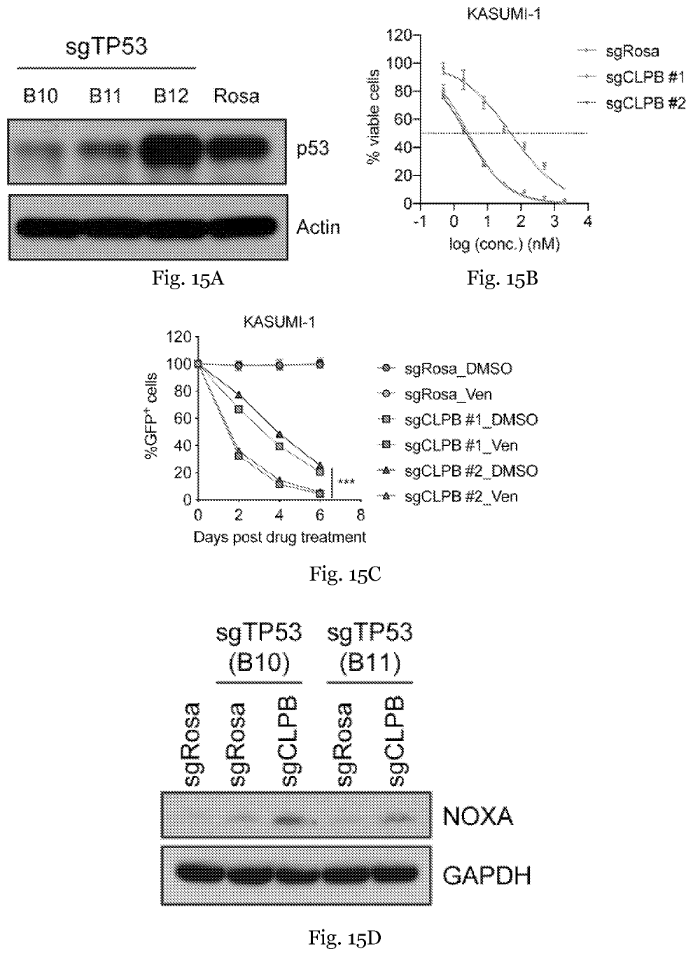

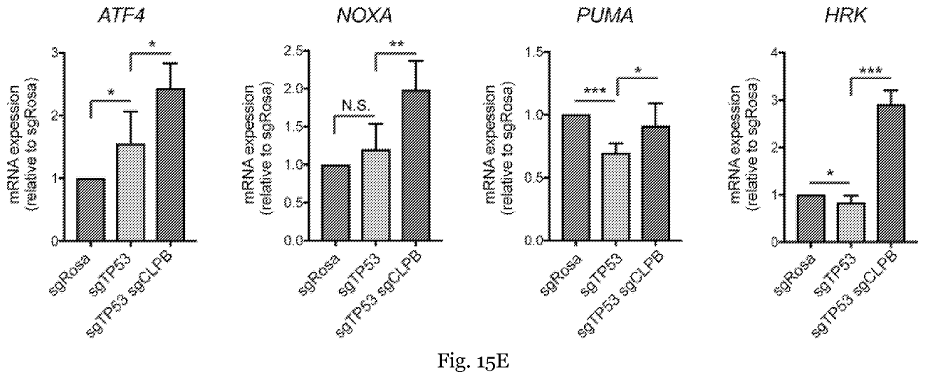

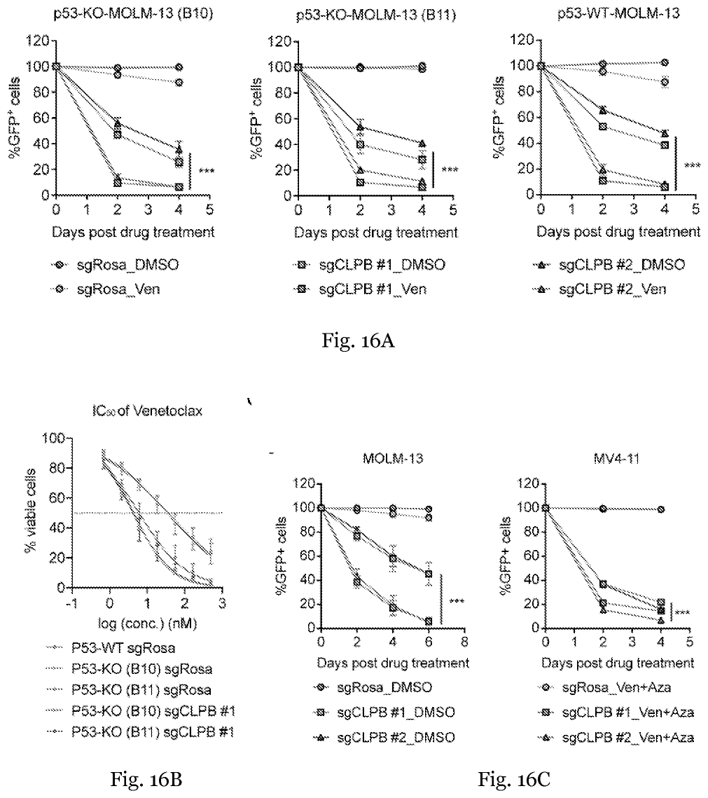

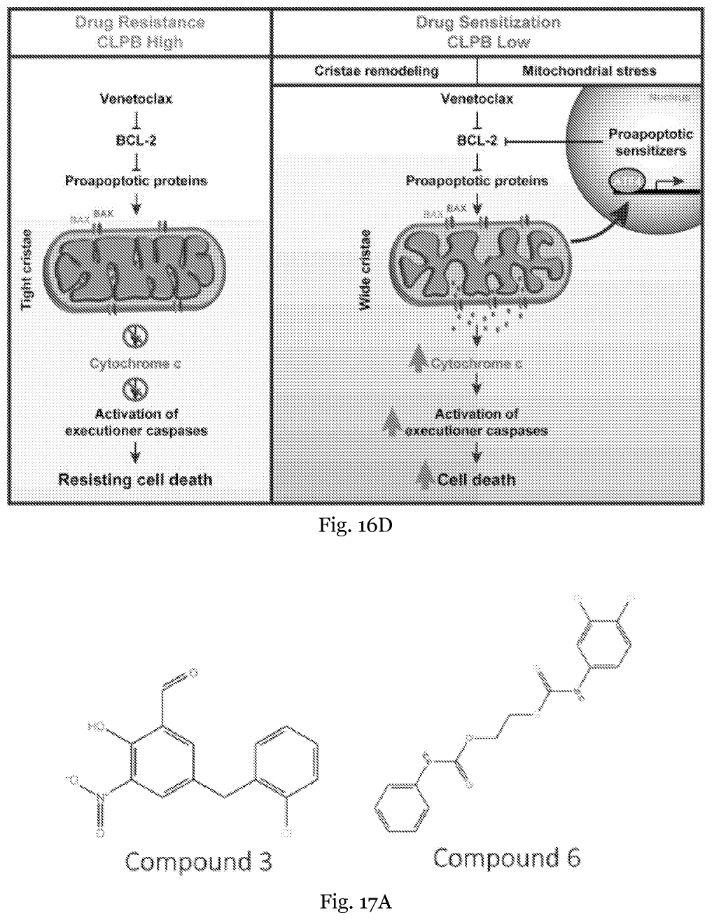

Stabilization of the p53 protein synergizes with Venetoclax in AML (Pan et al., “Synthetic Lethality of Combined Bcl-2 Inhibition and p53 Activation in AML: Mechanisms and Superior Antileukemic Efficacy,” Cancer Cell 32:748-60 e6 (2017), which is hereby incorporated by reference in its entirety). Based on data included here and in the accompanying manuscript by Tyner and colleagues (Nechiporuk et al., “The TP53 Apoptotic Network is a Primary Mediator of Resistance to BCL2 inhibition in AML Cells,” Cancer Discovery 9(7):910-925 (2019), which is hereby incorporated by reference in its entirety), p53 deletion can confer resistance to the drug in AML cells (FIGS. 1B-1E, 2B, 2D-2F). Therefore, the key question of obvious clinical significance is whether CLPB deletion can sensitize p53-deficient AML cells to Venetoclax treatment. To this end, p53 was deleted in MOLM-13 using two independent sgRNAs targeting TP53 (FIG. 15A). The studies described here showed that CLPB-deficient AML cells were more sensitive to Venetoclax treatment, even in a TP53-knockout (p53-KO) background (FIG. 16A). More importantly, CLPB depletion was able to fully re-sensitize p53-deficient MOLM-13 to Venetoclax treatment by inducing the ATF4-mediated upregulation of proapoptotic sensitizers (FIGS. 16B, 15D, 15E). Likewise, CLPB ablation led to a synergistic effect with Venetoclax also in an AML cell line with naturally occurring TP53 mutation (KASUMI-1) (FIGS. 15B, 15C). Collectively, CLPB-mediated re-sensitization of Venetoclax-resistant AML cells is p53-independent.

Since resistance to Venetoclax monotherapy rapidly ensues in AML (Tahir et al., “Potential Mechanisms of Resistance to Venetoclax and Strategies to Circumvent it,” BMC Cancer 17:399 (2017); and Bose et al., “Pathways and Mechanisms of Venetoclax Resistance,” Leuk. Lymphoma 58:1-17 (2017), which are each hereby incorporated by reference in their entirety), multiple combinational therapies for Venetoclax have been proposed and tested, some of which are in clinical trials. Among these, the combination of Venetoclax with hypomethylating agents (HMA), like azacitidine, is currently in clinics due to its favorable responses in the clinical trials (DiNardo et al., “Venetoclax Combined with Decitabine or Azacitidine in Treatment-Naive, Elderly Patients with Acute Myeloid Leukemia,” Blood 133:7-17 (2019), which is hereby incorporated by reference in its entirety). To investigate if CLPB inhibition can enhance the efficacy of Venetoclax and Azacitidine combined treatment, the possibility that CLPB depletion can further sensitize AML cells to the combined treatment was tested. Indeed, CLPB-deficient AML cells were negatively selected out upon combined treatment in a much faster pace than the DMSO control groups (FIG. 16C), confirming that CLPB ablation can sensitize AML cells to combined Venetoclax/Azacitidine treatment. Altogether, these findings highlight that loss of the mitochondrial protein CLPB leads to structural and functional defects of mitochondria, hence sensitizing AML cells to apoptosis. Therefore, targeting CLPB synergizes with Venetoclax and Venetoclax/Azacitidine combination in AML in a p53-independent manner (FIG. 16D).

### Discussion of Examples 1-9

The recent FDA approval for Venetoclax introduces an exciting and novel strategy to target AML. In the initial single-agent Venetoclax studies in AML, response rates were modest and rather short-lived (Konopleva et al., “Efficacy and Biological Correlates of Response in a Phase II Study of Venetoclax Monotherapy in Patients with Acute Myelogenous Leukemia,” Cancer Discov. 6:1106-17 (2016), which is hereby incorporated by reference in its entirety). The genome-wide CRISPR/Cas9 screen identified both possible mechanisms of resistance and synthetic lethal combinations with Venetoclax in AML, including the targeting of a number of proteins regulating mitochondrial structure. These proteins can control the architecture of both the outer mitochondrial membrane (OMM) and the mitochondrial cristae. Interestingly, both these structures are essential for BCL-2 family function (Antignani et al., “How do Bax and Bak Lead to Permeabilization of the Outer Mitochondrial Membrane?,” Curr. Opin. Cell Biol. 18:685-9 (2006), which is hereby incorporated by reference in its entirety), cytochrome c release and cell death (Scorrano et al., “Mechanisms of Cytochrome c Release by Proapoptotic BCL-2 Family Members,” Biochem. Biophys. Res. Commun. 304:437-44 (2003), which is hereby incorporated by reference in its entirety). For the complete release of the cristae-endowed cytochrome c stores, the inner mitochondrial membrane needs to be remodeled and the cristae widened by the proteolytic cleavage of OPA1 and the disruption of its complexes, which normally keep two opposing cristae membranes tight (Frezza et al., “OPA1 Controls Apoptotic Cristae Remodeling Independently from Mitochondrial Fusion,” Cell 126:177-89 (2006); and Scorrano et al., “A Distinct Pathway Remodels Mitochondrial Cristae and Mobilizes Cytochrome c During Apoptosis,” Dev. Cell 2:55-67 (2002), which are each hereby incorporated by reference in their entirety). Both OMM rupture and cristae remodeling is what we observed by electron microscopy upon Venetoclax treatment in AML cells. The changes in mitochondrial architecture could, at least partially, explain the reported impairment in oxidative phosphorylation following BCL-2 inhibition in the leukemic stem cells (LSC) derived from AML patients (Lagadinou et al., “BCL-2 Inhibition Targets Oxidative Phosphorylation and Selectively Eradicates Quiescent Human Leukemia Stem Cells,” Cell Stem Cell 12:329-41 (2013), which is hereby incorporated by reference in its entirety). On the opposite side, Venetoclax-resistant AML cells display mitochondria with narrow cristae lumen and increased protein levels of the cristae “staple” OPA1, possibly rendering them more resistant to the cytochrome c release upon stimulation.

One of the top-ranked genes revealed by the screen to act synergistically with Venetoclax is the mitochondrial chaperonin CLPB. Although a plethora of studies in bacteria and yeast have extensively characterized the homologue of CLPB as a protein chaperone which disaggregates misfolded proteins (Lee et al., “The Structure of ClpB: a Molecular Chaperone that Rescues Proteins from an Aggregated State,” Cell 115:229-40 (2003); Rottgers et al., “The ClpB homolog Hsp78 is Required for the Efficient Degradation of Proteins in the Mitochondrial Matrix,” J. Biol. Chem. 277:45829-37 (2002); and Abrahao et al., “Hsp78 (78 kDa Heat Shock Protein), a Representative AAA Family Member Found in the Mitochondrial Matrix of Saccharomyces cerevisiae,” Front Mol. Biosci. 4:60 (2017), which are each hereby incorporated by reference in their entirety), the exact function of the mammalian CLPB has not been investigated thoroughly. Of note, the amino-acid sequence of the human CLPB is only about 20% identical to the orthologue in Escherichia coli (Abrahao et al., “Hsp78 (78 kDa Heat Shock Protein), a Representative AAA Family Member Found in the Mitochondrial Matrix of Saccharomyces cerevisiae,” Front Mol. Biosci. 4:60 (2017), which is hereby incorporated by reference in its entirety). CLPB belongs to the large AAA+ ATPase superfamily, whose members act both as general protein chaperones and as targeted proteases that degrade specific substrates. Other mitochondrial AAA+ ATPases, such as YME1L, AFG3L2, ATAD3, m-AAA protease, participate in the organelle's protein quality control, mitochondrial protein synthesis and maintenance of mitochondrial architecture (Gerdes et al., “Mitochondrial AAA Proteases-Towards a Molecular Understanding of Membrane-Bound Proteolytic Machines,” Biochim. Biophys. Acta. 1823:49-55 (2012); Glynn, “Multifunctional Mitochondrial AAA Proteases,” Front Mol. Biosci. 4:34 (2017); Gilquin et al., “The AAA+ ATPase ATAD3A Controls Mitochondrial Dynamics at the Interface of the Inner and Outer Membranes,” Mol. Cell Biol. 30:1984-96 (2010); Anand et al., “The i-AAA Protease YME1L and OMA1 Cleave OPA1 to Balance Mitochondrial Fusion and Fission,” J. Cell Biol. 204:919-29 (2014); and Almajan et al., “AFG3L2 Supports Mitochondrial Protein Synthesis and Purkinje Cell Survival,” J. Clin. Invest. 122:4048-58 (2012), which are each hereby incorporated by reference in their entirety). Wortmann and colleagues, who studied the mutations in CLPB in patients with an autosomal recessive metabolic syndrome, hypothesized a possible role of CLPB in apoptosis, due to its in situ predicted interaction with HAX1 (Wortmann et al., “CLPB Mutations Cause 3-Methylglutaconic Aciduria, Progressive Brain Atrophy, Intellectual Disability, Congenital Neutropenia, Cataracts, Movement Disorder,” Am. J. Hum. Genet. 96:245-57 (2015), which is hereby incorporated by reference in its entirety). The study described herein provides the biochemical evidence of the association of CLPB with HAX1 and uncovers its function in the cell protection from intrinsic mitochondria-mediated apoptosis. Furthermore, this work reveals that CLPB physically interacts with OPA1—possibly as a means to protect it from its proteolytic cleavage—and, thus, demonstrates a role of CLPB in the maintenance of the physiological mitochondrial structure. Loss of CLPB results in abnormal mitochondria with wider cristae than the normal; hence such organelles are more prone to release their cytochrome c following a death signal, such as Venetoclax treatment. This work proposes a specific CLPB dependency of AML cells. Its depletion leads to cell cycle suppression, possibly due to the defects in both oxidative phosphorylation and glycolysis. As a consequence of the mitochondrial structural and functional impairment, cells lacking CLPB are more sensitive to oxidative stress. As a response, the CLPB-deficient AML cells alter their transcriptional program and reprogram their metabolism in an ATF4-dependent manner, thus leading to cell cycle arrest and enhancement of apoptotic pathways. Overall, this study shows that CLPB is most likely required for AML progression and, importantly, can synergize with Venetoclax to induce rapid cell death both in vivo and in vitro.

Recently, Jordan and colleagues demonstrated that the FDA approved combined treatment of Venetoclax with Azacitidine disrupts the tricarboxylic acid (TCA) cycle in LSCs of AML patients and that relapsed AML patients remodel their metabolism (Pollyea et al., “Venetoclax with Azacitidine Disrupts Energy Metabolism and Targets Leukemia Stem Cells in Patients with Acute Myeloid Leukemia,” Nat. Med. 24:1859-66 (2018); and Jones et al., “Inhibition of Amino Acid Metabolism Selectively Targets Human Leukemia Stem Cells,” Cancer Cell 34:724-40 e4 (2018), which are each hereby incorporated by reference in their entirety). These studies support the notion that targeting mitochondrial functions concomitantly with Venetoclax and Azacitidine could have a clinical relevance for AML patients.v In line with this idea, the study described herein shows that depleting CLPB which impacts on the cellular metabolic status of the AML cells sensitizes them to the combined treatment in a p53-independent way. Interestingly, a bacterial CLPB inhibitor has been developed and proposed to be utilized as an antimicrobial agent (Martin et al., “Screening and Evaluation of Small Organic Molecules as ClpB Inhibitors and Potential Antimicrobials,” J. Med Chem. 56:7177-89 (2013), which is hereby incorporated by reference in its entirety). Since this inhibitor targets the conserved domain of the bacteria and the human orthologues (Wortmann et al., “CLPB Mutations Cause 3-Methylglutaconic Aciduria, Progressive Brain Atrophy, Intellectual Disability, Congenital Neutropenia, Cataracts, Movement Disorder,” Am. J. Hum. Genet. 96:245-57 (2015), which is hereby incorporated by reference in its entirety) and displays moderate toxicity in human cell lines (Martin et al., “Screening and Evaluation of Small Organic Molecules as ClpB Inhibitors and Potential Antimicrobials,” J. Med. Chem. 56:7177-89 (2013), which is hereby incorporated by reference in its entirety), it could synergize with Venetoclax treatment in AML cells. In general, targeting mitochondrial proteins that would promote apoptotic cristae remodeling should be investigated as a potential way to overcome Venetoclax resistance in AML, in addition to ongoing trials targeting other BCL-2 family members, such as MCL-1, either directly with novel MCL-1 inhibitors, or indirectly via cyclin dependent kinase inhibition affecting MCL-1 transcript expression, as has been proposed (Bogenberger et al., “BCL-2 Family Proteins as 5-Azacytidine-Sensitizing Targets and Determinants of Response in Myeloid Malignancies,” Leukemia 28:1657-65 (2014); and Bogenberger et al., “Combined Venetoclax and Alvocidib in Acute Myeloid Leukemia,” Oncotarget. 8:107206-22 (2017), which are each hereby incorporated by reference in their entirety). In conclusion, targeting CLPB and possibly its interactome in combination with Venetoclax treatment directly promotes the apoptotic cristae remodeling and indirectly induces mitochondrial stress responses, which will both amplify the programmed cell death pathway and suppress the growth of AML cells. These data open a potential avenue for novel Venetoclax drug combinations in AML.

### Materials and Methods for Examples 1-9

Cell Lines and Cell Culture. All human leukemia cells (MOLM-13, THP-1, MV4-11, CuTLL-1, Nalm-6, K562) were cultured in RPMI medium supplemented with 10% FBS and 1% penicillin/streptomycin. The adherent cell lines, HeLa and HEK293T cells were grown in DMEM medium with 10% FBS and 1% penicillin streptomycin. SK-MEL-239 were cultured in RPMI, 100% FBS and 1% penicillin/streptomycin. Cas9-expressing cell lines transduced with retroviral Cas9-2A-blast (Addgene, plasmid no. 73310) were selected with blasticidin (InvivoGen) 48 hours after transduction. All transfections were performed in HEK293T cells using Polyethylenimine (PEI) reagent at 4:2:3 ratios of sgRNA or shRNA construct (Tables 2A-2C): pVSVG: pPax2 in OPTI-MEM solution. Viral supernatant was collected 36 hrs and 48 hrs post-transfection. Spin infections were performed at room temperature at 1,500×g for 30 mins with polybrene reagent (1:2000; Fisher Scientific).

TABLE 2A
List of shRNAs and paired shRNAs used in this study

| | | SEQ | forward oligo | SEQ | reverse oligo | SEQ |
|---|---|---|---|---|---|---|
| sgRNA | | ID | with overhangs | ID | with overhangs | ID |
| name | sgRNA sequence | NO: | (5′-3′) | NO: | (5′-3′) | NO: |

List of sgRNAs used in this study

| | | | | | | |
|---|---|---|---|---|---|---|
| sgCLPB #1 | GTTTGGCCGTA | 4 | CACCGGTTTGG | 5 | AAACCCGGAT | 6 |
| | GCGATCCGG | | CCGTAGCGAT | | CGCTACGGCC | |
| | | | CCGG | | AAACC | |
| sgCLPB #2 | GTACGATGAA | 7 | CACCGGTACG | 8 | AAACGAGGGT | 9 |
| | GAACACCCTC | | ATGAAGAACA | | GTTCTTCATCG | |
| | | | CCCTC | | TACC | |
| sgBAX #1 | AGCGAGTGTCT | 10 | CACCGAGCGA | 11 | AAACATGCGC | 12 |
| | CAAGCGCAT | | GTGTCTCAAGC | | TTGAGACACTC | |
| | | | GCAT | | GCTC | |
| sgBAX #2 | AGTAGAAAAG | 13 | CACCGAGTAG | 14 | AAACGGTTGTC | 15 |
| | GGCGACAACC | | AAAAGGGCGA | | GCCCTTTTCTA | |
| | | | CAACC | | CTC | |
| sgPMAIP1 | TTTGCTTTCCT | 16 | CACCGTTTGCT | 17 | AAACGCTCTG | 18 |
| #1 | TCTCAGAGC | | TTCCTTCTCAG | | AGAAGGAAAG | |
| | | | AGC | | CAAAC | |
| sgPMAIP1 | ACGCTCAACC | 19 | CACCGACGCT | 20 | AAACCGCGGG | 21 |
| #2 | GAGCCCCGCG | | CAACCGAGCC | | GCTCGGTTGAG | |
| | | | CCGCG | | CGTC | |
| sgTP53_#1 | CCATTGTTCAA | 22 | CACCGCCATTG | 23 | AAACCGGACG | 24 |
| (B10) | TATCGTCCG | | TTCAATATCGT | | ATATTGAACA | |
| | | | CCG | | ATGGC | |
| sgTP53_#2 | GAGCGCTGCT | 25 | CACCGAGCGC | 26 | AAACTCGCTAT | 27 |
| (B11) | CAGATAGCGA | | TGCTCAGATAG | | CTGAGCAGCG | |
| | | | CGA | | CTC | |
| sgSLC25A1 | CTGCGTCTTCA | 28 | CACCGCTGCGT | 29 | AAACCCGAGT | 30 |
| #1 | CGTACTCGG | | CTTCACGTACT | | ACGTGAAGAC | |
| | | | CGG | | GCAGC | |
| sgSLC25A1 | GAACTCGAAC | 31 | CACCGGAACT | 32 | AAACGGTTTG | 33 |
| #2 | ATTCCAAACC | | CGAACATTCC | | GAATGTTCGA | |
| | | | AAACC | | GTTCC | |
| sgRTN4IP1 | GAGAGCCACA | 34 | CACCGAGAGC | 35 | AAACCTTTGCC | 36 |
| #1 | TATGGCAAAG | | CACATATGGC | | ATATGTGGCTC | |
| | | | AAAG | | TC | |
| sgRTN41P1 | GGGTCGGGAT | 37 | CACCGGGTCG | 38 | AAACCGCCAG | 39 |
| #2 | GTCTCTGGCG | | GGATGTCTCTG | | AGACATCCCG | |
| | | | GCG | | ACCC | |
| sgGID8 #1 | ATCCGGGAGA | 40 | CACCGATCCG | 41 | AAACTTCAGTA | 42 |
| | TGATACTGAA | | GGAGATGATA | | TCATCTCCCGG | |
| | | | CTGAA | | ATC | |
| sgG1D8 #2 | GTAAGCCATTA | 43 | CACCGTAAGC | 44 | AAACGGTCAC | 45 |
| | CCTGTGACC | | CATTACCTGTG | | AGGTAATGGC | |
| | | | ACC | | TTAC | |
| sgRosa | AACGGCTCCA | 46 | CACCGAACGG | 47 | AAACCCGAGC | 48 |
| | CCACGCTCGG | | CTCCACCACGC | | GTGGTGGAGC | |
| | | | TCGG | | CGTTC | |

List of paired sgRNAs used in this study

| | |
|---|---|
| BAX/BAK | CATGCGAGAAAAGCCTTGTTTGGGCTCACCTGCTAGGTTGCAGGTT |
| sgRNA #1 | TGGGTCTTCGAGAAGACCTCACCGGTTTCATCCAGGATCGAGCAGT |
| (SEQ ID | TTTAGAGCTAGAAATAGC |
| NO: 49) | |

TABLE 2B
List of primers for qPCR used in this study

| Primer | Name | Sequence (5′- 3′) | SEQ ID NO: |
|---|---|---|---|
| ATF4 | FRW | TGACCTGGAAACCATGCCAG | 50 |
| ATF4 | RVS | AATGATCTGGAGTGGAGGAC | 51 |
| CHOP | FRW | CAGAACCAGCAGAGGTCACA | 52 |
| CHOP | RVS | AGCTGTGCCACTTTCCTTTC | 53 |
| PMAIP1 | FRW | GCGCAAGAACGCTCAACC | 54 |
| PMAIP1 | RVS | TCAGGTTCCTGAGCAGAAGAG | 55 |
| BBC3 | FRW | GACCTCAACGCACAGTACGAG | 56 |
| BBC3 | FRW | AGGAGTCCCATGATGAGATTGT | 57 |
| HRK | FRW | AGCGAGCAACAGGTTGGTGA | 58 |
| HRK | RVS | CACTTCCTTCTCGAAGTGCCA | 59 |
| beta-ACTIN | FRW | GATCATTGCTCCTCCTGAGC | 60 |
| beta-ACTIN | RVS | ACATCTGCTGGAAGGTGGAC | 61 |

TABLE 2C
List of shRNAs used in this study

| | | SEQ |
|---|---|---|
| | | ID |
| shRNA ID | shRNA sequence (5′-3′) | NO: |
| shCLPB #1 | TGCTGTTGACAGTGAGCGACAGGTGCAGATAGT | 62 |
| | TCTGCAATAGTGAAGCCACAGATGTATTGCAGA | |
| | ACTATCTGCACCTGGTGCCTACTGCCTCGGA | |
| shCLPB #2 | TGCTGTTGACAGTGAGCGAACGGATCAATGAGA | 63 |
| | TCGTCTATAGTGAAGCCACAGATGTATAGACGA | |
| | TCTCATTGATCCGTCTGCCTACTGCCTCGGA | |
| shCLPB #3 | TGCTGTTGACAGTGAGCGAACGGATCAATGAGA | 64 |
| | TCGTCTATAGTGAAGCCACAGATGTATAGACGA | |
| | TCTCATTGATCCGTCTGCCTACTGCCTCGGA | |
| shCLPB #4 | TGCTGTTGACAGTGAGCGATCCAGATAAGTGAC | 65 |
| | AAGATCATAGTGAAGCCACAGATGTATGATCTT | |
| | GTCACTTATCTGGACTGCCTACTGCCTCGGA | |

For the generation of the Venetoclax-resistant cell lines, MOLM-13 and MV4-11 were cultured in media containing increasing doses of Venetoclax (from 5 nM to 1000 nM) for more than 8 weeks to achieve complete resistance to the drug.

Cell lines information is as follows: MOLM-13 (DSMZ, ACC554), THP-1, (ATCC, TIB-202), MV4-11 (ATCC, CRL-9591), CuTLL-1 (Adolfo Ferrando Lab), Nalm-6 (ATCC, CRL-3273), K562 (ATCC, CCL-243), HEK293T (ATCC, CRL-1573), SK-MEL-239 (Eva Hernando Lab), HeLa (ATCC, CCL-2).

All the cell lines were determined negative for mycoplasma, as indicated by the latest Mycoplasma test (December 2018) using the LookOut Mycoplasma PCR Detection Kit (Sigma). Cells were used for experiments within 15 to 20 passages from thawing.

CRISPR Screen. Cas9-expressing MOLM-13 were transduced with the Brunello sgRNA library (Doench et al., “Optimized sgRNA Design to Maximize Activity and Minimize Off-Target Effects of CRISPR-Cas9,” Nat. Biotechnol. 34:184-91 (2016), which is hereby incorporated by reference in its entirety) virus at a low MOI (˜0.3). On day 2 post-transduction, GFP⁺ percentage was assessed to determine infection efficiency and sgRNA coverage (˜1000×). Then, puromycin (1 μg/ml) was added for 5 days to select infected (GFP⁺) cells. After selection, viable infected cells were isolated by Histopaque 1077 (Sigma Aldrich), and grown without antibiotics for 2 days. After recovery, 100×10⁶ infected cells were cultured with 10 nM Venetoclax (Selleckchem) or DMSO. Another 100×10⁶ cells were used for genomic DNA (gDNA) extraction and served as an initial reference (day 0 of drug treatment). The concentration of Venetoclax was increased during the screen (from 10 nM to 2 μM) to avoid spontaneous gain of drug resistance. For each passage, 100×10⁶ cells were placed back into culture until 16 days post drug treatment. gDNA of cells containing ˜1000× coverage was harvested on days 8 and 16 post drug treatment using Qiagen DNA kit according to manufacturer's protocol.

Drug Treatment and IC₅₀ Measurements. Cells were plated in 96-well plates and exposed to Venetoclax at concentrations ranging from 0.4 nM to 300 nM (for MOLM-13 and MV4-11), 150 nM to 10 μM (for Venetoclax-resistant cell lines and THP-1) with a minimum of three technical replicates per concentration per cell line. Cell viability was measured with the CellTiter-Glo reagent (Promega) according to manufacturer's instructions. Absolute viability values were converted to percentage viability versus DMSO control treatment, and then non-linear fit of log(inhibitor) versus response (three parameters) was performed in GraphPad Prism v7.0 to obtain the IC₅₀ values. For Venetoclax and Azacitidine combined treatment, two drugs were mixed at the fixed ratio (Ven:Aza=1:4) before diluting to certain concentrations.

Transmission Electron Microscopy. Cultured cells were fixed in 0.1M sodium cacodylate buffer (pH 7.4) containing 2.5% glutaraldehyde and 2% paraformaldehyde for 2 hrs and post-fixed with 1% osmium tetroxide and 1% potassium ferrocyanide for one hour at 4° C. Then block was stained in 0.25% aqueous uranyl acetate, processed in a standard manner and embedded in EMbed 812 (Electron Microscopy Sciences, Hatfield, Pa.). Ultrathin sections (60 nm) were cut, mounted on copper grids and stained with uranyl acetate and lead citrate. Stained grids were examined under Philips CM-12 electron microscope and photographed with a Gatan (4k×2.7k) digital camera. For morphometric analysis, maximal cristae width was measured using Image J (NIH) as shown in FIG. 4A in at least 50 randomly selected mitochondria from a minimum of 15 cells/experiment.

Mitochondrial Respiration. Oxygen consumption rate (OCR, pmol/min) was determined using the XFe24 Extracellular Flux Analyzer and the XF Cell Mito Stress Test kit (Agilent).

Mitochondrial Membrane Potential. Mitochondrial membrane potential was monitored cytofluorimetrically using tetramethylrhodamine methyl ester (TMRM). Cells were loaded with 10 nM TMRM in presence of 2 μM Cyclosporin H in Hank's Balanced Salt Solution (HBSS) supplemented with 10 mM HEPES, 30 min at 37° C. When indicated, the experiments were performed in cells pre-treated with Venetoclax for 16 hours at the indicated concentrations.

Metabolomics. 7×10⁵ cells were flash frozen as pellets and processed using the LC-MS/MS with the hybrid metabolomics method.

Animal experiments. For in vivo experiments, Cas9 and luciferase-expressing parental and Venetoclax-resistant MOLM-13 cells were transduced with sgRosa or sgCLPB #1 constructs. At day 2 post-transduction, sgRNA positive cells (GFP) were sorted by FACS. 0.5 million sgRNA-expressing leukemia cells were intravenously injected into recipient NSG mice. For experiments that required drug administration, recipient mice were treated with vehicle or Venetoclax (100 mg/kg, Selleckchem) daily by oral gavage from day 6 to day 20. Venetoclax was prepared in 10% ethanol, 30% polyethyleneglycol-400 (Sigma), and 60% phosal 50 propylene glycol (Lipoid). Whole-body bioluminescent imaging was performed at indicated time points by intraperitoneally injection of Luciferin (Goldbio) at a 50 mg/kg concentration and imaging was performed after 5 mins using an IVIS imager. Bioluminescent signals (radiance) were quantified using Living Image software with standard regions of interests (ROI) rectangles. Peripheral blood of recipient NSG mice was collected at indicated time points after transplantation, and sgRNA positive cells (GFP+) were analyzed by flow cytometry. All animal experiments were performed in accordance with protocols approved by the New York University Institutional Animal Care and Use Committee (IACUC, ID: IA16-00008_TR1).

Data and Software Availability. Gene Expression Omnibus: all newly generated RNA-Sequencing data were deposited under accession number GSE125403.

### Supplementary Materials and Methods for Examples 1-9

CRISPR Screen and Data Analysis. For library construction, 300 μg of gDNA were amplified for 25 cycles using EX-Taq (Takara) and primer pairs that contain barcodes. PCR products were size-selected using AMPure XP beads (Beckman Coulter). Barcoded libraries were then sequenced using Next-Seq instrument (single end, 80 cycles).

Gene-based CRISPR scores were used for pooled CRISPR screen analysis as previously described (Campos et al., “High Expression of bcl-2 Protein in Acute Myeloid Leukemia Cells is Associated with Poor Response to Chemotherapy,” Blood 81:3091-6 (1993), which is hereby incorporated by reference in its entirety). Briefly, sequencing reads were aligned to the sgRNA library and the abundance of each sgRNA was calculated. The sgRNA counts from the initial populations (day 0 of drug treatment) were severed as an initial reference. sgRNAs with less than 50 counts in the initial reference were removed from downstream analyses. The log 2 fold change in abundance of each sgRNA was calculated for samples under drug treatment or DMSO treatment at day 8 and day 16 compared with the initial reference after adding a count of one as a pseudocount. log 2 fold change of each individual sgRNA was then normalized to the average log 2 fold change of the non-targeting sgRNA controls. Gene-based CRISPR scores (CS) were defined as the average normalized log 2 fold change of all sgRNAs targeting a given gene. p-value was calculated by comparing the normalized log 2 fold change of all sgRNAs targeting a given gene in Venetoclax-treated samples with those in DMSO-treated samples. Finally, the CS in the Venetoclax-treated samples were subtracted by the CS in the DMSO-treated samples to define the Delta CS (drug effect score).

The sgRNA distribution plots in FIGS. 1C and D present the spread of the delta CRISPR scores of the non-targeting controls on either day 8 or day 16. The relevant sgRNA delta CRISPR scores on either day 8 or day 16 are plotted in red over a heatmap of the corresponding non-targeting control distribution.

AML TCGA analysis. RNA-Sequencing analysis was performed using the sub-module of lncRNA-screen as previously described (Lagadinou et al., “BCL-2 Inhibition Targets Oxidative Phosphorylation and Selectively Eradicates Quiescent Human Leukemia Stem Cells,” Cell Stem Cell 12:329-41 (2013), which is hereby incorporated by reference in its entirety). Specifically, raw SRA files of 30 normal samples including 17 cord blood cells CD34+CD45RA− from GSE48846, 4 HSC and 4 MPP samples from GSE74246 and 5 healthy control CD34+ cells from GSE63569 are downloaded from SRA. Then sequencing reads are aligned to reference genome hg38 using STAR-aligner version 2.4.2 (Tzifi et al., “The Role of BCL2 Family of Apoptosis Regulator Proteins in Acute and Chronic Leukemias,” Adv. Hematol. 2012:524308 (2012), which is hereby incorporated by reference in its entirety) with parameters suggested by TCGA, and the raw read counts were generated. Raw counts of 157 TCGA AML samples are downloaded from GDC data portal. Then DESeq2 (Chao et al, “BCL-2 Family: Regulators of Cell Death,” Annu. Rev. Immunol. 16:395-419 (1998), which is hereby incorporated by reference in its entirety) was used to perform differential expression analysis between the 30 normal samples and 157 TCGA AML samples and FPKM values were generated and plotted as violin plot in FIG. 6B.

sgRNA and shRNA construction. sgRNAs were either from the Brunello sgRNA library (Antignani et al., “How do Bax and Bak Lead to Permeabilization of the Outer Mitochondrial Membrane?,” Curr. Opin. Cell Biol. 18:685-9 (2006), which is hereby incorporated by reference in its entirety) or designed manually using the “CRISPR Guides” module on Benchling. sgRNAs were then cloned into the plenti U6-sgRNA/EF1a-Puro-2A-ZsGreen vector modified from the plenti U6-sgRNA/EF1a-mCherry vector (Addgene, plasmid no. 114199). Paired sgRNAs targeting BAX and BAK were designed and cloned into the abovementioned vector as described (Zou et al., “An APAF-1 Cytochrome c Multimeric Complex is a Functional Apoptosome that Activates Procaspase-9,” J. Biol. Chem. 274:11549-56 (1999), which is hereby incorporated by reference in its entirety). shRNAs were designed accordingly (Li et al., “Cytochrome c and dATP-Dependent Formation of Apaf-1/caspase-9 Complex Initiates an Apoptotic Protease Cascade,” Cell 91:479-89 (1997), which is hereby incorporated by reference in its entirety) and were cloned into the TRMPVIR vector as previously described (Pan et al., “Selective BCL-2 Inhibition by ABT-199 Causes on-Target Cell Death in Acute Myeloid Leukemia,” Cancer Discov. 4:362-75 (2014), which is hereby incorporated by reference in its entirety). All sgRNA and shRNA sequences used in this study are listed in Tables 2A-2C.

Competition-based survival assay and cell growth. For the competition-based survival assay, Cas9-expressing cells were transduced with the indicated sgRNAs. Cells were cultured for 8 days to allow complete CRISPR/Cas9 editing. Increasing amounts of Venetoclax were then added to the medium every other day at the following concentrations; for MOLM-13, 10 nM, 17 nM, 34 nM; for MV4:11, 17 nM, 34 nM, 68 nM for THP-1, 1000 nM, 2000 nM, 4000 nM. GFP⁺ percentages were assessed at 48 hrs upon each drug administration.

For cell growth assay, viable cells transduced with the indicated guides and puromycin selected were determined daily for 5-6 days by Trypan Blue exclusion.

Cell Cycle and Apoptosis. To assess cell cycle, we performed the 5-ethynyl-2′-deoxyuridine (EdU) incorporation assay using the Click-iT Plus EdU Alexa Fluor 647 kit as described in manufacturer's protocol (Life Technologies). Apoptosis analysis was determined using APC Annexin V (BD Bioscience) and 4′,6-Diamidino-2-Phenylindole, Dihydrochloride (DAPI) for DNA content. Both EdU and Annexin V stained cells were analyzed by flow cytometry and FlowJo software.

Biochemistry. Protein lysis was performed with RIPA buffer (Thermo Fisher Scientific) in presence of proteases inhibitor cocktail (PIC, Sigma) for 30 min at 4° C. After centrifugation at maximal speed for 20 min, supernatants were collected and protein concentration was determined using Bradford reagent (Bio-Rad).

Protein lysates (25-30 μg) were loaded onto 4%-12% Bis-Tris or 3%-8% Tris-acetate polyacrylamide gels (Thermo Fisher Scientific), transferred onto PVDF membranes (Millipore) and probed using the following antibodies: anti-CLPB polyclonal (1:2000, Proteintech, Cat #15743-1-AP), anti-TFAM (1:500, Santa Cruz, Cat #sc-376672), anti-OPA1 monoclonal (1:1000, BD Biosciences, Cat #612606), anti-SDHA monoclonal (1:2500, Abcam, Cat #ab14715), anti-HAX1 polyclonal (1:1000, Proteintech, Cat #11266-1-AP), anti-cytochrome c monoclonal (1:4000, Thermo Fisher Scientific, Cat #BDB556433), anti-caspase-3 monoclonal (1:1000, Proteintech, Cat #66470-2-Ig), anti-cleaved caspase-3 polyclonal (1:1000, Cell Signaling, Cat #9661S), anti-GRP75 monoclonal (1:1000, Santa Cruz, Cat #sc-133137), anti-TOM20 monoclonal (1:500, Santa Cruz, Cat #sc-17764), anti-p53 monoclonal (1:1000, Active Motif North America, Cat #39553), anti-BCL-2 polyclonal (1:200, R&D Systems, Cat #AF810-SP), anti-MCL1 polyclonal (1:1000, Proteintech, Cat #16225-1-AP), anti-BCL-XL monoclonal (1:500, R&D Systems, Cat #MAB894SP), anti-NOXA monoclonal (1:500, Abcam, Cat #ab13654), anti-GAPDH polyclonal (1:10000, Proteintech, Cat #10494-1-AP), anti-LAMIN A polyclonal (1:1000, Santa Cruz, Cat #sc-6214) and anti-ACTIN (1:10000, Millipore, Cat #MAB1501) antibodies. Isotype matched secondary antibodies were conjugated to horseradish peroxidase (GE Healthcare) and chemiluminescence was detected with ECL (Life Technologies) using films. Band densitometric analysis was performed using ImageJ (National Institutes of Health).

Mitochondrial Isolation and Cytochrome c Release Assay. Mitochondria from mouse liver and cell lines were isolated by standard differential centrifugation as previously described (DiNardo et al., “Safety and Preliminary Efficacy of Venetoclax with Decitabine or Azacitidine in Elderly Patients with Previously Untreated Acute Myeloid Leukaemia: a Non-Randomised, Open-Label, Phase 1b Study,” Lancet Oncol. 19:216-28 (2018), which is hereby incorporated by reference in its entirety). For cytochrome c release assay, p7/p15 recombinant BID was produced, purified and cleaved with caspase-8 as previously described (Bogenberger et al., “Ex Vivo Activity of BCL-2 Family Inhibitors ABT-199 and ABT-737 Combined with 5-azacytidine in Myeloid Malignancies,” Leuk Lymphoma 56:226-9 (2015), which is hereby incorporated by reference in its entirety). Mitochondria (1 mg/ml) were resuspended in experimental buffer (150 mM KCl, 10 mM Tris-Mops pH 7.4, 10 μM EGTA-Tris, 1 mM Pi and 5/2.5 mM Glutamate/Malate), treated with cleaved BID (30 pmol per mg of mitochondria), 30 min RT, and centrifuged at 12000×g for 10 min, 4° C. Equal amounts of pellet (mitochondria) and supernatant (released factors) were separated electrophoretically and immunoblotted against cytochrome c.

Immunoprecipitation. For immunoprecipitation in mouse liver mitochondria, isolated organelles were lysed in 150 mM NaCl, 25 mM Tris-Cl pH 7.4, 1 mM EDTA, 5% glycerol, 0.1% Triton X-100 in the presence of PIC. Lysates were pre-cleared with Dynabeads protein G (10 μl, Thermo Fisher Scientific) and incubated overnight with 6 μg of CLPB antibody (Proteintech). Next day, cell lysates containing antibodies were incubated with 50 μl Dynabeads, 90 min at RT, and washed three times with 1:5 diluted lysis buffer. The immunoprecipitated material was washed three times with 250 mM NaCl, 25 mM Tris-Cl pH 7.4, 1 mM EDTA, 0.05% Triton X-100. Bound proteins were eluted in 2×LDS sample buffer (NuPAGE) with 5% β-mercaptoethanol and 1% SDS, boiled and separated electrophoretically in a 4%-12% Bis-Tris gel (Thermo Fisher Scientific).

For immunoprecipitation in THP-1, 5×10⁸ cells were harvested, washed twice with 1×PBS and lysed in RIPA buffer (Thermo Fisher Scientific) supplemented with protease inhibitor cocktail (PIC, Sigma-Aldrich). Cell extracts were pre-cleared with Dynabeads protein G (10 μl, Thermo Fisher Scientific) and incubated with the antibody and Dynabeads as described above. Bound complexes were eluted twice in 2×LDS sample buffer (NuPAGE) with 5% 0-mercaptoethanol and 1% SDS by boiling, 10 mins at 70° C. Supernatant was taken for western blotting, silver staining using SilverQuest (Invitrogen) and mass spectrometry.

Preparation of Samples for Mass Spectrometry. For mass spectrometry, the samples were resuspended in NuPAGE LDS Sample Buffer (Novex). The proteins were reduced with 2 μl of 0.2M dithiothreitol (Sigma) for one hour at 57° C., pH 7.5. Next, the proteins were alkylated with 2μl of 0.5M iodoacetamide (Sigma) for 45 minutes at room temperature in the dark. The samples were loaded on a NuPAGE 4-12% Bis-Tris Gel 1.0 mm (Life Technologies) and run for 10 minutes at 200V. The gel was stained with GelCode Blue Stain Reagent (Thermo). The gel bands were excised, cut into 1 mm3 pieces and destained for 15 minutes in a 1:1 (v/v) solution of methanol and 100 mM ammonium bicarbonate. The buffer was exchanged and the samples were destained for another 15 minutes. This was repeated for another 3 cycles. The gel plugs were dehydrated by washing with acetonitrile, and further dried by placing them in a SpeedVac for 20 minutes. 300 ng of sequencing grade modified trypsin (Promega) was added directly to the dried gel pieces followed by enough 100 mM ammonium bicarbonate to cover the gel pieces. The gel plugs were allowed to shake at room temperature and digestion proceeded overnight. The digestion was halted by adding a slurry of R2 50 μm Poros beads (Applied Biosystems) in 5% formic acid and 0.2% trifluoroacetic acid (TFA) to each sample at a volume equal to that of the ammonium bicarbonate added for digestion. The samples were allowed to shake at 4° C. for 120 mins. The beads were loaded onto C18 ziptips (Millipore), equilibrated with 0.1% TFA, using a microcentrifuge for 30 s at 6,000 rpm. The beads were washed with 0.5% acetic acid. Peptides were eluted with 40% acetonitrile in 0.5% acetic acid followed by 80% acetonitrile in 0.5% acetic acid. The organic solvent was removed using a SpeedVac concentrator and the sample reconstituted in 0.5% acetic acid.

Mass Spectrometry Analysis and Data Processing. An aliquot of each sample was loaded onto an Acclaim PepMap trap column (2 cm×75 μm) in line with an EASY-Spray analytical column (50 cm×75 μm ID PepMap C18, 2 μm bead size) using the auto sampler of an EASY-nLC 1000 HPLC (ThermoFisher Scientific) with solvent A consisting of 2% acetonitrile in 0.5% acetic acid and solvent B consisting of 80% acetonitrile in 0.5% acetic acid. The peptides were gradient eluted into a ThermoFisher Scientific Orbitrap Fusion Lumos Mass Spectrometer using the following gradient: 5-35% in 60 min, 35-45% in 10 min, followed by 45-100% in 10 min, as previously described (Bogenberger et al., “BCL-2 Family Proteins as 5-Azacytidine-Sensitizing Targets and Determinants of Response in Myeloid Malignancies,” Leukemia 28:1657-65 (2014), which is hereby incorporated by reference in its entirety). High resolution full MS spectra were recorded with a resolution of 240,000, an AGC target of 1e6, with a maximum ion time of 50 ms, and a scan range from 400 to 1500 m/z. The MS/MS spectra were collected in the ion trap, with an AGC target of 2e4, maximum ion time of 18 ms, one microscan, 0.7 m/z isolation window, and Normalized Collision Energy (NCE) of 32.

All acquired MS2 spectra were searched against a UniProt human database using Sequest within Proteome Discoverer (Thermo Fisher Scientific). The search parameters were as follows: precursor mass tolerance±10 ppm, fragment mass tolerance 0.4 Da, digestion parameters trypsin allowing two missed cleavages, fixed modification of carbamidomethyl on cysteine, variable modification of oxidation on methionine, and variable modification of deamidation on glutamine and asparagine and a 1% peptide and protein FDR searched against a decoy database. The results were filtered to only include proteins identified by at least two unique peptides.

BH3 profiling assay. THP-1 transduced with sgRosa or sgCLPB were compared by BH3 profiling under basal conditions. For the assay, 3.5×10⁴ cells/well to 4×10⁴ cells/well were plated in a 384 well plate. BIM or BID peptide (final concentrations of 0.1-10 μM); PUMA, MS1 or BMF-7 peptides (final concentrations of 1-20 μM); Puma2A peptide (final concentration of 20 μM); alamethicin (final concentration of 25 μM); CCCP (final concentration of 10 μM) were added to JC1-MEB staining solution (150 mM mannitol, 10 mM HEPES-KOH, 50 mM KCl, 0.02 mM EGTA, 0.02 mM EDTA, 0.1% BSA, 5 mM succinate, pH 7.5) in a black 384-well plate. Single cell suspensions were prepared in JC-1-MEB buffer as previously described (Konopleva et al., “Efficacy and Biological Correlates of Response in a Phase II Study of Venetoclax Monotherapy in Patients with Acute Myelogenous Leukemia,” Cancer Discov. 6:1106-17 (2016), which is hereby incorporated by reference in its entirety). After adding the cells to the 384-well plate, fluorescence was measured at 590 nm emission 545 nM excitation using the TECAN M1000 microplate reader at 30° C. every 15 min for a total of 180 min.

Percentage of depolarization was calculated by normalization to the solvent-only control DMSO (0% depolarization) and the positive control CCCP (100% depolarization) as previously described (Ramsey et al., “A Novel MCL1 Inhibitor Combined with Venetoclax Rescues Venetoclax-Resistant Acute Myelogenous Leukemia,” Cancer Discov. 8:1566-81 (2018), which is hereby incorporated by reference in its entirety).

Immunofluorescence. HeLa or THP-1 cells were fixed on glass coverslips with freshly prepared, ice-cold 3.7% formaldehyde in PBS for 30 min, followed by permeabilization with 0.1% Triton X-100 in PBS for 10 min. Next, the coverslips were washed with PBS, incubated with blocking buffer (10% FBS/1% BSA in PBS) for 20 min and, then, with primary antibodies for 2 hrs at room temperature. Antibodies against CLPB (1:100, Proteintech) and TOM20 (1:100, Santa Cruz) diluted in the blocking buffer were used. The cells were then washed with PBS and incubated with the appropriate secondary antibodies labeled with FITC (1:500, for the antibody against CLPB) or Alexa Fluor 594 (1:500, for the antibody against TOM20) (Thermo Fisher) for 2 hrs at room temperature. The coverslips were mounted with one drop of Fluoromount-G (Southern Biotech). Fluorescence signals were analyzed under a ZEISS 710 confocal inverted microscope.

qRT-PCR Measurement of Gene Expression. RNA was extracted using the RNeasy Plus Mini Kit (QIAGEN) and reverse transcribed into cDNA using the High-Capacity cDNA Reverse Transcription Kit (ThermoFisher Scientific). Real-time PCR reactions were carried out using SYBR Green Master Mix (Roche) and run with a Lightcycler 480 II (Roche). The primer sequences used for cDNA quantification are shown in Tables 2A-2C. Relative expression levels across cell lines were calculated using the Delta-delta Ct method as per standard procedures.

RNA-Sequencing Library Preparation and Sequencing. For transcriptome analysis in CRISPR-mediated CLPB knockout (KO) AML cells and Venetoclax-resistant cell lines, cells were harvested at indicated time points and washed with 1×PBS. Total RNA was extracted using RNeasy Plus Mini Kit (Qiagen) and QIAshredder (Qiagen). Poly(A) mRNA was isolated with the magnetic isolation method (NEXTFLEX, Bioo Scientific) using ˜1 μg of total mRNA. To generate RNA-Sequencing libraries, the NEXTFLEX Rapid Directional RNA-Seq Library Prep Kit (Bioo Scientific) and NEXTFLEX RNA-Seq barcodes (Bioo Scientific) were used, carried out according to manufacturer's protocol. RNA-Seq barcoded libraries were then sequenced using Hi-Seq 4000 (single end, 50 cycles).

RNA-Sequencing Data Analysis, Ingenuity Pathway Analysis, Gene-set Enrichment Analysis and Gene Ontology Analyses. Based on RNA-Seq data of CLPB KO (sgCLPB #1 and #2) v.s. WT (sgRosa) at day 6 and day 8 post transduction, differential expression analysis was performed using edgeR package (3.24.1). 62 differentially expressed genes (FDR<0.05 and absolute log FC>1) across all 4 comparisons, i.e. sgCLPB #1 day 6 vs. sgRosa day 6, sgCLPB #1 day 8 vs. sgRosa day 8, sgCLPB #2 day 6 vs. sgRosa day 6 and sgCLPB #2 day 8 vs. sgRosa day 8, were visualized as a heatmap (pheatmap 1.0.10). To compare the transcriptome of the Venetoclax-resistant cell lines v.s. parental cell lines, RNA-Seq data were used for defining the differentially expressed genes. Differentially expressed genes were plotted using heatmap. Genes that involved in regulating mitochondrial biological processes were highlighted in the heatmap.

To define the “Genes encoding mitochondrial proteins”, the RNA-seq data for the list of ˜1500 human genes provided by the MitoMiner 4.0 database developed at the Mitochondrial Biology Unit at Cambridge, UK was filtered. MitoMiner is a database of mammalian mitochondrial localization evidence, which contains human genes that encode known mitochondrially localized proteins and proteins that are predicted to be mitochondrial using the evidence from the Integrated Mitochondrial Protein Index (IMPI).

Gene Ontology (GO) analyses and Kyoto Encyclopedia of Genes and Genomes (KEGG) enrichment analysis was performed for genes that are differentially expressed (FDR<0.05, indicated LFC) with R package clusterProfiler (3.10.0). Gene-set Enrichment Analysis (GSEA) was done using GSEA (Broad Institute) with a combined mitochondrial stress expression gene signature defined by Quirós et al (Tahir et al., “Potential Mechanisms of Resistance to Venetoclax and Strategies to Circumvent it,” BMC Cancer 17:399 (2017), which is hereby incorporated by reference in its entirety). Ingenuity Pathway Analysis (IPA) was performed for genes that are differentially expressed in CLPB KO (sgCLPB #1 and #2) v.s. WT (sgRosa) at day 6 post transduction (FDR<0.05, LFC>1 or <−1) using the IPA software developed by QIAGEN.

Metabolomics. Samples were subjected to an LCMS analysis to detect and quantify. Metabolite extraction was carried out on each sample with a previously described method (Pan et al., “Synthetic Lethality of Combined Bcl-2 Inhibition and p53 Activation in AML: Mechanisms and Superior Antileukemic Efficacy,” Cancer Cell 32:748-60 e6 (2017), which is hereby incorporated by reference in its entirety). The LC column was a ZIC-pHILIC (Millipore, 2.1×150 mm, 5 μm) coupled to a Dionex Ultimate 3000™ system and the column oven temperature was set to 25° C. for the gradient elution. A flow rate of 100 μL/min was used with the following buffers; A) 10 mM ammonium carbonate in water, pH 9.0, and B) neat acetonitrile. The gradient profile was as follows; 80-20% B (0-30 min), 20-80% B (30-31 min), 80-80% B (31-42 min). Injection volume was set to 1 μL for all analyses (42 min total run time per injection).

MS analyses were carried out by coupling the LC system to a Thermo Q Exactive HF™ mass spectrometer operating in heated electrospray ionization mode (HESI). Method duration was 30 min with a polarity switching data-dependent Top 5 method for both positive and negative modes. Spray voltage for both positive and negative modes was 3.5 kV and capillary temperature was set to 320° C. with a sheath gas rate of 35, aux gas of 10, and max spray current of 100 μA. The full MS scan for both polarities utilized 120,000 resolution with an AGC target of 3e6 and a maximum IT of 100 ms, and the scan range was from 67-1000 m/z. Tandem MS spectra for both positive and negative mode used a resolution of 15,000, AGC target of 1e5, maximum IT of 50 ms, isolation window of 0.4 m/z, isolation offset of 0.1 m/z, fixed first mass of 50 m/z, and 3-way multiplexed normalized collision energies (nCE) of 10, 35, 80. The minimum AGC target was 1e4 with an intensity threshold of 2e5. All data were acquired in profile mode. Top metabolites (differentially produced in either day 6 or day 8 CLPB knockout vs. wild-type and detected in at least ⅔ of samples) vs. differentially expressed genes were also visualized as heatmap. Spearman correlation of top metabolites with differentially expressed genes was calculated and the correlation coefficients were also plotted as heatmap. The “pathway analysis” module in MetaboAnalyst was used for analyzing the enriched metabolic pathways of the metabolomics data.

Mitochondrial Respiration. 250000 MOLM-13 cells per well were seeded in Cell Tak (ThermoFisher Scientific) coated XFe24 cell culture microplates, as indicated by the manufacturer. The experiment was performed in Cellular Assay Solution (RPMI 10-041-CV with 1 mM Sodium Pyruvate, pH 7.2). The assay consisted of oxygen consumption and extracellular acidification measurements during time starting with the basal conditions and followed by sequential injections of Oligomycin 1 μM. FCCP 2.2 μM and Rotenone/Antimycin 0.5 μM. Three measurements were performed after each compound injection. The analysis was performed simultaneously in the same plate for the control and CLPB knock-out cells.

ROS measurement. Mitochondrial reactive oxygen species (ROS) were measured by MitoSOX (Life Technologies). Cells transduced with sgRNAs targeting CLPB or control were loaded in 5 μM MitoSOX reagent in HBSS for 10 min at 37° C., light protected, and then washed three times with HBSS. MitoSOX fluorescence was measured by FACS at various time points with or without the addition of 10 μM Antimycin A (complex III inhibitor).

Animals. 12 weeks-old NOD SCID gamma (NSG) female mice were obtained from Jackson Laboratory. Mice were bred and maintained in individual ventilated cages and fed with autoclaved food and water at NYU School of Medicine Animal Facility. All animal experiments were performed in accordance with protocols approved by the New York University Institutional Animal Care and Use Committee (IACUC, ID: IA16-00008_TR1).

Statistical analysis. Kaplan-Meier survival curve p-values were performed using Log rank Mantel-COX test. For statistical comparison, we performed unpaired Student's t-test. Two-way ANOVA was used to the competition experiments. All statistical analyses were performed in Prism 7 software (GraphPad).

### Example 10—CLPB Inhibition can Re-Sensitize Venetoclax-Resistant Cells to Venetoclax-Based Therapies in Acute Myeloid Leukemia

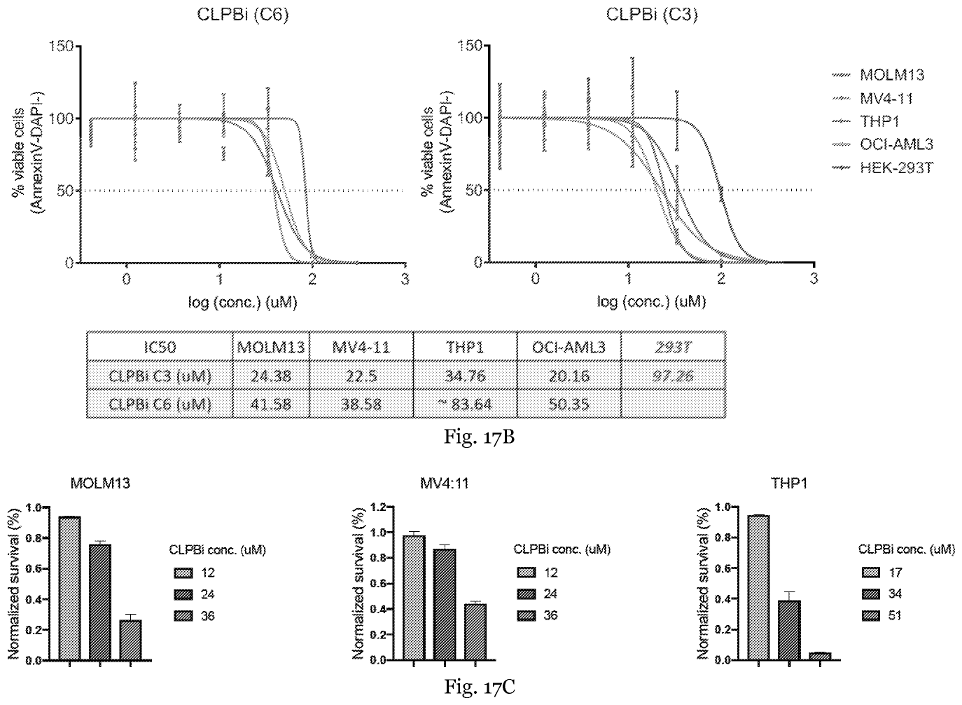

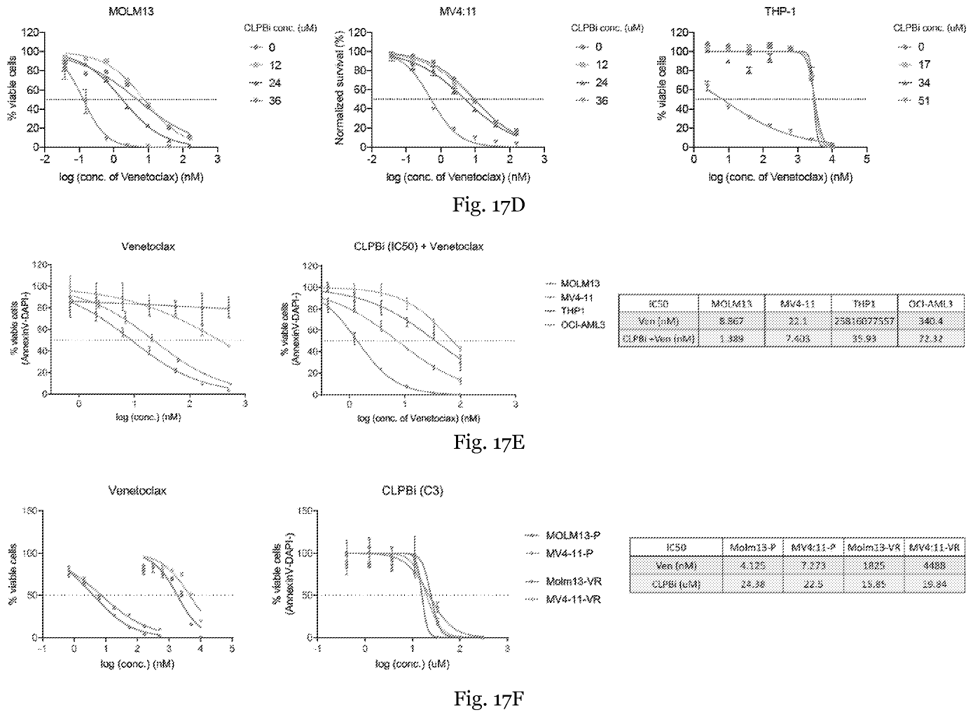

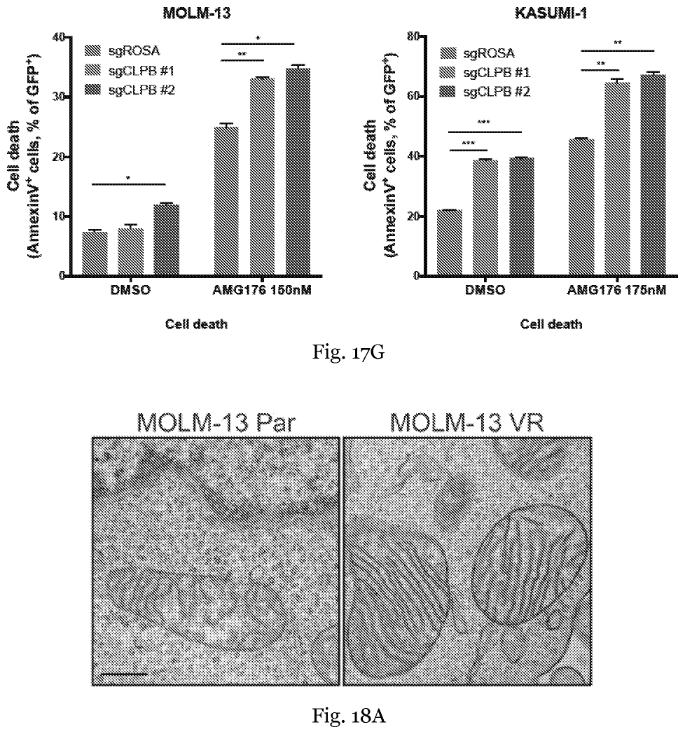

Viability experiments demonstrate that CLPB inhibition eliminates AML cells in vitro (FIGS. 17A, 17B). Cell death and survival experiments further showed that CLPB inhibition sensitizes human AML cells to Venetoclax treatment (FIGS. 17C-17E). Importantly, chemical targeting of CLPB using compound 3 is able to re-sensitize Venetoclax-resistant (VR) AML cells to Venetoclax treatment (FIG. 17F). Finally, CLPB depletion enhances the apoptosis induced by the MCL-1 inhibitor AMG176 in human AML cell lines, in a p53-independent way (FIG. 17G).

### Example 11—OPA1 Targeting Enhances the Apoptosis Induced by Venetoclax and Venetoclax+Azacitidine in AML

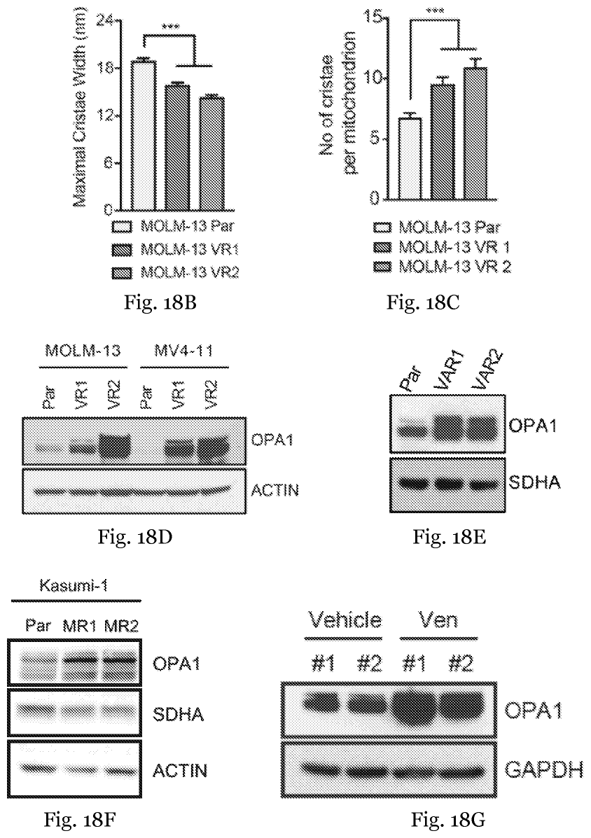

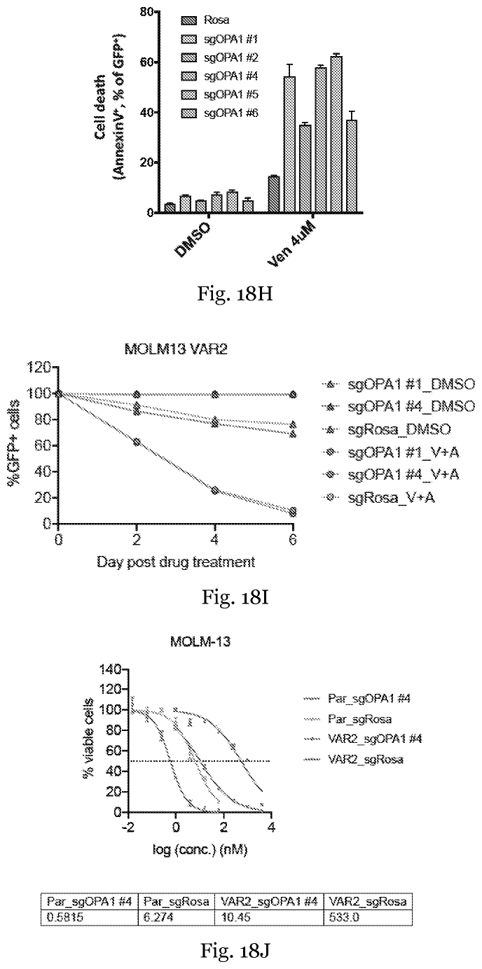

Further evidence links OPA1 upregulation and tighter mitochondrial cristae to resistance to BH3 mimetics-based therapies in human AML. Venetoclax resistance is strongly coupled to tighter mitochondrial cristae (FIGS. 18A-18C). In accordance with this, the protein levels of OPA1—the master regulator of cristae structure maintenance—significantly increase in: 1) Venetoclax-resistant AML cells (FIG. 18D), 2) Venetoclax+Azacitidine-resistant AML cells (FIG. 18E), 3) AMG176-resistant AML cells (FIG. 18F), and 4) MOLM-13 AML blasts from the bone marrow of wild-type mice treated with Venetoclax in late stages of AML (FIG. 18G).

Moreover, depletion of OPA1 enhances cell death induced by Venetoclax in AML cells (FIG. 18H). Furthermore, targeting OPA1 with sgRNAs sensitizes AML cells to Venetoclax and its combinations (FIG. 18I-18J). CLPB directly interacts with OPA1 to maintain the physiological mitochondrial morphology (Chen et al., “Targeting Mitochondrial Structure Sensitizes Acute Myeloid Leukemia to Venetoclax Treatment,” Cancer Discov. 9:890-909 (2019), which is hereby incorporated by reference in its entirety). Targeting of CLPB using small molecules will enhance OPA1-mediated mitochondrial cristae remodeling and cell death induced by BH3-mimetics, overcoming drug resistance.

### Example 12—Targeting Mitochondria Dynamics and Mitophagy Reverses BH3 Mimetics Drug Resistance in AML

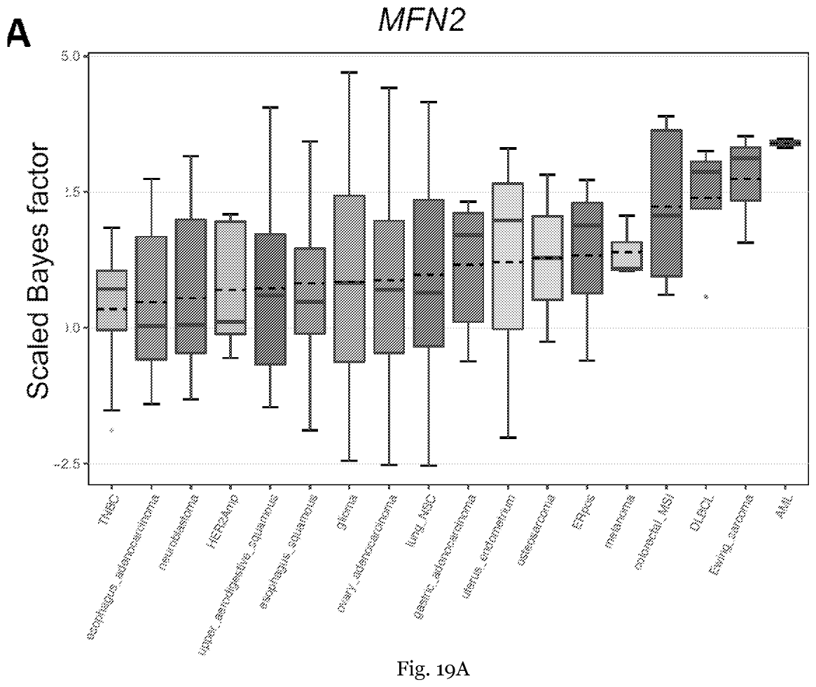

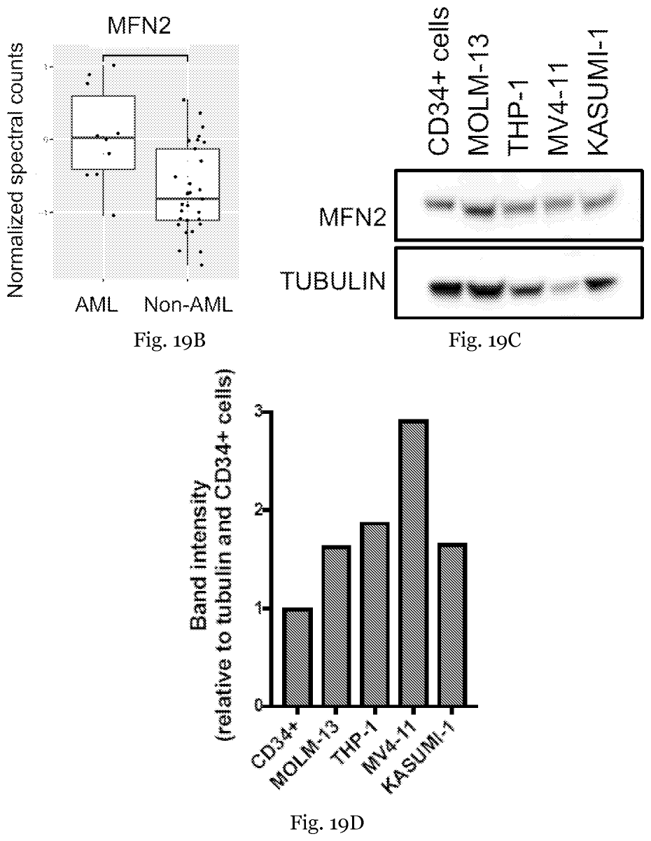

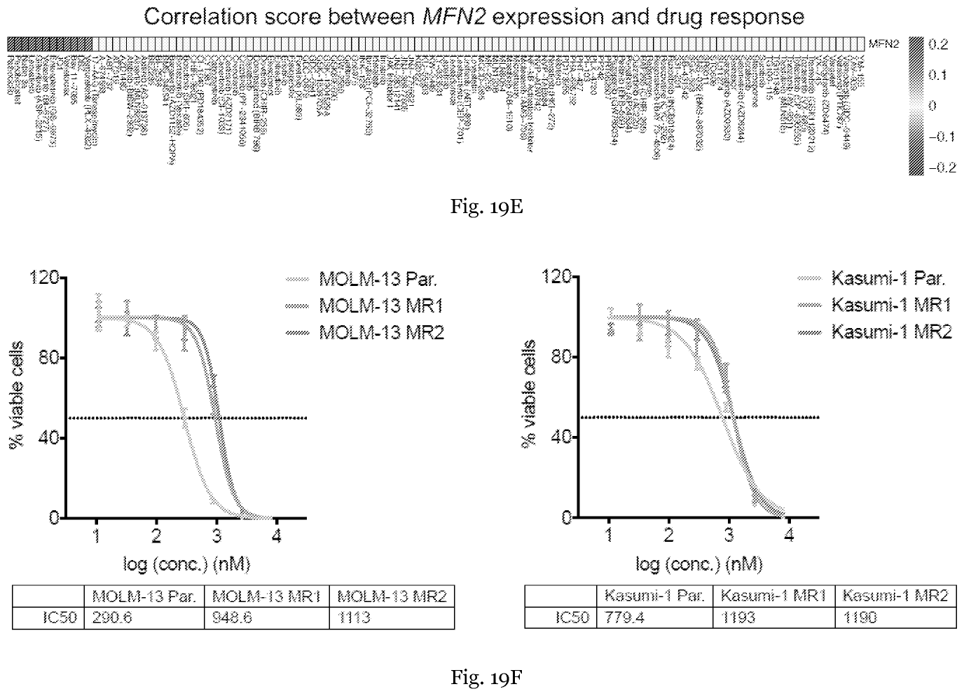

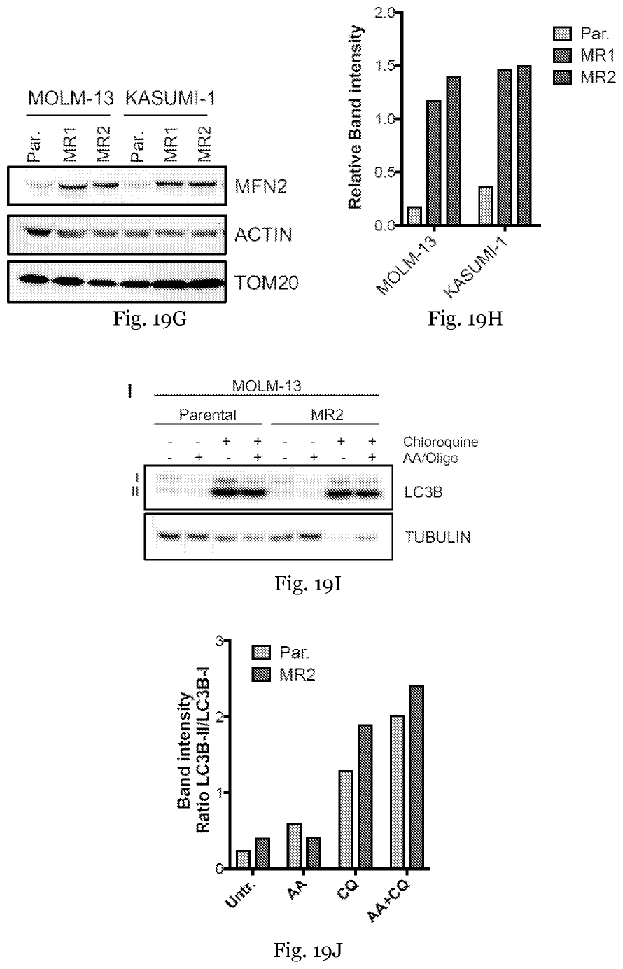

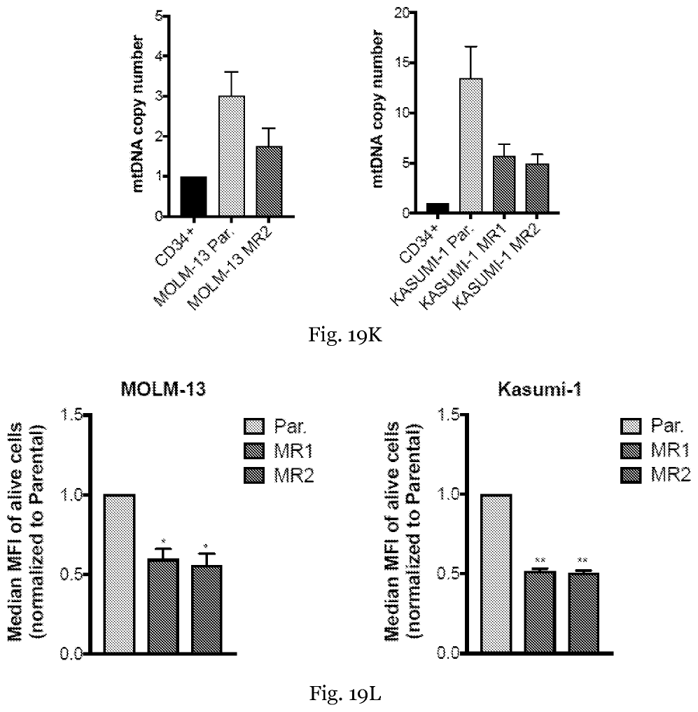

Autophagy inhibition can synergize with both Venetoclax (and its combinations), as well as with MCL1 inhibitors to enhance cell death and suppress AML growth. Autophagy is the natural, tightly-regulated mechanism of the cell that removes unnecessary or dysfunctional components. When this mechanism is used to clear defective mitochondria, the process is called mitophagy. Mitophagy is controlled by a number of mitochondrial proteins including mitofusin 2 (MFN2), one of the strongest hits in the CRISPRi screens that identify factors that cooperate with Venetoclax and MCL1 inhibitors. MFN2 is an outer mitochondrial GTPase, which plays critical roles in tethering mitochondria to the endoplasmic reticulum (de Brito et al., “Mitofusin 2 Tethers Endoplasmic Reticulum to Mitochondria,” Nature 456:605-10 (2008), which is hereby incorporated by reference in its entirety), while acting as a mark to mediate the degradation of damaged mitochondria via mitophagy (Chen et al., “PINK1-Phosphorylated Mitofusin 2 is a Parkin Receptor for Culling Damaged Mitochondria,” Science 340:471-75 (2013), which is hereby incorporated by reference in its entirety). MFN2 is an essential gene for acute myeloid leukemia compared to other tumors and its protein levels are elevated in human AML cell lines relative to other hematologic malignancies (Nusinow et al., “Quantitative Proteomics of the Cancer Cell Line Encyclopedia,” Cell 180: 387-402 e316 (2020), which is hereby incorporated by reference in its entirety) (FIGS. 19A, 19B). In addition, MFN2 protein levels are higher in human AML cells compared to healthy hematopoietic cells (CD34+), independently of the mutation status, further indicating an important role of MFN2 in AML (FIGS. 19C, 19D).

Furthermore, according to the Beat AML database of patients with AML (Tyner et al., “Functional Genomic Landscape of Acute Myeloid Leukaemia,” Nature 562:526-31 (2018), which is hereby incorporated by reference in its entirety), MFN2 mRNA levels positively correlate to resistance to a number of AML therapies, including venetoclax (FIG. 19E). Next, MCL-1 inhibitor (AMG176) resistant AML cell lines (MR) were generated (FIG. 19F). Importantly, a significant increase in MFN2 protein levels was observed in all the MR cell lines relative to the parental clones, suggesting that MFN2 participates in the acquisition of drug-resistance (FIGS. 19G, 19H).

Given the role of MFN2 in mitophagy, it was asked whether these resistant cells have higher autophagy flux compared to the parental AML cells. LC3 staining upon autophagy inhibition (chloroquine) or upon autophagy inhibition (chloroquine)+mitochondrial stress induction (antimycin A and oligomycin) indicated enhanced autophagy/mitophagy rates in the resistant cells compared to the parental cells (FIGS. 19I, 19J). In accordance with the above, resistant cells display lower mitochondrial mass, as indicated by the mtDNA copy number and mitoTracker staining (FIGS. 19K, 19L), further suggesting that resistance is accompanied by high mitophagy rates. Mitophagy has been reported to be a cytoprotective mechanism to eliminate damaged mitochondrial and block apoptosis (Marino et al., “Self-Consumption: the Interplay of Autophagy and Apoptosis,” Nat. Rev. Mol. Cell. Biol. 15:81-94 (2014), which is hereby incorporated by reference in its entirety). Therefore, increased mitophagy in the BH3-mimetics resistant AML cells might be a mechanism of evasion of apoptosis, by quickly removing the depolarized mitochondria.

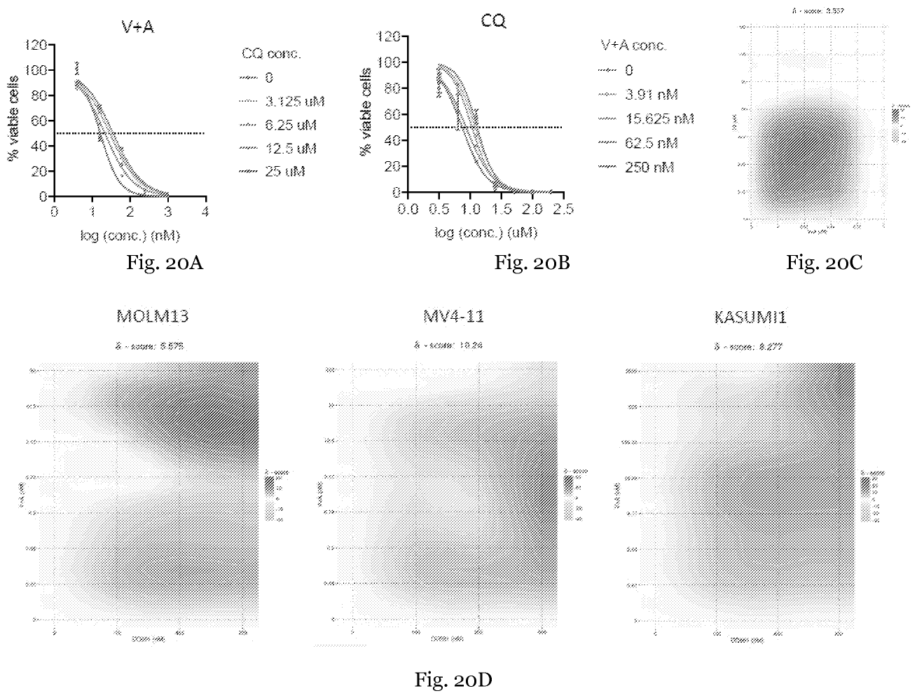

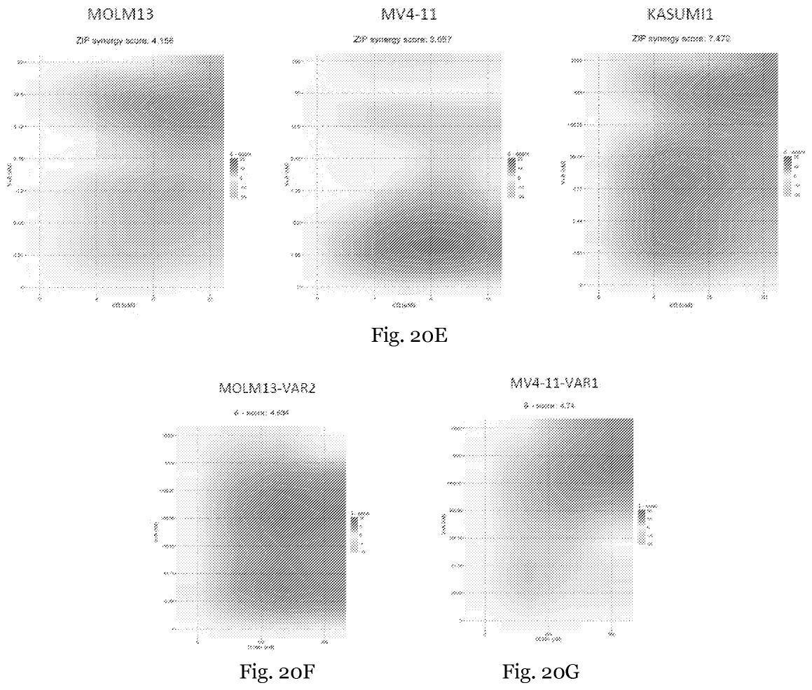

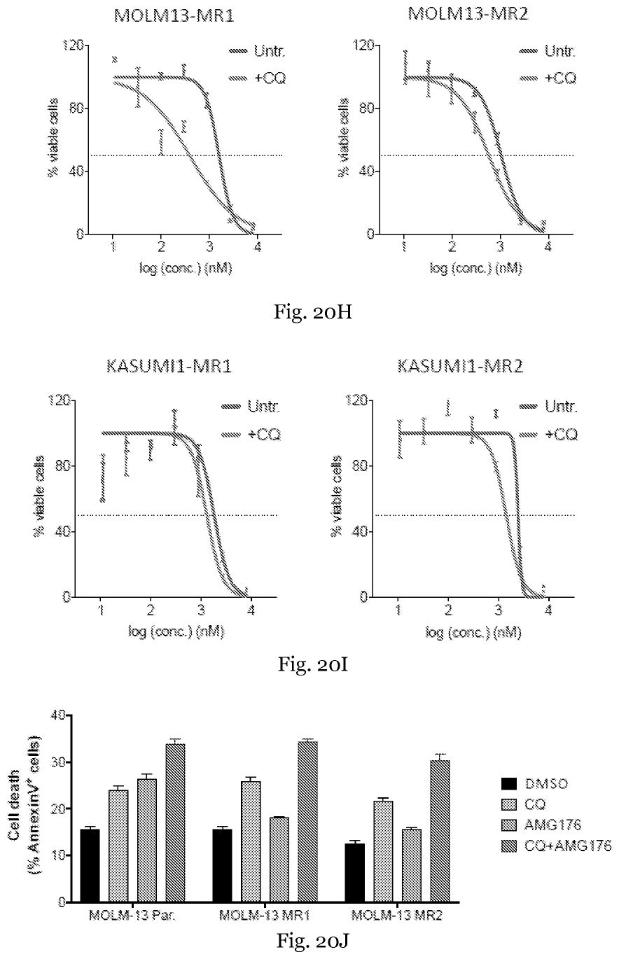

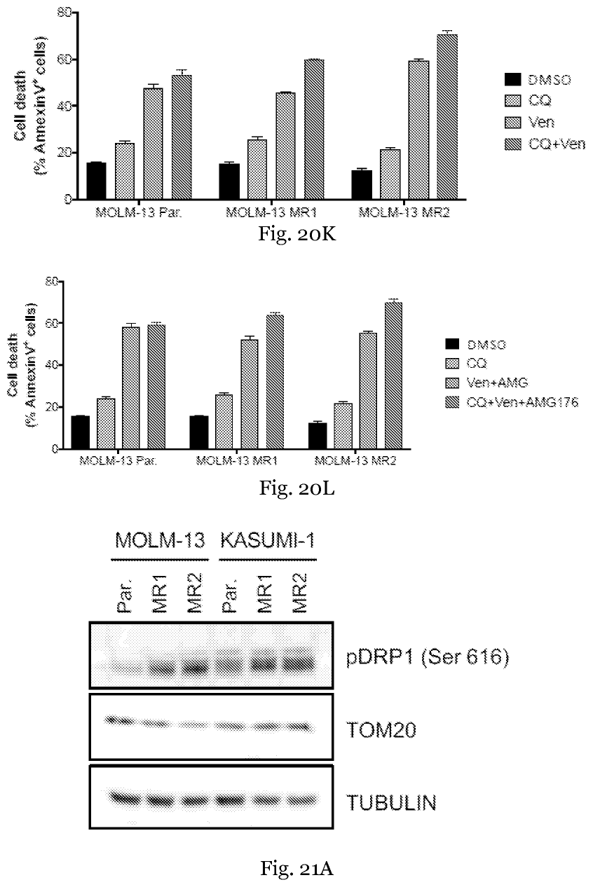

Next, it was asked whether autophagy inhibition might have a synthetic lethal activity together with BH3 mimetics in AML cells. The IC₅₀ of Venetoclax plus Azacitidine treatment (1:4 ratio) was significantly reduced upon addition of the autophagy inhibitor chloroquine in the murine MLL-AF9; Kras AML model (RN2), and vice versa (FIGS. 20A, 20B). Thus, the combination of Venetoclax plus Azacitidine and autophagy inhibitors can induce strong synergistic killing effect in AML (FIG. 20C). Furthermore, the synergistic effects of combining Venetoclax plus Azacitidine treatment and autophagy inhibitors (DC661 and Chloroquine) were also confirmed in multiple human AML cell lines (MOLM-13, MV4:11, THP-1 and KASUMI-1) in vitro with delta-scores from 3-10 (FIGS. 20D, 20E). The synergistic effects are maximized when each inhibitor is used at around their IC₅₀ values. More importantly, these synergistic effects can be confirmed in AML cells that are resistant to Venetoclax plus Azacitidine, suggesting that treatment of autophagy inhibitors can re-sensitize resistant AML cells to BH3 mimetics and their combinations (FIGS. 20F, 20G). Similarly, autophagy inhibition re-sensitizes AMG176-resistant AML cells to AMG176 or venetoclax, as demonstrated by survival curves (FIGS. 20H, 20I) and cell death experiments (annexin staining) (FIGS. 20J-20L).

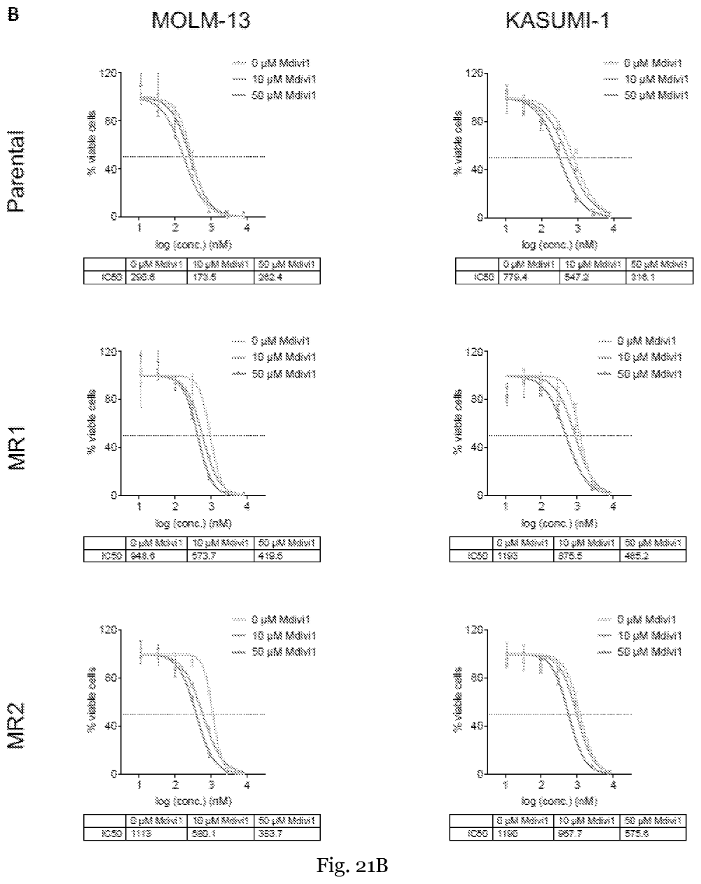

Finally, the phosphorylated form of DRP1 in Ser616 has been shown to be a marker of mitophagy (Saito et al., “An Alternative Mitophagy Pathway Mediated by Rab9 Protects the Heart Against Ischemia,” J. Clin. Invest. 129: 802-19 (2019), which is hereby incorporated by reference in its entirety). By western blotting, we observed elevated levels of phorsho-DRP1 (Ser616) in the AMG176-resistant cells compared to the parental clones (FIG. 21A), further indicating higher capacity of mitophagy. Importantly, Mdivi-1 (Mitochondrial division inhibitor-1), a specific DRP1 inhibitor, which blocks mitophagy, exhibited synergistic effects with AMG176 in AML cells (FIG. 21B). This mitophagy inhibitor might be another promising combination therapy with BH3 mimetics for patients with AML.

What is claimed is:

1. A method of increasing the sensitivity of cancer cells to cell death by an apoptosis-inducing drug, said method comprising:
administering to cancer cells an agent that modulates mitochondrial structure.

2. The method of claim 1, wherein the cancer cells are leukemic cells.

3. The method of claim 2, wherein the cancer cells are acute myeloid leukemia cells, chronic lymphocytic leukemia cells, T cell acute lymphocytic leukemia (T-ALL) cells, or early T cell progenitor leukemia (ETP-ALL) cells.

4. The method of claim 2, wherein the leukemic cells are leukemic stem cells.

5. The method of any one of claims 1-4, wherein the leukemic cells are p53 deficient leukemic cells.

6. The method of any one of claims 1-4, wherein the leukemic cells are p53 proficient leukemic cells.

7. The method of any one of claims 1-6, wherein the leukemic cells are mammalian cells.

8. The method of claim 7, wherein the mammalian cells are human cells.

9. The method of claim 1 or claim 2, wherein the cancer cells are resistant to drug-induced apoptosis, and said administering reverses said resistance.

10. The method of claim 1 or claim 2, wherein the apoptosis-inducing drug is a BH3 protein mimetic.

11. The method of claim 1 or claim 2, wherein the agent that modulates mitochondrial structure is an agent that inhibits the expression and/or activity of caseinolytic peptidase B protein homolog (CLPB).

12. The method of claim 11, wherein the agent that inhibits the expression and/or activity of CLPB is a compound of Formula I: 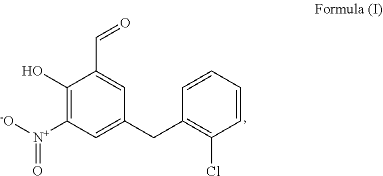
or a derivative thereof.

13. The method of claim 11, wherein the agent that inhibits the expression and/or activity of CLPB is a compound of Formula II: 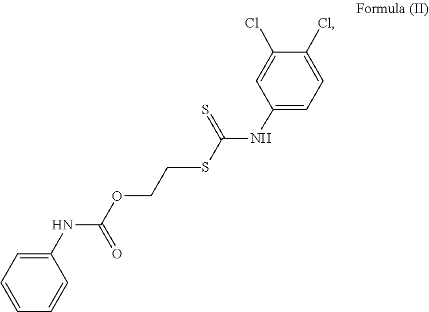 [SMILES: O=C(Nc1ccccc1)OCCCSC(=S)Nc1ccc(Cl)c(Cl)c1]
or a derivative thereof.

14. The method of claim 9, wherein the agent that inhibits the expression and/or activity of CLPB is guanidinium hydrochloride (CH₆ClN₃), or a derivative thereof.

15. A method of treating cancer in a subject, said method comprising:
selecting a subject having cancer; and
administering to said subject an agent that modulates mitochondrial structure.

16. The method of claim 15, wherein said cancer is leukemia.

17. The method of claim 16, wherein said selecting comprises:
selecting a patient having or at risk of having leukemia that is resistant to treatment with an apoptosis inducing drug.

18. The method of claim 17, wherein the leukemia is resistant to treatment with a BH3 protein mimetic.

19. The method of claim 18, wherein the BH3 protein mimetic inhibits BCL-2, BCL-XL, BCL-W, MCL-1, or combinations thereof.

20. The method of claim 18, wherein the wherein the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

21. The method of any one of claims 15-20, wherein said cancer is acute myeloid leukemia, chronic lymphocytic leukemia, T cell acute lymphocytic leukemia (T-ALL), or early T cell progenitor leukemia (ETP-ALL).

22. The method of any one of claims 15-21, wherein said agent is administered as part of a combination therapeutic, the combination therapeutic further comprising:
an apoptosis inducing drug.

23. The method of claim 22, wherein said agent and said apoptosis inducing drug are administered concurrently.

24. The method of claim 23, wherein said agent is administered prior to administering said apoptosis inducing drug.

25. The method of any one of claims 15-24, wherein the agent that modulates mitochondrial structure is an agent that inhibits expression and/or activity of CLPB, OPA1, HAX1, or combinations thereof.

26. The method of claim 24, wherein the agent that regulates mitochondrial structure is an agent that inhibits the expression and/or activity of caseinolytic peptidase B protein homolog (CLPB).

27. The method of claim 26, wherein the agent that inhibits the expression and/or activity of CLPB is a compound of Formula I: 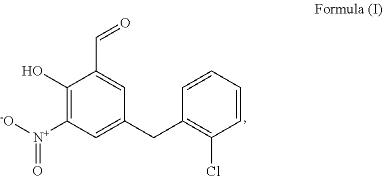
or a derivative thereof.

28. The method of claim 26, wherein the agent that inhibits the expression and/or activity of CLPB is a compound of Formula II: 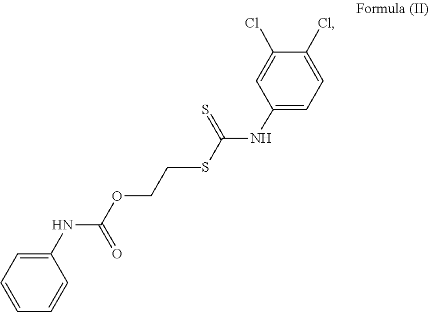 [SMILES: O=C(Nc1ccccc1)OCCCSC(=S)Nc1ccc(Cl)c(Cl)c1]
or a derivative thereof.

29. The method of claim 26, wherein the agent that inhibits the expression and/or activity of CLPB is guanidinium hydrochloride (CH₆ClN₃), or derivative thereof.

30. The method of claim 25, wherein the agent that regulates mitochondrial structure is an agent that inhibits the expression and/or activity of OPAL.

31. The method of claim 30, wherein the agent that inhibits the expression and/or activity of OPA1 is N-(1,5-dimethyl-3-oxo-2-phenyl-2,3-dihydro-1H-pyrazol-4-YL)-3-methyl-1-PH+ (MYLS22), or derivative thereof.

32. The method of any one of claims 22-30, wherein the apoptosis inducing drug is a BH3 protein mimetic.

33. The method of claim 32, wherein the BH3 protein mimetic inhibits BCL-2, BCL-XL, BCL-W, MCL-1, or a combination thereof.

34. The method of claim 32, wherein the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

35. The method of claim 34, wherein the BH3 protein mimetic is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax).

36. The method of any one of claims 22-35, wherein the combination therapeutic further comprises a chemotherapeutic drug.

37. The method of claim 36, wherein the chemotherapeutic drug is a hypomethylating agent.

38. The method of claim 37, wherein the hypomethylating agent is selected from 4-amino-1-[(2R,3R,4S,5R)-3,4-dihydroxy-5-(hydroxymethyl)oxolan-2-yl]-1,3,5-triazin-2-one (azacitidine), 4-amino-1-[(2R,4S,5R)-4-hydroxy-5-(hydroxymethyl)oxolan-2-yl]-1,3,5-triazin-2-one (decitabine), 4-amino-1-[(2R,3S,4S,5R)-3,4-dihydroxy-5-(hydroxymethyl)oxolan-2-yl]pyrimidin-2-one (cytarabine), (8S,10S)-10-{[(2R,4S,5S,6S)-4-amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione (doxorubicin), (8S,10S)-8-acetyl-10-{[(2R,4S,5S,6S)-4-amino-5-hydroxy-6-methyloxan-2-yl]oxy}-6,8,11-trihydroxy-1-methoxy-5,7,8,9,10,12-hexahydrotetracene-5,12-dione (daunorubicin), (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (epirubicin), (7S,9S)-9-acetyl-7-{[(2R,4S,5S,6S)-4-amino-5-hydroxy-6-methyloxan-2-yl]oxy}-6,9,11-trihydroxy-5,7,8,9,10,12-hexahydrotetracene-5,12-dione (idarubicin), anthracene-1,2-dione (anthracenedione), (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Adriamycin), 1,4-dihydroxy-5,8-bis({2-[(2-hydroxyethyl)amino]ethyl}amino)-9,10-dihydroanthracene-9,10-dione (mitoxantrone), and derivatives and/or combinations thereof.

39. The method of claim 38, wherein the hypomethylating agent is 4-amino-1-[(2R,3R,4S,5R)-3,4-dihydroxy-5-(hydroxymethyl)oxolan-2-yl]-1,3,5-triazin-2-one (azacitidine).

40. The method of any one of claims 36-39, wherein the combination therapeutic further comprises a CDK9 inhibitor and/or a MCL-1 inhibitor.

41. The method of claim 40, wherein the CDK9 inhibitor is selected from 2-(2-chlorophenyl)-5,7-dihydroxy-8-[(3S,4R)-3-hydroxy-1-methylpiperidin-4-yl]chromen-4-on (Alvocidib), (16E)-14-methyl-20-oxa-5,7,14,27-tetrazatetracyclo[19.3.1.12,6.18,12]heptacosa-1(25),2(27),3,5,8,10,12(26),16,21,23-decaene (Zotiraciclib), 4-[(2,6-dichlorobenzoyl)amino]-N-piperidin-4-yl-1H-pyrazole-5-carboxamide (AT-7519), (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide (AZD-4573), TP-1287, 2-[2-chloro-4-(trifluoromethyl)phenyl]-5,7-dihydroxy-8-[(2R,3S)-2-(hydroxymethyl)-1-methylpyrrolidin-3-yl]chromen-4-one (Voruciclib), 5-fluoro-4-(4-fluoro-2-methoxyphenyl)-N-[4-[(methylsulfonimidoyl)methyl]pyridin-2-yl]pyridin-2-amine (BAY-1251152), N-[5-[(5-tert-butyl-1,3-oxazol-2-yl)methylsulfanyl]-1,3-thiazol-2-yl]piperidine-4-carboxamide (SNS-032), (2R)-2-[[6-(benzylamino)-9-propan-2-ylpurin-2-yl]amino]butan-1-ol (Roscovitine), 2-[(2S)-1-[3-ethyl-7-[(1-oxidopyridin-1-ium-3-yl)methylamino]pyrazolo[1,5-a]pyrimidin-5-yl]piperidin-2-yl]ethanol (dinaciclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (Abemaciclib), 4-[(2,6-dichlorobenzoyl)amino]-N-piperidin-4-yl-1H-pyrazole-5-carboxamide (AT7519) and derivatives and/or combinations thereof.

42. The method of claim 40, wherein the MCL-1 inhibitor is selected from 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), N-[4-(2-tert-butylphenyl)sulfonylphenyl]-2,3,4-trihydroxy-5-[(2-propan-2-ylphenyl)methyl]benzamide (TW-37), (Z)-4-[(1S,2S,8R,17S,19R)-12-hydroxy-8,21,21-trimethyl-5-(3-methylbut-2-enyl)-8-(4-methylpent-3-enyl)-14,18-dioxo-3,7,20-trioxahexacyclo[15.4.1.0^{2,15}.0^{2,19}.0^{4,13}.0^{6,11}]docosa-4(13),5,9,11,15-pentaen-19-yl]-2-methylbut-2-enoic acid (Gambogic acid), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), [4,5-dichloro-1-[4,5-dichloro-2-(2-hydroxybenzoyl)-1H-pyrrol-3-yl]pyrrol-2-yl]-(2-hydroxyphenyl)methanone (maritoclax), 2-[4-[(4-bromophenyl)sulfonylamino]-1-hydroxynaphthalen-2-yl]sulfanylacetic acid (UMI-77), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), (2R)-2-[5-[3-chloro-2-methyl-4-[2-(4-methylpiperazin-1-yl)ethoxy]phenyl]-6-(4-fluorophenyl)thieno[2,3-d]pyrimidin-4-yl]oxy-3-[2-[[2-(2-methoxyphenyl)pyrimidin-4-yl]methoxy]phenyl]propanoic acid (MIK665/S64315), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD5991), and derivatives and/or combinations thereof.

43. The method of claim 22, wherein the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and a compound of Formula I:

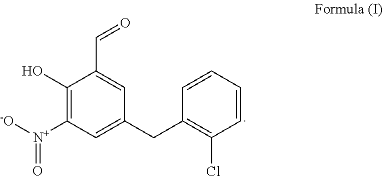

44. The method of claim 22, wherein the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), and a compound of Formula II:

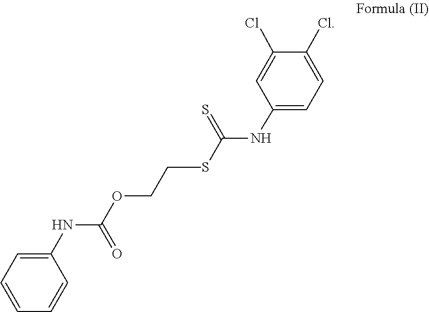 [SMILES: O=C(Nc1ccccc1)OCCCSC(=S)Nc1ccc(Cl)c(Cl)c1]

45. The method of claim 22, wherein the combination therapeutic comprises (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula I:

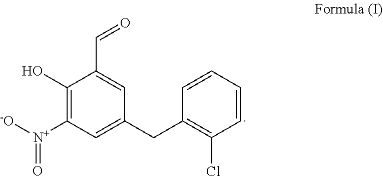

46. The method of claim 22, wherein the combination therapeutic comprises (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and a compound of Formula II:

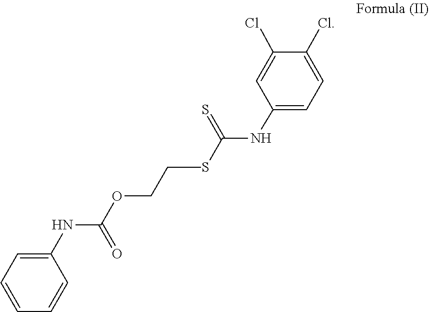 [SMILES: O=C(Nc1ccccc1)OCCCSC(=S)Nc1ccc(Cl)c(Cl)c1]

47. The method of any one of claims 43-46, wherein said combination therapeutic further comprises 4-amino-1-[(2R,3R,4S,5R)-3,4-dihydroxy-5-(hydroxymethyl)oxolan-2-yl]-1,3,5-triazin-2-one (azacitidine).

48. A combination therapeutic comprising:
an agent that modulates mitochondrial structure; and
an apoptosis inducing drug.

49. The combination therapeutic of claim 48, wherein the agent that modulates mitochondrial structure is an agent that inhibits the expression and/or activity of CLPB, OPA1, HAX1, or combinations thereof.

50. The combination therapeutic of claim 49, wherein the agent that regulates mitochondrial structure is an agent that inhibits the expression and/or activity of caseinolytic peptidase B protein homolog (CLPB).

51. The combination therapeutic of claim 50, wherein the agent that inhibits the expression and/or activity of CLPB is a compound of Formula I: 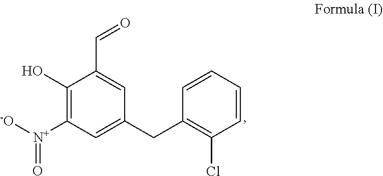
or a derivative thereof.

52. The combination therapeutic of claim 50, wherein the agent that inhibits the expression and/or activity of CLPB is a compound of Formula II: 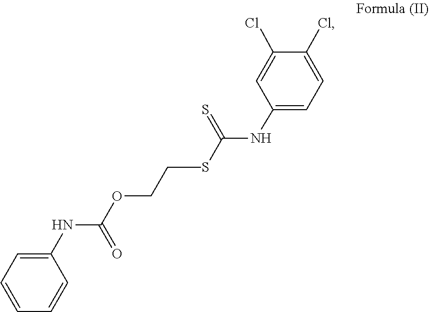 [SMILES: O=C(Nc1ccccc1)OCCCSC(=S)Nc1ccc(Cl)c(Cl)c1]
or a derivative thereof.

53. The combination therapeutic of claim 50, wherein the agent that inhibits the expression and/or activity of CLPB is guanidinium hydrochloride (CH₆ClN₃), or a derivative thereof.

54. The combination therapeutic of any one of claims 48-53, wherein the apoptosis inducing drug is a BH3-mimetic.

55. The combination therapeutic of claim 54, wherein the BH3-mimetic inhibits BCL-2, BCL-XL, BCL-W, MCL-1, or a combination thereof.

56. The combination therapeutic of claim 54, wherein the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

57. The combination therapeutic of claim 56, wherein the BH3-mimetic is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax).

58. The combination therapeutic of any one of claims 48-57, wherein the combination therapeutic further comprises a chemotherapeutic drug.

59. The combination therapeutic of claim 58, wherein the chemotherapeutic drug is a hypomethylating agent.

60. The combination therapeutic of claim 59, wherein the hypomethylating agent is selected from 4-amino-1-[(2R,3R,4S,5R)-3,4-dihydroxy-5-(hydroxymethyl)oxolan-2-yl]-1,3,5-triazin-2-one (azacitidine), 4-amino-1-[(2R,4S,5R)-4-hydroxy-5-(hydroxymethyl)oxolan-2-yl]-1,3,5-triazin-2-one (decitabine), 4-amino-1-[(2R,3S,4S,5R)-3,4-dihydroxy-5-(hydroxymethyl)oxolan-2-yl]pyrimidin-2-one (cytarabine), (8S,10S)-10-{[(2R,4S,5S,6S)-4-amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione (doxorubicin), (8S,10S)-8-acetyl-10-([(2R,4S,5S,6S)-4-amino-5-hydroxy-6-methyloxan-2-yl]oxy)-6,8,11-trihydroxy-1-methoxy-5,7,8,9,10,12-hexahydrotetracene-5,12-dione (daunorubicin), (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (epirubicin), (7S,9S)-9-acetyl-7-{[(2R,4S,5S,6S)-4-amino-5-hydroxy-6-methyloxan-2-yl]oxy}-6,9,11-trihydroxy-5,7,8,9,10,12-hexahydrotetracene-5,12-dione (idarubicin), anthracene-1,2-dione (anthracenedione), (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Adriamycin), 1,4-dihydroxy-5,8-bis((2-[(2-hydroxyethyl)amino]ethyl)amino)-9,10-dihydroanthracene-9,10-dione (mitoxantrone), and derivatives and/or combinations thereof.

61. The combination therapeutic of claim 60, wherein the hypomethylating agent is 4-amino-1-[(2R,3R,4S,5R)-3,4-dihydroxy-5-(hydroxymethyl)oxolan-2-yl]-1,3,5-triazin-2-one (azacitidine).

62. The combination therapeutic of any one of claims 58-61, wherein the combination therapeutic further comprises a CDK9 inhibitor and/or a MCL-1 inhibitor.

63. The combination therapeutic of claim 62, wherein the CDK9 inhibitor is selected from 2-(2-chlorophenyl)-5,7-dihydroxy-8-[(3S,4R)-3-hydroxy-1-methylpiperidin-4-yl]chromen-4-on (Alvocidib), (16E)-14-methyl-20-oxa-5,7,14,27-tetrazatetracyclo[19.3.1.12,6.18,12]heptacosa-1(25),2(27),3,5,8,10,12(26),16,21,23-decaene (Zotiraciclib), 4-[(2,6-dichlorobenzoyl)amino]-N-piperidin-4-yl-1H-pyrazole-5-carboxamide (AT-7519), (1S,3R)-3-acetamido-N-[5-chloro-4-(5,5-dimethyl-4,6-dihydropyrrolo[1,2-b]pyrazol-3-yl)pyridin-2-yl]cyclohexane-1-carboxamide (AZD-4573), TP-1287, 2-[2-chloro-4-(trifluoromethyl)phenyl]-5,7-dihydroxy-8-[(2R,3S)-2-(hydroxymethyl)-1-methylpyrrolidin-3-yl]chromen-4-one (Voruciclib), 5-fluoro-4-(4-fluoro-2-methoxyphenyl)-N-[4-[(methylsulfonimidoyl)methyl]pyridin-2-yl]pyridin-2-amine (BAY-1251152), N-[5-[(5-tert-butyl-1,3-oxazol-2-yl)methylsulfanyl]-1,3-thiazol-2-yl]piperidine-4-carboxamide (SNS-032), (2R)-2-[[6-(benzylamino)-9-propan-2-ylpurin-2-yl]amino]butan-1-ol (Roscovitine), 2-[(2S)-1-[3-ethyl-7-[(1-oxidopyridin-1-ium-3-yl)methylamino]pyrazolo[1,5-a]pyrimidin-5-yl]piperidin-2-yl]ethanol (dinaciclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (Abemaciclib), 4-[(2,6-dichlorobenzoyl)amino]-N-piperidin-4-yl-1H-pyrazole-5-carboxamide (AT7519) and derivatives and/or combinations thereof.

64. The combination therapeutic of claim 62, wherein the MCL-1 inhibitor is selected from 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), N-[4-(2-tert-butylphenyl)sulfonylphenyl]-2,3,4-trihydroxy-5-[(2-propan-2-ylphenyl)methyl]benzamide (TW-37), (Z)-4-[(1S,2S,8R,17S,19R)-12-hydroxy-8,21,21-trimethyl-5-(3-methylbut-2-enyl)-8-(4-methylpent-3-enyl)-14,18-dioxo-3,7,20-trioxahexacyclo[15.4.1.0^{2,15}.0^{2,19}.0^{4,13}.0^{6,11}]docosa-4(13),5,9,11,15-pentaen-19-yl]-2-methylbut-2-enoic acid (Gambogic acid), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), [4,5-dichloro-1-[4,5-dichloro-2-(2-hydroxybenzoyl)-1H-pyrrol-3-yl]pyrrol-2-yl]-(2-hydroxyphenyl)methanone (maritoclax), 2-[4-[(4-bromophenyl)sulfonylamino]-1-hydroxynaphthalen-2-yl]sulfanylacetic acid (UMI-77), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), (2R)-2-[5-[3-chloro-2-methyl-4-[2-(4-methylpiperazin-1-yl)ethoxy]phenyl]-6-(4-fluorophenyl)thieno[2,3-d]pyrimidin-4-yl]oxy-3-[2-[[2-(2-methoxyphenyl)pyrimidin-4-yl]methoxy]phenyl]propanoic acid (MIK665/S64315), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD5991), and derivatives and/or combinations thereof.

65. The combination therapeutic of any one of claims 48-64, wherein the agent and the apoptosis inducing drug are formulated together in a single pharmaceutical composition.

66. The combination therapeutic of any one of claims 48-64, wherein the agent and the apoptosis inducing drug are formulated as separate pharmaceutical compositions.

67. A method of treating cancer in a subject, said method comprising:
selecting a subject having cancer; and
administering to said subject a combination therapeutic comprising a BH3 protein mimetic and an agent that blocks autophagy.

68. The method of claim 67, wherein the cancer is leukemia.

69. The method of claim 68, wherein said selecting comprises:
selecting a patient having or at risk of having leukemia that is resistant to treatment with a BH3 protein mimetic.

70. The method of claim 69, wherein the leukemia is resistant to treatment with a BH3 protein mimetic selected from 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), or derivatives thereof.

71. The method of any one of claims 67-70, wherein said leukemia is acute myeloid leukemia, T cell acute lymphocytic leukemia (T-ALL), early T cell progenitor leukemia (ETP-ALL), or chronic lymphocytic leukemia.

72. The method of claim 67, wherein the agent that blocks autophagy is an endosomal acidification inhibitor and/or deacidifier, a PI3K inhibitor, a MAPK inhibitor, a mitophagy inhibitor, a VPS34 inhibitor, ATG4B inhibitor, USP10 and/or USP13 inhibitor, or combination thereof.

73. The method of claim 72, wherein the endosomal acidification inhibitor and/or deacidifier is N′-(7-chloroquinolin-4-yl)-N,N-diethylpentane-1,4-diamine (chloroquine), 2-[4-[(7-chloroquinolin-4-yl)amino]pentyl-ethylamino]ethanol (hydroxychloroquine); N-(7-chloroquinolin-4-yl)-N′-[6-[(7-chloroquinolin-4-yl)amino]hexyl]-N′-methylhexane-1,6-diamine (DC661), (3Z,5E,7R,8S,9S,11E,13E,15S,16R)-16-[(2S,3R,4S)-4-[(2R,4R,5S,6R)-2,4-dihydroxy-5-methyl-6-propan-2-yloxan-2-yl]-3-hydroxypentan-2-yl]-8-hydroxy-3,15-dimethoxy-5,7,9,11-tetramethyl-1-oxacyclohexadeca-3,5,11,13-tetraen-2-one (Bafilomycin A1), N-(7-chloroquinolin-4-yl)-N′-[2-[(7-chloroquinolin-4-yl)amino]ethyl]-N′-methylethane-1,2-diamine;trihydrochloride (Lys05), or derivatives thereof.

74. The method of claim 72, wherein the PI3K inhibitor is 3-methyl-7H-purin-6-imine (3-Methyladenin), 2-morpholin-4-yl-8-phenylchromen-4-one (LY294002), or derivatives thereof.

75. The method of claim 72, wherein the MAPK inhibitor is 4-[5-(4-fluorophenyl)-4-(pyridin-4-yl)-1H-imidazol-2-yl]phenol (SB202190), 4-[4-(4-fluorophenyl)-2-(4-methanesulfinylphenyl)-1H-imidazol-5-yl]pyridine (SB203580), or derivatives thereof.

76. The method of claim 72, wherein the mitophagy inhibitor is 3-(2,4-dichloro-5-methoxyphenyl)-2-sulfanylidene-1H-quinazolin-4-one (Mdivi-1), or derivative thereof.

77. The method of claim 72, wherein the ATG4B inhibitor is N-pyridin-2-ylpyridine-2-carbothioamide (NSC 185058), 3-[[4-[(E)-2-(7-chloroquinolin-4-yl)ethenyl]phenyl]-(2-phenylethylsulfanyl)methyl]sulfanylpropanoic acid (LV-320), or derivatives thereof.

78. The method of claim 72, wherein the USP10 and/or USP13 inhibitor is 6-fluoro-N-[(4-fluorophenyl)methyl]quinazolin-4-amine (Spautin-1), or derivative thereof.

79. The method of claim 67, wherein the BH3 protein mimetic inhibits BCL-2, BCL-XL, BCL-W, MCL-1, or a combination thereof.

80. The method of claim 67, wherein the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-136-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

81. The method of claim 80, wherein the BH3 protein mimetic is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax).

82. The method of claim 67, wherein said administering comprises administering the BH3 protein mimetic and the agent that blocks autophagy of the combination therapeutic concurrently.

83. The method of claim 67, wherein said administering comprises administering the agent that blocks autophagy prior to the BH3 protein mimetic.

84. The method of any one of claims 67-83, wherein the combination therapeutic further comprises a chemotherapeutic drug.

85. The method of claim 84, wherein the chemotherapeutic drug is a hypomethylating agent.

86. The method of claim 85, wherein the hypomethylating agent is selected from 4-amino-1-[(2R,3R,4S,5R)-3,4-dihydroxy-5-(hydroxymethyl)oxolan-2-yl]-1,3,5-triazin-2-one (azacitidine), 4-amino-1-[(2R,4S,5R)-4-hydroxy-5-(hydroxymethyl)oxolan-2-yl]-1,3,5-triazin-2-one (decitabine), 4-amino-1-[(2R,3S,4S,5R)-3,4-dihydroxy-5-(hydroxymethyl)oxolan-2-yl]pyrimidin-2-one (cytarabine), (8S,10S)-10-{[(2R,4S,5S,6S)-4-amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione (doxorubicin), (8S,10S)-8-acetyl-10-{[(2R,4S,5S,6S)-4-amino-5-hydroxy-6-methyloxan-2-yl]oxy}-6,8,11-trihydroxy-1-methoxy-5,7,8,9,10,12-hexahydrotetracene-5,12-dione (daunorubicin), (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (epirubicin), (7S,9S)-9-acetyl-7-([(2R,4S,5S,6S)-4-amino-5-hydroxy-6-methyloxan-2-yl]oxy)-6,9,11-trihydroxy-5,7,8,9,10,12-hexahydrotetracene-5,12-dione (idarubicin), anthracene-1,2-dione (anthracenedione), (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Adriamycin), 1,4-dihydroxy-5,8-bis({2-[(2-hydroxyethyl)amino]ethyl}amino)-9,10-dihydroanthracene-9,10-dione (mitoxantrone), and derivatives and/or combinations thereof.

87. The method of claim 86, wherein the hypomethylating agent is 4-amino-1-[(2R,3R,4S,5R)-3,4-dihydroxy-5-(hydroxymethyl)oxolan-2-yl]-1,3,5-triazin-2-one (azacitidine).

88. The method of claim 67, wherein the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), and N′-(7-chloroquinolin-4-yl)-N,N-diethylpentane-1,4-diamine (chloroquine).

89. The method of claim 84, wherein the combination therapeutic comprises 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), 4-amino-1-[(2R,3R,4S,5R)-3,4-dihydroxy-5-(hydroxymethyl)oxolan-2-yl]-1,3,5-triazin-2-one (azacitidine), and N′-(7-chloroquinolin-4-yl)-N,N-diethylpentane-1,4-diamine (chloroquine).

90. The method of claim 67, wherein the combination therapeutic comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax).

91. The method of claim 67, wherein the combination therapeutic comprises N-(7-chloroquinolin-4-yl)-N′-[6-[(7-chloroquinolin-4-yl)amino]hexyl]-N′-methylhexane-1,6-diamine (DC661) and 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax) and 4-amino-1-[(2R,3R,4S,5R)-3,4-dihydroxy-5-(hydroxymethyl)oxolan-2-yl]-1,3,5-triazin-2-one (azacitidine).

92. The method of claim 67, wherein the combination therapeutic comprises N′-(7-chloroquinolin-4-yl)-N,N-diethylpentane-1,4-diamine (chloroquine) and +(3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176).

93. The method of claim 84, wherein the combination therapeutic comprises N′-(7-chloroquinolin-4-yl)-N,N-diethylpentane-1,4-diamine (chloroquine), (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 4-amino-1-[(2R,3R,4S,5R)-3,4-dihydroxy-5-(hydroxymethyl)oxolan-2-yl]-1,3,5-triazin-2-one (azacitidine).

94. The method of claim 67, wherein the combination therapeutic comprises (3′R,4S,6′R,7S,8′E,11′S,12′R)-7-chloro-7′-methoxy-11′,12′-dimethyl-13′,13′-dioxospiro[2,3-dihydro-1H-naphthalene-4,22′-20-oxa-13λ6-thia-1,14-diazatetracyclo[14.7.2.03,6.019,24]pentacosa-8,16(25),17,19(24)-tetraene]-15′-one (AMG-176), and 3-(2,4-dichloro-5-methoxyphenyl)-2-sulfanylidene-1H-quinazolin-4-one (Mdivi-1).

95. A combination therapeutic comprising:
an agent that blocks autophagy; and
a BH3 protein mimetic.

96. The combination therapeutic of claim 95, wherein the agent that blocks autophagy is an endosomal acidification inhibitor and/or deacidifier, a PI3K inhibitor, a MAPK inhibitor, a mitophagy inhibitor, a VPS34 inhibitor, ATG4B inhibitor, USP10 and/or USP13 inhibitor, or combination thereof.

97. The combination therapeutic of claim 96, wherein the endosomal acidification inhibitor and/or deacidifier is N′-(7-chloroquinolin-4-yl)-N,N-diethylpentane-1,4-diamine (chloroquine), 2-[4-[(7-chloroquinolin-4-yl)amino]pentyl-ethylamino]ethanol (hydroxychloroquine); N-(7-chloroquinolin-4-yl)-N′-[6-[(7-chloroquinolin-4-yl)amino]hexyl]-N′-methylhexane-1,6-diamine (DC661), (3Z,5E,7R,8S,9S,11E,13E,15S,16R)-16-[(2S,3R,4S)-4-[(2R,4R,5S,6R)-2,4-dihydroxy-5-methyl-6-propan-2-yloxan-2-yl]-3-hydroxypentan-2-yl]-8-hydroxy-3,15-dimethoxy-5,7,9,11-tetramethyl-1-oxacyclohexadeca-3,5,11,13-tetraen-2-one (Bafilomycin A1), N-(7-chloroquinolin-4-yl)-N′-[2-[(7-chloroquinolin-4-yl)amino]ethyl]-N′-methylethane-1,2-diamine;trihydrochloride (Lys05), or derivatives thereof.

98. The combination therapeutic of claim 96, wherein the P3K inhibitor is 3-methyl-7H-purin-6-imine (3-Methyladenin), 2-morpholin-4-yl-8-phenylchromen-4-one (LY294002), or derivatives thereof.

99. The combination therapeutic of claim 96, wherein the MAPK inhibitor is 4-[5-(4-fluorophenyl)-4-(pyridin-4-yl)-1H-imidazol-2-yl]phenol (SB202190), 4-[4-(4-fluorophenyl)-2-(4-methanesulfinylphenyl)-1H-imidazol-5-yl]pyridine (SB203580), or derivatives thereof.

100. The combination therapeutic of claim 96, wherein the mitophagy inhibitor is 3-(2,4-dichloro-5-methoxyphenyl)-2-sulfanylidene-1H-quinazolin-4-one (Mdivi-1), or derivative thereof.

101. The combination therapeutic of claim 96, wherein the ATG4B inhibitor is N-pyridin-2-ylpyridine-2-carbothioamide (NSC 185058), 3-[[4-[(E)-2-(7-chloroquinolin-4-yl)ethenyl]phenyl]-(2-phenylethylsulfanyl)methyl]sulfanylpropanoic acid (LV-320), or derivatives thereof.

102. The combination therapeutic of claim 96, wherein the USP10 and/or USP13 inhibitor is 6-fluoro-N-[(4-fluorophenyl)methyl]quinazolin-4-amine (Spautin-1), or derivative thereof.

103. The combination therapeutic of claim 95, wherein the BH3 protein mimetic inhibits BCL-2, BCL-XL, BCL-W, MCL-1, or a combination thereof.

104. The combination therapeutic of claim 84, wherein the BH3 protein mimetic is 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide (navitoclax), 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax), N-(4-hydroxyphenyl)-3-[6-[(3S)-3-(morpholin-4-ylmethyl)-3,4-dihydro-1H-isoquinoline-2-carbonyl]-1,3-benzodioxol-5-yl]-N-phenyl-5,6,7,8-tetrahydroindolizine-1-carboxamide;hydrochloride (S55746, BLC201), (2R)-2-[5-[3-chloro-2-methyl-4-[2-(4-methylpiperazin-1-yl)ethoxy]phenyl]-6-(5-fluorofuran-2-yl)thieno[2,3-d]pyrimidin-4-yl]oxy-3-[2-[[2-(2,2,2-trifluoroethyl)pyrazol-3-yl]methoxy]phenyl]propanoic acid (S63845), (3′R,4S,6′R,7′S,8′E,11′S,12′R)-7-chloro-7′-methoxy-11′,12′-dimethyl-13′,13′-dioxospiro[2,3-dihydro-1H-naphthalene-4,22′-20-oxa-136-thia-1,14-diazatetracyclo[14.7.2.03,6.019,24]pentacosa-8,16(25),17,19(24)-tetraene]-15′-one (AMG-176), 17-chloro-5,13,14,22-tetramethyl-28-oxa-2,9-dithia-5,6,12,13,22-pentazaheptacyclo[27.7.1.1^{4,7}.0^{11,15}.0^{16,21}.0^{20,24}.0^{30,35}]octatriaconta-1(36),4(38),6,11,14,16,18,20,23,29(37),30,32,34-tridecaene-23-carboxylic acid (AZD-5991), 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide (ABT-737), 2-[(5E)-5-[(4-bromophenyl)methylidene]-4-oxo-2-sulfanylidene-1,3-thiazolidin-3-yl]-3-methylbutanoic acid (BH3I-1), 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde (AT101), 3-[1-(1-adamantylmethyl)-5-methylpyrazol-4-yl]-6-[8-(1,3-benzothiazol-2-ylcarbamoyl)-3,4-dihydro-1H-isoquinolin-2-yl]pyridine-2-carboxylic acid (A-1331852), 2-[8-(1,3-benzothiazol-2-ylcarbamoyl)-3,4-dihydro-1H-isoquinolin-2-yl]-5-[3-[4-[3-(dimethylamino)prop-1-ynyl]-2-fluorophenoxy]propyl]-1,3-thiazole-4-carboxylic acid (A-1155463), N-[4-(2-tert-butylphenyl)sulfonylphenyl]-2,3,4-trihydroxy-5-[(2-propan-2-ylphenyl)methyl]benzamide (TW-37), 7-[5-[[4-[4-(dimethylsulfamoyl)piperazin-1-yl]phenoxy]methyl]-1,3-dimethylpyrazol-4-yl]-1-(2-morpholin-4-ylethyl)-3-(3-naphthalen-1-yloxypropyl)indole-2-carboxylic acid (A-1210477), 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide (Sabutoclax), or derivatives thereof.

105. The combination therapeutic of claim 104, wherein the BH3 protein mimetic is 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide (venetoclax).

106. The combination therapeutic of any one of claims 95-105, wherein the combination therapeutic further comprises a chemotherapeutic drug.

107. The combination therapeutic of claim 106, wherein the chemotherapeutic drug is a hypomethylating agent.

108. The combination therapeutic of claim 106, wherein the hypomethylating agent is selected from 4-amino-1-[(2R,3R,4S,5R)-3,4-dihydroxy-5-(hydroxymethyl)oxolan-2-yl]-1,3,5-triazin-2-one (azacitidine), 4-amino-1-[(2R,4S,5R)-4-hydroxy-5-(hydroxymethyl)oxolan-2-yl]-1,3,5-triazin-2-one (decitabine), 4-amino-1-[(2R,3S,4S,5R)-3,4-dihydroxy-5-(hydroxymethyl)oxolan-2-yl]pyrimidin-2-one (cytarabine), (8S,10S)-10-{[(2R,4S,5S,6S)-4-amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione (doxorubicin), (8S,10S)-8-acetyl-10-([(2R,4S,5S,6S)-4-amino-5-hydroxy-6-methyloxan-2-yl]oxy)-6,8,11-trihydroxy-1-methoxy-5,7,8,9,10,12-hexahydrotetracene-5,12-dione (daunorubicin), (7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (epirubicin), (7S,9S)-9-acetyl-7-{[(2R,4S,5S,6S)-4-amino-5-hydroxy-6-methyloxan-2-yl]oxy}-6,9,11-trihydroxy-5,7,8,9,10,12-hexahydrotetracene-5,12-dione (idarubicin), anthracene-1,2-dione (anthracenedione), (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Adriamycin), 1,4-dihydroxy-5,8-bis((2-[(2-hydroxyethyl)amino]ethyl)amino)-9,10-dihydroanthracene-9,10-dione (mitoxantrone), and derivatives and/or combinations thereof.

109. The combination therapeutic of claim 108, wherein the hypomethylating agent is 4-amino-1-[(2R,3R,4S,5R)-3,4-dihydroxy-5-(hydroxymethyl)oxolan-2-yl]-1,3,5-triazin-2-one (azacitidine).
